(19) United States
(12) Patent Application Publication
(10) Pub. No.: US 2023/0355599 A1
(43) Pub. Date: Nov. 9, 2023

(54) BIOMARKERS FOR CBL, AND COMPOSITIONS AND METHODS FOR THEIR USE

(71) Applicant: NURIX THERAPEUTICS, INC., San Francisco, CA (US)

(72) Inventors: Janine POWERS, San Francisco, CA (US); Sarah Anne WHELAN, San Francisco, CA (US)

(21) Appl. No.: 18/302,603

(22) Filed: Jul. 24, 2023

Related U.S. Application Data
(60) Provisional application No. 63/332,523, filed on Apr. 19, 2022.
(60) Provisional application No. 63/339,866, filed on May 9, 2022.

(51) Int. Cl. A61K 31/454; G01N 33/574; A61P 35/00

(52) U.S. Cl. CPC A61K 31/454; G01N 33/5748; G01N 33/57492; A61P 35/00; G01N 2800/52; G01N 2333/912; G01N 2440/14

(57) ABSTRACT

Provided herein are biomarkers, compositions and methods for the measurement of CBL in samples, for instance in vivo samples.

### CROSS-REFERENCE TO RELATED APPLICATIONS

This application claims the benefit of U.S. provisional application No. 63/332,523, filed Apr. 19, 2022, and U.S. provisional application No. 63/339,866, filed May 9, 2022, the contents of which are hereby incorporated by reference in their entireties.

### FIELD

Provided herein are biomarkers for CBL inhibition, methods of their use for measuring CBL activity in vivo, and compositions and methods for their use in therapy.

### BACKGROUND

Casitas B-lineage lymphoma proto-oncogene (CBL) has been characterized as a regulator of the adaptive immune response (Bachmaier et al., 2000, Nature 403(6766):211-6; Chiang et al., 2000, Nature 403(6766):216-20). CBL functions as an intracellular checkpoint that regulates T cell activation, NK cell activity, and immune response through degradation of specific proteins. Both T cell receptor (TCR) signaling and B cell receptor (BCR) signaling are modulated by CBL mediated ubiquitination. Recent studies have identified CBL inhibition as a promising target for therapy. Inhibition of CBL with a small molecule drug activates T cells, a goal in the treatment of cancer, where T cells and the entire immune system can be mobilized to destroy a tumor. However, detection and measurement of CBL inhibition in vivo can be challenging. Practical in vivo markers for CBL are needed for the study and use of these promising CBL therapeutics.

### SUMMARY

The methods and compositions provided herein are based, in part, on the discovery of proximal biomarkers of CBL inhibition that show statistically significant correlation with activity in vivo. As demonstrated in the examples provided herein, statistically significant increases of phosphorylated interleukin-2-inducible kinase (ITK), phosphorylated hematopoietic lineage cell-specific protein (HS1), phosphorylated phospholipase C2 (PLCγ2), phosphorylated phospholipase C1 (PLCγ1), phosphorylated spleen tyrosine kinase (SYK), and phosphorylated Zeta-chain-associated protein kinase 70 (ZAP70) correlated with CBL inhibition in vitro and in vivo. ITK is a T-cell-specific kinase inducible by interleukin 2 (Siliciano et al., 1992, Proc Natl Acad Sci USA 89(23):11194-8). HS1 binds the actin cytoskeleton and regulates TCR activation through the immune synapse (Yamanashi et al., 1993, Proc. Natl. Acad. Sci. USA 90(8):3631-5; Scaife et al., 2003, J. Cell Sci. 116(3): 463-473; Gomez et al., 2006, Immunity 24:741-752; Carrizosa et al., 2009, J. Immunol. 183(11):7352-61). SYK is a tyrosine kinase involved in TCR signaling (Chan et al., 1994, J. Immunol. 152(10):4758-4766). PLCγ2, PLCγ1, and ZAP70 are expressed in T cells and activated upon TCR stimulation. (Rhee et al., 2001, Annual Review of Biochemistry. 70:281-312; Fu et al., 2010, J. Experimental Med. 207(2):309-318; Wang et al., 2010, Cold Spring Harbor Persp. Biol. 2(5):a002279).

In one aspect, provided herein are methods of measuring proximal biomarkers of CBL inhibition in a sample. In certain embodiments, the methods comprise the steps of measuring the amount of a proximal biomarker of CBL inhibition in the sample. In certain embodiments, the CBL is Cbl-b. In certain embodiments, the CBL is c-Cbl. In certain embodiments, the CBL is Cbl-b and c-Cbl. In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70.

In one aspect, provided herein are methods for detecting the amount of biomarkers of CBL inhibition in a cell, tissue, or organism. In certain embodiments, the methods comprise the steps of measuring the amount of a proximal biomarker of CBL inhibition in a first sample of the cell, tissue, or organism; measuring the amount of a proximal biomarker of CBL inhibition in a second sample of the cell, tissue, or organism; and identifying a change in the amount of CBL in the cell, tissue, or organism based on the change in the amount of the biomarker detected in the sample. In certain embodiments, the CBL is Cbl-b. In certain embodiments, the CBL is c-Cbl. In certain embodiments, the CBL is Cbl-b and c-Cbl. In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70.

In one aspect, provided herein are methods of treating a patient in need thereof with a CBL inhibitor. In certain embodiments, the methods comprise administering to a patient a first dose of a CBL inhibitor; measuring the amount of a CBL biomarker in a first sample of the patient; and identifying the amount of CBL in the patient based on the amount of the CBL biomarker detected. In certain embodiments, the CBL is Cbl-b. In certain embodiments, the CBL is c-Cbl. In certain embodiments, the CBL is Cbl-b and c-Cbl. In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70.

In one aspect, provided herein are methods of treating a patient in need thereof with a CBL inhibitor. In certain embodiments, the methods comprise administering to a patient a first dose of a CBL inhibitor; measuring the amount of a CBL biomarker in a first sample of the cell, tissue, or organism; measuring the amount of a CBL biomarker in a second sample of the cell, tissue, or organism; and identifying a change in the amount of CBL in the cell, tissue, or organism based on the change in the amount of the CBL biomarker detected in the sample. In certain embodiments, the CBL is Cbl-b. In certain embodiments, the CBL is c-Cbl. In certain embodiments, the CBL is Cbl-b and c-Cbl. In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70.

In another aspect, provided herein are compositions and kits for carrying out the methods. The compositions and methods are useful for treating or preventing any disease or disorder modulated by CBL. In certain embodiments, the compositions and methods are useful for treating a proliferative disorder. In certain embodiments, the CBL inhibitor is a small molecule. In certain embodiments, CBL inhibitor is a small molecule administered in combination with a cell therapy. Useful CBL inhibitors and therapies are described herein.

### BRIEF DESCRIPTION OF THE FIGURES

FIG. 1A provides single dose Compound 23 in cynomolgus monkeys induces phosphorylated PLCγ2 upon stimulation of circulating CD8⁺ T cells.

FIG. 1B provides single dose Compound 23 in cynomolgus monkeys induces phosphorylated HS1 upon stimulation of circulating CD8⁺ T cells.

FIG. 1C provides single dose Compound 23 in cynomolgus monkeys induces phosphorylated HS1 upon stimulation of circulating CD8⁺ T cells.

FIG. 2A provides multiple doses of Compound 23 in cynomolgus monkeys induces phosphorylated PLCγ2 upon stimulation of circulating CD8⁺ T cells.

FIG. 2B provides multiple doses of Compound 23 in cynomolgus monkeys induces phosphorylated HS1 upon stimulation of circulating CD8⁺ T cells.

FIG. 2C provides multiple doses of Compound 23 in cynomolgus monkeys induces phosphorylated ZAP70 upon stimulation of circulating CD8⁺ T cells.

FIG. 3A provides Compound 23 induces phosphorylated HS1 upon stimulation in vitro in human CD8⁺ T cells from whole blood.

FIG. 3B provides Compound 23 induces phosphorylated PLCγ2 upon stimulation in vitro in human CD8⁺ T cells from whole blood.

FIG. 3C provides Compound 23 induces phosphorylated ZAP70 upon stimulation in vitro in human CD8⁺ T cells from whole blood.

FIG. 4A provides Compound 23 induces phosphorylated HS1 upon stimulation in vitro in cynomolgus macaque CD8⁺ T cells from whole blood.

FIG. 4B provides Compound 23 induces phosphorylated PLCγ2 upon stimulation in vitro in cynomolgus macaque CD8⁺ T cells from whole blood.

FIG. 4C provides Compound 23 induces phosphorylated ZAP70 upon stimulation in vitro in cynomolgus macaque CD8⁺ T cells from whole blood.

FIG. 5A provides an illustration of methods for identifying proximal biomarkers of Compound 23 described herein.

FIG. 5B provides results of the methods, identifying markers pHS1, PLCγ2, and pZAP70.

FIG. 5C provides results of the methods, identifying markers pITK, pHS1, PLCγ1, PLCγ2, pSYK, and pZAP70.

FIG. 6A provides dose dependent activation of Compound 23 proximal biomarkers pHS1, PLCγ2, and pZAP70 with a 5-minute stimulation.

FIG. 6B provides dose dependent activation of Compound 23 proximal biomarkers pHS1, PLCγ2, and pZAP70 with a 60-minute stimulation.

FIG. 7 provides Compound 23 eliciting a concentration-dependent increase in phosphorylated HS1 in human whole blood.

FIG. 8A provides dose-dependent phosphorylation of HS1 on administration of Compound 23 to human patients.

FIG. 8B provides maximal percent HS1 phosphorylation on administration of Compound 23 to human patients.

FIG. 8C provides area under the curve percent phosphorylated HS1 on administration of Compound 23 to human patients.

FIG. 9A provides maximal percent PLCγ2 phosphorylation on administration of Compound 23 to human patients.

FIG. 9B provides area under the curve percent phosphorylated PLCγ2 on administration of Compound 23 to human patients.

FIG. 10A provides maximal percent ZAP70 phosphorylation on administration of Compound 23 to human patients.

FIG. 10B provides area under the curve percent phosphorylated ZAP70 on administration of Compound 23 to human patients.

### DETAILED DESCRIPTION

Provided herein are biomarkers, compositions, and methods useful for treating proliferative disorders in a subject.

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

It is understood that aspects and embodiments described herein as “comprising” include “consisting of” and “consisting essentially of” embodiments.

As used herein and in the appended claims, the singular forms “a,” “an,” and “the” include plural referents unless otherwise indicated or clear from context. For example, “an” excipient includes one or more excipients.

Reference to “about” a value, encompasses from 90% to 110% of that value. For instance, about 50 billion cells refers to 45 to 55 billion cells, and includes 50 billion cells. For instance, a temperature of “about 100 degrees” refers to a temperature of about 90 degrees to about 110 degrees.

When numerical ranges of compounds are given, all compounds within those numerical limits designated “a” and “b” are included, unless expressly excluded. For example, reference to compounds 9-13 refers to compounds 9, 10, 11, 12, and 13.

The term “CBL” refers to any Casitas B-lineage lymphoma gene encoding one of the CBL family of proteins. CBL family proteins include c-Cbl and Cbl-b.

The term “Cbl-b” as used herein refers to a Cbl-b protein. The term also includes naturally occurring variants of Cbl-b, including splice variants or allelic variants. The term also includes non-naturally occurring variants of Cbl-b, such as a recombinant Cbl-b protein or truncated variants thereof, which generally preserve the binding ability of naturally occurring Cbl-b or naturally occurring variants of Cbl-b (e.g., the ability to bind to an E2 enzyme). Sequences include NM_170662, NM_001321786, NM_001321788, NM_001321789, NM_001321790, and NM_001033238 (mRNA); and NP_001308715, NP_001308717, NP_001308718, NP_001308719, and NP_001308720 (protein).

The term “c-Cbl” as used herein refers to a c-Cbl protein. The term also includes naturally occurring variants of c-Cbl, including splice variants or allelic variants. The term also includes non-naturally occurring variants of c-Cbl, such as a recombinant c-Cbl protein or truncated variants thereof, which generally preserve the binding ability of naturally occurring c-Cbl or naturally occurring variants of c-Cbl (e.g., the ability to bind to an E2 enzyme). Sequences include NM_005188 human and NM_007619 mouse(mRNA); and NP_005179 human and NP_013645 mouse (protein).

The term “ITK” as used herein refers to a interleukin-2-inducible T-cell kinase. In humans, ITK is encoded by the ITK gene. The term “pITK” refers to phosphorylated ITK. The term also includes naturally occurring variants of ITK, including splice variants or allelic variants. Sequences include NM_005546 (mRNA); and NP_005537 (protein). In certain embodiments, pITK is phosphorylated on residue Y180.

The term “HS1” as used herein refers to a hematopoietic lineage cell-specific protein. In humans, HS1 is encoded by the HCLS1 gene. The term “pHS1” refers to phosphorylated HS1. The term also includes naturally occurring variants of HS1, including splice variants or allelic variants. Sequences include NM_005335 and NM_001292041 (mRNA); and NP_005326 and NP_0001278970 (protein). In certain embodiments, pHS1 is phosphorylated on residue Y397.

The term “PLCγ2” as used herein refers to a phosphatidylinositol-specific phospholipase C, gamma 2 protein. In humans, pPLCγ2 is encoded by the PLCG2 gene. The term “pPLCγ2” refers to phosphorylated PLCγ2. The term also includes naturally occurring variants of PLCγ2, including splice variants or allelic variants. Sequences include NM_002661 (mRNA); and NP_002652 (protein). In certain embodiments, PLCγ2 is phosphorylated on residue Y759.

The term “PLCγ1” as used herein refers to a phosphatidylinositol-specific phospholipase C, gamma 1 protein. In humans, pPLCγ1 is encoded by the PLCG1 gene. The term “pPLCγ1” refers to phosphorylated PLCγ1. The term also includes naturally occurring variants of PLCγ1, including splice variants or allelic variants. Sequences include NM_002660 and NM_182811 (mRNA); and NP_002651 and NP_877963 (protein). In certain embodiments, PLCγ1 is phosphorylated on residue Y783.

The term “SYK” as used herein refers to a tyrosine-protein kinase, or spleen tyrosine kinase, protein. In humans, SYK is encoded by the SYK gene. The term “pSYK” refers to phosphorylated SYK. The term also includes naturally occurring variants of SYK, including splice variants or allelic variants. Sequences include NM_001135052, NM_001174167, NM_001174168, and NM_003177 (mRNA); and NP_001128524, NP_001167638, NP_001167639, and, NP_003168 (protein). In certain embodiments, pSYK is phosphorylated on residue Y525, Y526, and/or Y348.

The term “ZAP70” as used herein refers to a zeta-chain-associated protein kinase 70 protein. In humans, ZAP70 is encoded by the ZAP70 gene. The term “pZAP70” refers to phosphorylated ZAP70. The term also includes naturally occurring variants of ZAP70, including splice variants or allelic variants. Sequences include NM_001079, NM_207519, and NM_001378594 (mRNA); and NP_001070, NP_997402, NP_001365523 (protein). In certain embodiments, pZAP70 is phosphorylated on residue Y319.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions may be sterile. Such formulations or compositions may be sterile, with the exception of the inclusion of an oncolytic virus.

“Excipients” as used herein include pharmaceutically acceptable excipients, carriers, vehicles, or stabilizers that are nontoxic to the cell or mammal being exposed thereto at the dosages and concentrations employed. Often the physiologically acceptable excipient is an aqueous pH buffered solution.

Reference to a compound as described in a pharmaceutical composition, or to a compound as described in a claim to a pharmaceutical composition, refers to the compound described by the formula recited in the pharmaceutical composition, without the other elements of the pharmaceutical composition, that is, without carriers, excipients, etc.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “inhibits growth” (e.g. referring to cells, such as tumor cells) is intended to include any measurable decrease in cell growth (e.g., tumor cell growth) when contacted with a compound or combination described herein, as compared to the growth of the same cells not in contact with the same compound or combination. In certain embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In certain embodiments, the subject has a disease that can be treated or diagnosed with a CBL inhibitor provided herein. In certain embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

An “effective amount” of an agent disclosed herein is an amount sufficient to carry out a specifically stated purpose. An “effective amount” may be determined empirically and in a routine manner, in relation to the stated purpose. An “effective amount” or an “amount sufficient” of an agent is that amount adequate to produce a desired biological effect, such as a beneficial result, including a beneficial clinical result. In certain embodiments, the term “effective amount” refers to an amount of an agent effective to “treat” a disease or disorder in an individual (e.g., a mammal such as a human).

“Proliferation” is used herein to refer to the proliferation of a cell. Increased proliferation encompasses the production of a greater number of cells relative to a baseline value. Decreased proliferation encompasses the production of a reduced number of cells relative to a baseline value. In certain embodiments, the cell is an immune cell such as a T-cell and increased proliferation is desired. In certain embodiments, the cell is a cancer cell and reduced proliferation is desired.

“Alkyl” as used herein refers to a saturated linear (i.e., unbranched) or branched univalent hydrocarbon chain or combination thereof. Particular alkyl groups are those having a designated number of carbon atoms, for example, an alkyl group having 1 to 20 carbon atoms (a “C₁-C₂₀ alkyl”), having 1 to 10 carbon atoms (a “C₁-C₁₀” alkyl), having 1 to 8 carbon atoms (a “C₁-C₈ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combination thereof, having at least one site of olefinic unsaturation (i.e., having at least one moiety of the formula C═C). Particular alkenyl groups are those having a designated number of carbon atoms, for example, an alkenyl group having 2 to 20 carbon atoms (a “C₂-C₂₀ alkenyl”), having 2 to 10 carbon atoms (a “C₂-C₁₀” alkenyl), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). The alkenyl group may be in “cis” or “trans” configurations or, alternatively, in “E” or “Z” configurations. Examples of alkenyl groups include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e., unbranched) or branched univalent hydrocarbon chain or combination thereof, having at least one site of acetylenic unsaturation (i.e., having at least one moiety of the formula C≡C). Particular alkynyl groups are those having a designated number of carbon atoms, for example, an alkynyl group having 2 to 20 carbon atoms (a “C₂-C₂₀ alkynyl”), having 2 to 10 carbon atoms (a “C₂-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs, and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”), or 1 to 3 carbon atoms (a “C₁-C₃ alkylene”). Examples of alkylene groups include, but are not limited to, groups such as methylene (—CH₂—), ethylene (—CH₂CH₂—), propylene (—CH₂CH₂CH₂—), butylene (—CH₂CH₂CH₂CH₂—), and the like.

“Cycloalkyl” as used herein refers to non-aromatic, saturated or unsaturated, cyclic univalent hydrocarbon structures. Particular cycloalkyl groups are those having a designated number of annular (i.e., ring) carbon atoms, for example, a cycloalkyl group having from 3 to 12 annular carbon atoms (a “C₃-C₁₂ cycloalkyl”). A particular cycloalkyl is a cyclic hydrocarbon having from 3 to 8 annular carbon atoms (a “C₃-C₈ cycloalkyl”), or having 3 to 6 annular carbon atoms (a “C₃-C₆ cycloalkyl”). Cycloalkyl can consist of one ring, such as cyclohexyl, or multiple rings, such as adamantyl, but excludes aryl (i.e., aromatic) groups. A cycloalkyl comprising more than one ring may be fused, spiro, or bridged, or combinations thereof. Examples of cycloalkyl groups include, but are not limited to, cyclopropyl

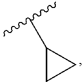 [SMILES: *C1CC1]

cyclobutyl

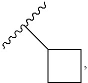 [SMILES: *C1CCC1]

cyclopentyl

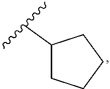 [SMILES: *C1CCCC1]

cyclohexyl

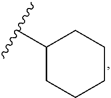 [SMILES: *C1CCCCC1]

1-cyclohexenyl, 3-cyclohexenyl, cycloheptyl

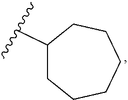 [SMILES: *C1CCCCCC1]

norbornyl, and the like.

“Cycloalkylene” as used herein refers to the same residues as cycloalkyl, but having bivalency. Particular cycloalkylene groups are those having 3 to 12 annular carbon atoms (a “C₃-C₁₂ cycloalkylene”), having from 3 to 8 annular carbon atoms (a “C₃-C₈ cycloalkylene”), or having 3 to 6 annular carbon atoms (a “C₃-C₆ cycloalkylene”). Examples of cycloalkylene groups include, but are not limited to, cyclopropylene

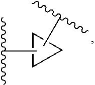

cyclopentylene

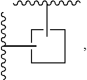

cyclopentylene

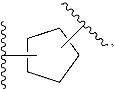

cyclohexylene

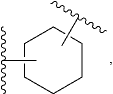

1,2-cyclohexenylene, 1,3-cyclohexenylene, 1,4-cyclohexenylene, cycloheptylene

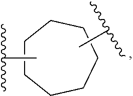

norbornylene, and the like.

“Aryl” as used herein refers to an aromatic carbocyclic group having a single ring (e.g., phenyl), or multiple condensed rings (e.g., naphthyl or anthryl) where one or more of the condensed rings may not be aromatic. Particular aryl groups are those having from 6 to 14 annular (i.e., ring) carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. Examples of aryls include, but are not limited to, groups such as phenyl, naphthyl, 1-naphthyl, 2-naphthyl, 1,2,3,4-tetrahydronaphthalen-6-yl

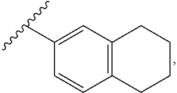 [SMILES: *c1ccc2c(c1)CCCC2]

and the like.

“Carbocyclyl” or “carbocyclic” refers to an aromatic or non-aromatic univalent cyclic group in which all of the ring members are carbon atoms, such as cyclohexyl, phenyl, 1,2-dihydronaphthyl, etc.

“Arylene” as used herein refers to the same residues as aryl, but having bivalency. Particular arylene groups are those having from 6 to 14 annular carbon atoms (a “C₆-C₁₄ arylene”). Examples of arylene include, but are not limited to, groups such as phenylene, o-phenylene (i.e., 1,2-phenylene), m-phenylene (i.e., 1,3-phenylene), p-phenylene (i.e., 1,4-phenylene), naphthylene, 1,2-naphthylene, 1,3-naphthylene, 1,4-naphthylene, 2,7-naphthylene, 2,6-naphthylene, and the like.

“Heteroaryl” as used herein refers to an unsaturated aromatic cyclic group having from 1 to 14 annular carbon atoms and at least one annular heteroatom, including, but not limited to, heteroatoms such as nitrogen (N), oxygen (O), and sulfur (S). A heteroaryl group may have a single ring (e.g., pyridyl or imidazolyl) or multiple condensed rings (e.g., indolizinyl, indolyl, or quinolinyl) where at least one of the condensed rings is aromatic. Particular heteroaryl groups are 5- to 14-membered rings having 1 to 12 annular carbon atoms and 1 to 6 annular heteroatoms independently selected from the group consisting of nitrogen (N), oxygen (O), and sulfur (S) (a “5- to 14-membered heteroaryl”); 5- to 10-membered rings having 1 to 8 annular carbon atoms and 1 to 4 annular heteroatoms independently selected from the group consisting of nitrogen, oxygen, and sulfur (a “5- to 10-membered heteroaryl”); or 5-, 6-, or 7-membered rings having 1 to 5 annular carbon atoms and 1 to 4 annular heteroatoms independently selected from the group consisting of nitrogen, oxygen, and sulfur (a “5- to 7-membered heteroaryl”). In one variation, heteroaryl includes monocyclic aromatic 5-, 6-, or 7-membered rings having from 1 to 6 annular carbon atoms and 1 to 4 annular heteroatoms independently selected from the group consisting of nitrogen, oxygen, and sulfur. In another variation, heteroaryl includes polycyclic aromatic rings having from 1 to 12 annular carbon atoms and 1 to 6 annular heteroatoms independently selected from the group consisting of nitrogen, oxygen, and sulfur. A heteroaryl group having more than one ring where at least one ring is non-aromatic may be connected to the parent structure at either an aromatic ring position or at a non-aromatic ring position. Examples of heteroaryl include, but are not limited to, groups such as pyridyl, benzimidazolyl, benzotriazolyl, benzo[b]thienyl, quinolinyl, indolyl, benzothiazolyl, and the like. “Heteroaryl” also includes moieties such as

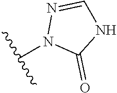 [SMILES: *n1nc[nH]c1=O]

(2,4-dihydro-3H-1,2,4-triazol-3-one-2-yl), which has the aromatic tautomeric structure

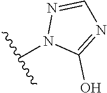 [SMILES: *n1ncnc1O]

(1H-1,2,4-triazol-5-ol-1-yl).

“Heterocyclyl” and “heterocyclic groups” as used herein refer to non-aromatic saturated or partially unsaturated cyclic groups having the number of atoms and heteroatoms as specified, or if no number of atoms or heteroatoms is specified, having at least three annular atoms, from 1 to 14 annular carbon atoms, and at least one annular heteroatom, including, but not limited to, heteroatoms such as nitrogen, oxygen, and sulfur. A heterocyclic group may have a single ring (e.g., tetrahydrothiophenyl, oxazolidinyl) or multiple condensed rings (e.g., decahydroquinolinyl, octahydrobenzo[d]oxazolyl). Multiple condensed rings include, but are not limited to, bicyclic, tricyclic, and quadracylic rings, as well as bridged or spirocyclic ring systems. Examples of heterocyclic groups include, but are not limited to, aziridinyl, azetidinyl, pyrrolidinyl, piperidinyl, oxiranyl, oxetanyl, tetrahydrofuranyl, tetrahydropyranyl, oxazolidinyl, piperazinyl, morpholinyl, dioxanyl, 3,6-dihydro-2H-pyranyl, 2,3-dihydro-1H-imidazolyl, and the like.

“Heteroarylene” as used herein refers to the same residues as heteroaryl, but having bivalency. Particular heteroarylene groups are 5- to 14-membered rings having 1 to 12 annular carbon atoms and 1 to 6 annular heteroatoms independently selected from the group consisting of nitrogen, oxygen, and sulfur (a “5- to 14-membered heteroarylene”); 5- to 10-membered rings having 1 to 8 annular carbon atoms and 1 to 4 annular heteroatoms independently selected from the group consisting of nitrogen, oxygen, and sulfur (a “5- to 10-membered heteroarylene”); or 5-, 6-, or 7-membered rings having 1 to 5 annular carbon atoms and 1 to 4 annular heteroatoms independently selected from the group consisting of nitrogen, oxygen, and sulfur (a “5- to 7-membered heteroarylene”). Examples of heteroarylene include, but are not limited to, groups such as pyridylene, benzimidazolylene, benzotriazolylene, benzo[b]thienylene, quinolinylene, indolylene, benzothiazolylene, and the like.

“Halo” or “halogen” refers to elements of the Group 17 series having atomic number 9 to 85. Halo groups include fluoro (F), chloro (Cl), bromo (Br), and iodo (I).

“Haloalkyl,” “haloalkylene,” “haloaryl,” “haloarylene,” “haloheteroaryl,” and similar terms refer to a moiety substituted with at least one halo group. Where a haloalkyl moiety or other halo-substituted moiety is substituted with more than one halogen, it may be referred to by using a prefix corresponding to the number of halogen moieties attached. For example, dihaloaryl, dihaloalkyl, trihaloaryl, trihaloalkyl, etc., refer to aryl and alkyl substituted with two (“di”) or three (“tri”) halo groups, which may be, but are not necessarily, the same halo; thus, for example, the haloaryl group 4-chloro-3-fluorophenyl is within the scope of dihaloaryl. The subset of haloalkyl groups in which each hydrogen (H) of an alkyl group is replaced with a halo group is referred to as a “perhaloalkyl.” A particular perhaloalkyl group is trifluoroalkyl (—CF₃). Similarly, “perhaloalkoxy” refers to an alkoxy group in which a halogen takes the place of each hydrogen (H) in the hydrocarbon making up the alkyl moiety of the alkoxy group. An example of a perhaloalkoxy group is trifluoromethoxy (—OCF₃). “Haloalkyl” includes monohaloalkyl, dihaloalkyl, trihaloalkyl, perhaloalkyl, and any other number of halo substituents possible on an alkyl group; and similarly for other groups such as haloalkylene, haloaryl, haloarylene, haloheteroaryl, etc.

“Amino” refers to the group —NH₂.

“Oxo” refers to the group ═O, that is, an oxygen atom doubly bonded to carbon or another chemical element.

“Optionally substituted,” unless otherwise specified, means that a group is unsubstituted or substituted by one or more (e.g., 1, 2, 3, 4, or 5) of the substituents listed for that group, in which the substituents may be the same or different. In one embodiment, an optionally substituted group is unsubstituted. In one embodiment, an optionally substituted group has one substituent. In another embodiment, an optionally substituted group has two substituents. In another embodiment, an optionally substituted group has three substituents. In another embodiment, an optionally substituted group has four substituents. In certain embodiments, an optionally substituted group has 1 to 2, 1 to 3, 1 to 4, or 1 to 5 substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

In addition to the disclosure herein, the term “substituted,” when used to modify a specified group or radical, can also mean that one or more hydrogen atoms (H) of the specified group or radical are each, independently of one another, replaced with the same or different substituent groups as defined herein. In certain embodiments, a group that is substituted has 1, 2, 3, or 4 substituents, 1, 2, or 3 substituents, 1 or 2 substituents, or one substituent.

Substituents can be attached to any chemically possible location on the specified group or radical, unless indicated otherwise. Thus, in one embodiment, —C₁-C₈ alkyl-OH includes, for example, —CH₂CH₂OH, —CH(OH)—CH₃, —CH₂C(OH)(CH₃)₂, and the like. By way of further example, in one embodiment, —C₁-C₆ alkyl-OH includes, for example, —CH₂CH₂OH, —CH(OH)—CH₃, —CH₂C(OH)(CH₃)₂, and the like. By way of further example, in one embodiment, —C₁-C₆ alkyl-CN includes, for example, —CH₂CH₂CN, —CH(CN)—CH₃, —CH₂C(CN)(CH₃)₂, and the like.

Unless a specific isotope of an element is indicated in a formula, the disclosure includes all isotopologues of the compounds disclosed herein, such as, for example, deuterated derivatives of the compounds (where H can be 2H, i.e., deuterium (D)). Deuterated compounds may provide favorable changes in pharmacokinetic (ADME) properties. Isotopologues can have isotopic replacements at any or at all locations in a structure, or can have atoms present in natural abundance at any or all locations in a structure.

A “small molecule” as used herein refers to a compound of 1,000 daltons or less in molecular weight.

Hydrogen atoms can also be replaced with close bioisosteres, such as fluorine, provided that such replacements result in stable compounds.

The disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by the disclosure, as well as the general non-stereospecific form and mixtures of the disclosed compounds in any ratio, including mixtures of two or more stereochemical forms of a disclosed compound in any ratio, such that racemic, non-racemic, enantioenriched, and scalemic mixtures of a compound are embraced. Compositions comprising a disclosed compound also are intended, such as a composition of a substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched, and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

This disclosure also embraces any and all tautomeric forms of the compounds described herein.

The disclosure is intended to embrace all salts of the compounds described herein, as well as methods of using such salts of the compounds. In one embodiment, the salts of the compounds comprise pharmaceutically acceptable salts. Pharmaceutically acceptable salts are those salts that can be administered as drugs or pharmaceuticals to humans and/or animals and that, upon administration, retain at least some of the biological activity of the free compound (i.e., neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-906-39051-2). Several pharmaceutically acceptable salts are also disclosed in Berge, J. Pharm. Sci. 66(1):1-19 (1977).

### 2. CBL Biomarkers

In one aspect, provided herein are methods of detecting proximal biomarkers of CBL inhibition in a sample. In certain embodiments, embodiments, the methods comprise the steps of measuring the amount of a proximal biomarker of CBL inhibition in the sample.

The CBL biomarker can be any CBL biomarker deemed suitable by the person of skill. In certain embodiments, the CBL biomarker is a biomarker that correlates with CBL levels in vivo. In certain embodiments, the CBL biomarker is a biomarker that correlates with CBL expression in vivo. In certain embodiments, the CBL biomarker is a biomarker that correlates with CBL activity in vivo. In certain embodiments, the CBL biomarker is a biomarker that correlates with CBL protein levels in vivo. In certain embodiments the CBL biomarker is directly proportional to CBL level in vivo. In certain embodiments, the CBL biomarker is a biomarker that is directly proportional to CBL level in vivo to within a useful error of measurement.

In certain embodiments, the biomarker is selected from pITK, pHS1, pPLCγ2, pPLCγ1, pSYK, pZAP70, and combinations thereof. In certain embodiments, the biomarker is two of pITK, pHS1, pPLCγ2, pPLCγ1, pSYK, and pZAP70. In certain embodiments, the biomarker is three of pITK, pHS1, pPLCγ2, pPLCγ1, pSYK, and pZAP70. In certain embodiments, the biomarker is four of pITK, pHS1, pPLCγ2, pPLCγ1, pSYK, and pZAP70. In certain embodiments, the biomarker is five of pITK, pHS1, pPLCγ2, pPLCγ1, pSYK, and pZAP70. In certain embodiments, the biomarker is six of pITK, pHS1, pPLCγ2, pPLCγ1, pSYK, and pZAP70. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70. In certain embodiments, the biomarker is pHS1, PLCγ2, and pZAP70. In other words, in certain embodiments, pHS1, pPLCγ2, and pZAP70 are measured to assess CBL level in vivo.

In certain embodiments, the methods detect CBL level in the sample. In certain embodiments, the methods detect CBL expression in the sample. In certain embodiments, the methods detect CBL activity in the sample. In certain embodiments, the methods detect CBL protein amount in the sample. In certain embodiments, the methods detect inhibition of CBL level in the sample. In certain embodiments, the methods detect inhibition of CBL expression in the sample. In certain embodiments, the methods detect inhibition of CBL activity in the sample. In certain embodiments, the methods detect inhibition of CBL protein amount in the sample. In certain embodiments, the methods detect degradation of CBL in the sample.

In certain embodiments, the sample is from a mammal. In certain embodiments, the sample is from a non-human mammal. In certain embodiments, the sample is from a human. In certain embodiments, the sample is from a human in need of treatment for a disease or disorder treatable with a CBL inhibitor. In certain embodiments, the sample is a tissue sample. In certain embodiments, the sample is a cell sample. In certain embodiments, the sample is a blood sample. In certain embodiments, the sample is a plasma sample. In certain embodiments, the sample is a tumor sample. In certain embodiments, the sample is a tumor biopsy. In certain embodiments, the sample is a cell culture.

The CBL can be any CBL deemed suitable to the person of skill. In certain embodiments, the CBL is Cbl-b. In certain embodiments, the CBL is c-Cbl. In certain embodiments, the CBL is Cbl-b and c-Cbl. In other words, in certain embodiments, the biomarker is useful for assessing Cbl-b and c-Cbl levels in vivo.

In another aspect, provided herein are methods for detecting the amount of CBL in a cell, tissue, or organism. In certain embodiments, the methods comprise the steps of measuring the amount of a CBL biomarker in a first sample of the cell, tissue, or organism; measuring the amount of a CBL biomarker in a second sample of the cell, tissue, or organism; and identifying a change in the amount of CBL in the cell, tissue, or organism based on the change in the amount of the CBL biomarker detected in the sample.

The CBL biomarker, sample, and CBL are as described above. In certain embodiments, the first sample is obtained prior to administration of a CBL inhibitor. In certain embodiments, the first sample is obtained at the first administration of a CBL inhibitor. In certain embodiments, the first sample is obtained prior to administration of a CBL inhibitor, and the second sample is obtained at the first administration of a CBL inhibitor. In certain embodiments, the first sample is obtained at the first administration of a CBL inhibitor, and the second sample is obtained after the first administration of a CBL inhibitor. In certain embodiments, the measuring steps are repeated 1, 2, 3, 4, 5, 10, 15, 20, 25, or more times.

In another aspect, provided herein are methods of treating a patient in need thereof with a CBL inhibitor. In certain embodiments, the methods comprise administering to a patient a first dose of a CBL inhibitor; measuring the amount of a CBL biomarker in a first sample of the patient; and identifying the amount of CBL in the patient based on the amount of the CBL biomarker detected. In certain embodiments, the CBL is Cbl-b. In certain embodiments, the CBL is c-Cbl. In certain embodiments, the CBL is Cbl-b and c-Cbl.

The CBL biomarker, sample, and CBL are as described above. The CBL inhibitor can be any CBL inhibitor deemed suitable by the person of skill. The treatment can be a treatment of any CBL disease or disorder described herein. In certain embodiments, the CBL inhibitor is a CBL inhibitor described herein. In certain embodiments, the CBL inhibitor is Compound 23, described herein. In certain embodiments, a subsequent dose of the CBL inhibitor is adjusted or determined based on the measured CBL level. If the CBL level is higher than desired, a higher subsequent dose of the CBL inhibitor can be selected. If the CBL level is lower than desired, a lower subsequent dose of the CBL inhibitor can be selected. In certain embodiments, the measuring steps are repeated 1, 2, 3, 4, 5, 10, 15, 20, 25, or more times. In certain embodiments, the administering steps are repeated 1, 2, 3, 4, 5, 10, 15, 20, 25, or more times. In certain embodiments, the measuring and administering steps are repeated 1, 2, 3, 4, 5, 10, 15, 20, 25, or more times.

In another aspect, provided herein are methods of treating a patient in need thereof with a CBL inhibitor. In certain embodiments, the methods comprise administering to a patient a first dose of a CBL inhibitor; measuring the amount of a CBL biomarker in a first sample of the cell, tissue, or organism; measuring the amount of a CBL biomarker in a second sample of the cell, tissue, or organism; and identifying a change in the amount of CBL in the cell, tissue, or organism based on the change in the amount of the CBL biomarker detected in the sample. In certain embodiments, the CBL is Cbl-b. In certain embodiments, the CBL is c-Cbl. In certain embodiments, the CBL is Cbl-b and c-Cbl. In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70. In certain embodiments, the biomarker is selected from pITK, pHS1, pPLCγ2, pPLCγ1, pSYK, pZAP70, and combinations thereof. In certain embodiments, the biomarker is two of pITK, pHS1, pPLCγ2, pPLCγ1, pSYK, and pZAP70. In certain embodiments, the biomarker is three of pITK, pHS1, pPLCγ2, pPLCγ1, pSYK, and pZAP70. In certain embodiments, the biomarker is four of pITK, pHS1, pPLCγ2, pPLCγ1, pSYK, and pZAP70. In certain embodiments, the biomarker is five of pITK, pHS1, pPLCγ2, pPLCγ1, pSYK, and pZAP70. In certain embodiments, the biomarker is six of pITK, pHS1, pPLCγ2, pPLCγ1, pSYK, and pZAP70.

The CBL biomarker, sample, CBL, CBL inhibitor, and CBL disease or disorder are as described above. In certain embodiments, a subsequent dose of the CBL inhibitor is adjusted or determined based on the measured CBL level. If the CBL level is higher than desired, a higher subsequent dose of the CBL inhibitor can be selected. If the CBL level is lower than desired, a lower subsequent dose of the CBL inhibitor can be selected. In certain embodiments, the measuring steps are repeated 1, 2, 3, 4, 5, 10, 15, 20, 25, or more times. In certain embodiments, the administering steps are repeated 1, 2, 3, 4, 5, 10, 15, 20, 25, or more times. In certain embodiments, the measuring and administering steps are repeated 1, 2, 3, 4, 5, 10, 15, 20, 25, or more times.

In certain embodiments the methods further comprise administering a second dose of the CBL inhibitor, wherein the dose is selected based on the amount of CBL identified. In certain embodiments of the methods, the second dose is increased if the amount of the CBL biomarker in a sample is below a predetermined range; wherein the second dose is maintained if the amount of the CBL biomarker in a sample is within a predetermined range; and/or wherein the second dose is decreased if the amount of the CBL biomarker in a sample is above a predetermined range. In certain embodiments of the methods, the second dose is decreased if the amount of the CBL biomarker in a sample is below a predetermined range; wherein the second dose is maintained if the amount of the CBL biomarker in a sample is within a predetermined range; and/or wherein the second dose is increased if the amount of the CBL biomarker in a sample is above a predetermined range. In certain embodiments of the methods, the second dose is increased, decreased, or maintained relative to the first dose.

In certain embodiments of the methods, an increase in the amount of the CBL biomarker indicates a decrease in the amount of CBL. In certain embodiments of the methods, a decrease in the amount of the CBL biomarker indicates a decrease in the amount of CBL.

In certain embodiments, the methods are conducted in vitro. In certain embodiments, the methods are conducted ex vivo.

The CBL biomarker can be detected by any technique deemed suitable. In certain embodiments, the CBL biomarker is detected by immunoassay, western blotting, ELISA, mass spectrometry, immunohistochemistry or flow cytometry. In certain embodiments, proximal biomarkers of CBL inhibition is detected by flow cytometry.

The disease or condition can be any disease or condition deemed suitable to the person of skill. In certain embodiments, the disease or condition is a cancer. In certain embodiments, the disease or condition is a solid tumor. In certain embodiments, the disease or condition is a hematological cancer.

In particular embodiments, the CBL biomarker is selected from phosphorylated interleukin-2-inducible kinase (pITK), phosphorylated hematopoietic lineage cell-specific protein (pHS1), phosphorylated phospholipase C2 (pPLCγ2), phosphorylated phospholipase C1 (pPLCγ1), phosphorylated spleen tyrosine kinase (pSYK), and phosphorylated Zeta chain associated protein kinase 70 (pZAP70). In certain embodiments, the biomarker is selected from pITK, pHS1, pPLCγ2, pPLCγ1, pSYK, pZAP70, and combinations thereof. In certain embodiments, the biomarker is pITK. In certain embodiments, the biomarker is pHS1. In certain embodiments, the biomarker is pPLCγ2. In certain embodiments, the biomarker is pPLCγ1. In certain embodiments, the biomarker is pSYK. In certain embodiments, the biomarker is pZAP70. In certain embodiments, the biomarker is pHS1 and pPLCγ2. In certain embodiments, the biomarker is pPLCγ2 and pZAP70. In certain embodiments, the biomarker is pHS1 and pZAP70. In certain embodiments, the biomarker is pHS1, pPLCγ2, and pZAP70. In certain embodiments, the biomarker is two of pITK, pHS1, pPLCγ2, pPLCγ1, pSYK, and pZAP70. In certain embodiments, the biomarker is three of pITK, pHS1, pPLCγ2, pPLCγ1, pSYK, and pZAP70. In certain embodiments, the biomarker is four of pITK, pHS1, pPLCγ2, pPLCγ1, pSYK, and pZAP70. In certain embodiments, the biomarker is five of pITK, pHS1, pPLCγ2, pPLCγ1, pSYK, and pZAP70. In certain embodiments, the biomarker is six of pITK, pHS1, pPLCγ2, pPLCγ1, pSYK, and pZAP70.

### 3. CBL Compounds

In the methods and compositions, the CBL compound can be any CBL compound deemed suitable to the person of skill. In certain embodiments, the CBL compound is any compound described in WO 2006/136502, WO 2007/010217, WO 2008/070305, WO 2019/148005, WO 2020/210508, WO 2020/236654, WO 2020/264398, or WO 2021/021761, WO 2021/243471, WO 2022/169997, WO 2022/169998, WO 2022/217276, WO 2022/221704, WO 2022/272248, or WO 2023/036330, the contents of which are hereby incorporated by reference in their entireties.

The CBL inhibitor can be administered or delivered by any composition or means deemed suitable to the practitioner of skill. In certain embodiments, the CBL inhibitor is a small molecule, administered in a pharmaceutical composition. In certain embodiments, the CBL inhibitor is delivered by an antibody, for instance as an antibody-drug conjugate. In certain embodiments, the CBL inhibitor is delivered in a delivery vehicle.

In certain embodiments, the CBL inhibitor compound is a compound of Formula (I):

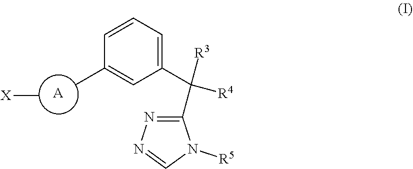

or a tautomer thereof, or a pharmaceutically acceptable salt thereof,
wherein:

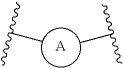

is

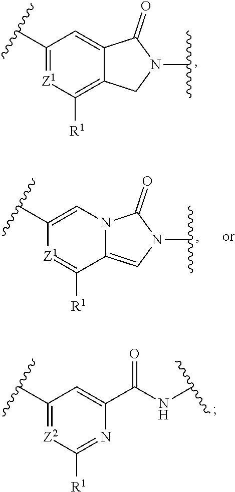

Z¹ is CH or N;
Z² is CH or N;
R¹ is —CF₃ or cyclopropyl;
R² is —CF₃ or cyclopropyl;
R³ is H, C₁-C₂ alkyl, or C₁-C₂ haloalkyl;
R⁴ is H, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups;
or R³ and R⁴ are taken together with the carbon atom to which they are attached to form C₃-C₅ cycloalkyl or 4- to 6-membered heterocyclyl, each of which is optionally substituted by 1-5 R⁶ groups;
R⁵ is H, C₁-C₆ alkyl, C₁-C₆ haloalkyl, or C₃-C₆ cycloalkyl;
each R⁶ is independently C₁-C₆ alkyl, halo, hydroxy, —O—(C₁-C₆ alkyl), —CN, C₁-C₆ alkyl-CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or spiro 4- to 6-membered heterocyclyl; X is H, C₁-C₆ alkyl, C₁-C₆ haloalkyl, C₁-C₆ alkyl-OH, C₁-C₆ alkyl-CN, C₃-C₆ cycloalkyl optionally substituted by 1-5 R⁸ groups, or

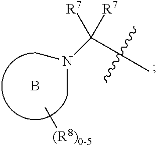

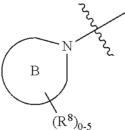

is 4- to 7-membered heterocyclyl or 5- to 8-membered heteroaryl, each of which heterocyclyl or heteroaryl optionally contains 1-2 additional heteroatoms selected from the group consisting of N, O, and S, and each of which heterocyclyl or heteroaryl is optionally substituted by 1-5 R⁸ groups;
each R⁷ is independently H, C₁-C₆ alkyl, C₁-C₆ alkyl-OH, or C₁-C₆ haloalkyl;
or two R⁷ groups are taken together with the carbon atom to which they are attached to form a C₃-C₅ cycloalkyl or 3- to 5-membered heterocyclyl; and
each R⁸ is independently halo, C₁-C₆ alkyl, C₁-C₆ alkyl-CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

In certain embodiments,

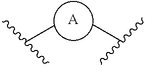

(i.e., the Ring A moiety), is

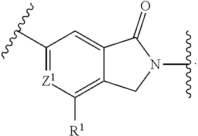

In certain embodiments,

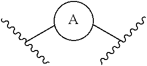

(i.e., the Ring A moiety), is

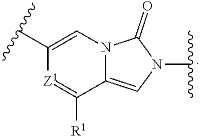

In certain embodiments, Z¹ is CH. In other embodiments, Z¹ is N. In certain embodiments, R¹ is —CF₃. In other embodiments, R¹ is cyclopropyl. In certain embodiments, the Ring A moiety is

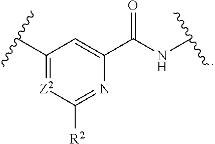

In certain embodiments, Z² is CH. In other embodiments, Z² is N. In certain embodiments, R² is —CF₃. In other embodiments, R² is cyclopropyl. In certain embodiments, the Ring A moiety is selected from the group consisting of:

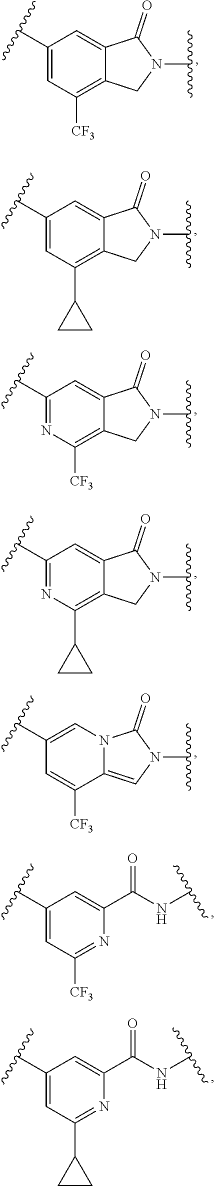

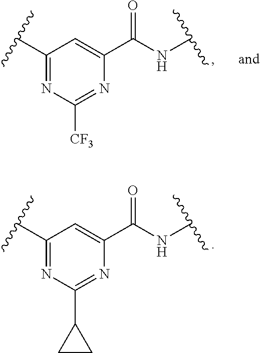 [SMILES: *NC(=O)c1cc(*)nc(C(F)(F)F)n1 ; *NC(=O)c1cc(*)nc(C2CC2)n1]

In certain embodiments, the Ring A moiety is

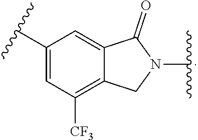 [SMILES: *c1cc2c(c(C(F)(F)F)c1)CN(*)C2=O]

In certain embodiments, the Ring A moiety is

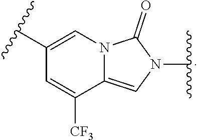 [SMILES: *c1cc(C(F)(F)F)c2cn(*)c(=O)n2c1]

In certain embodiments, the Ring A moiety is

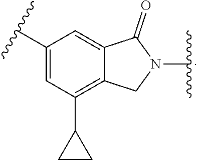 [SMILES: *c1cc2c(c(C3CC3)c1)CN(*)C2=O]

In certain embodiments, the Ring A moiety is

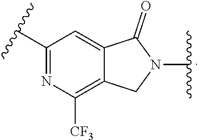 [SMILES: *c1cc2c(c(C(F)(F)F)n1)CN(*)C2=O]

In certain embodiments, the Ring A moiety is

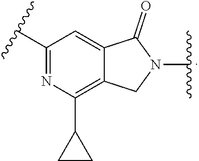 [SMILES: *c1cc2c(c(C3CC3)n1)CN(*)C2=O]

In certain embodiments, the Ring A moiety is

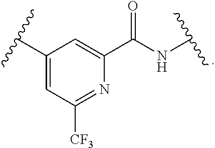 [SMILES: *NC(=O)c1cc(*)cc(C(F)(F)F)n1]

In certain embodiments, the Ring A moiety is

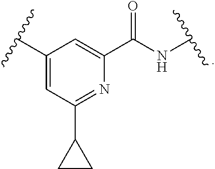 [SMILES: *NC(=O)c1cc(*)cc(C2CC2)n1]

In certain embodiments, the Ring A moiety is

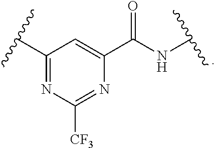 [SMILES: *NC(=O)c1cc(*)nc(C(F)(F)F)n1]

In certain embodiments, the Ring A moiety is

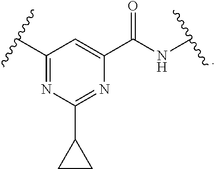 [SMILES: *NC(=O)c1cc(*)nc(C2CC2)n1]

In certain embodiments, R³ is H, C₁-C₂ alkyl, or C₁-C₂ haloalkyl. In certain embodiments, R³ is H, —CH₃, or —CF₃.

In certain embodiments, R³ is H.

In certain embodiments, R³ is C₁-C₂ alkyl. In certain embodiments, R³ is methyl. In certain embodiments, R³ is ethyl.

In certain embodiments, R³ is C₁-C₂ haloalkyl. In certain embodiments, R³ is C₁-C₂ haloalkyl containing 1-5 halogen atoms. In certain embodiments, R³ is C₁-C₂ haloalkyl containing 1-3 halogen atoms. In certain embodiments, R³ is C₁ haloalkyl. In certain embodiments, R³ is C₂ haloalkyl. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R³ is —CF₃, —CCl₃, —CF₂Cl, —CFCl₂, —CHF₂, —CH₂F, —CHCl₂, —CH₂Cl, or —CHFCl. In certain embodiments, R³ is —CF₃.

In certain embodiments, R⁴ is H, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups. In certain embodiments, R⁴ is H, C₁-C₃ alkyl, C₁-C₃ haloalkyl, 4- to 6-membered heterocyclyl, or C₄-C₅ cycloalkyl, wherein the heterocyclyl or cycloalkyl groups are optionally substituted by 1-3 R⁶ groups. In certain embodiments, R⁴ is H, —CH₃, —CF₃, cyclobutyl, or

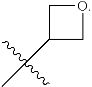 [SMILES: *C1COC1]

In certain embodiments, R⁴ is H.

In certain embodiments, R⁴ is C₁-C₆ alkyl. In certain embodiments, R⁴ is C₁-C₃ alkyl. In certain embodiments, R⁴ is methyl, ethyl, n-propyl, or isopropyl. In certain embodiments, R⁴ is —CH₃.

In certain embodiments, R⁴ is C₁-C₆ haloalkyl. In certain embodiments, R⁴ is C₁-C₆ haloalkyl containing 1-7 halogen atoms. In certain embodiments, R⁴ is C₁-C₃ haloalkyl. In certain embodiments, R⁴ is C₁-C₃ haloalkyl containing 1-7 halogen atoms. In certain embodiments, R⁴ is C₁-C₂ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁴ is —CF₃, —CCl₃, —CF₂Cl, —CFCl₂, —CHF₂, —CH₂F, —CHCl₂, —CH₂F, or —CHFCl. In certain embodiments, R⁴ is —CF₃.

In certain embodiments, R⁴ is 4- to 8-membered heterocyclyl optionally substituted by 1-5 R⁶ groups. In certain embodiments, R⁴ is 4- to 6-membered heterocyclyl optionally substituted by 1-3 R⁶ groups. In certain embodiments, R⁴ is a 4-membered heterocyclyl optionally substituted by 1-2 R⁶ groups. In certain embodiments, the heterocyclyl is substituted by 5 R⁶ groups. In certain embodiments, the heterocyclyl is substituted by 4 R⁶ groups. In certain embodiments, the heterocyclyl is substituted by 3 R⁶ groups. In certain embodiments, the heterocyclyl is substituted by 2 R⁶ groups. In certain embodiments, the heterocyclyl is substituted by 1 R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R⁴ is:

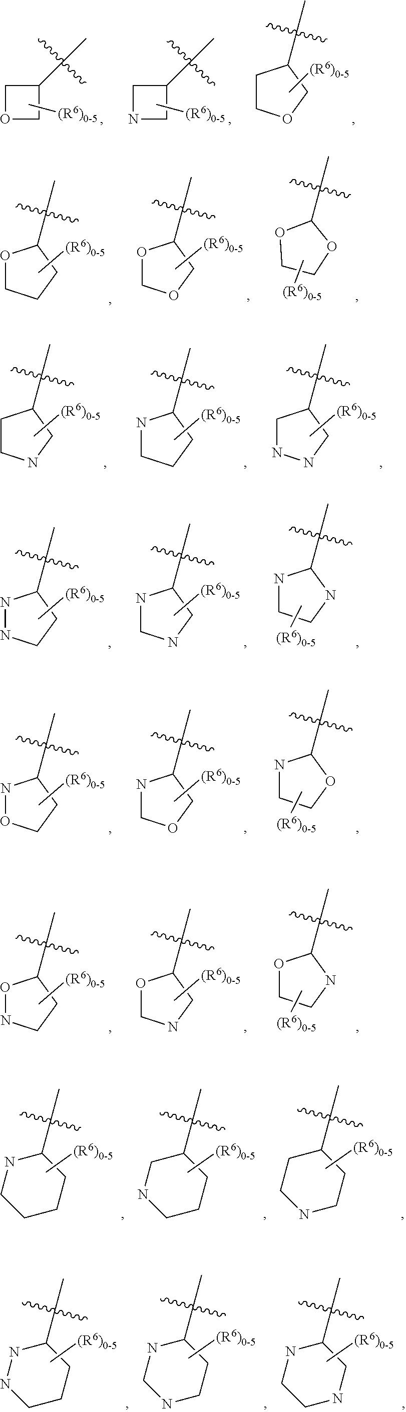

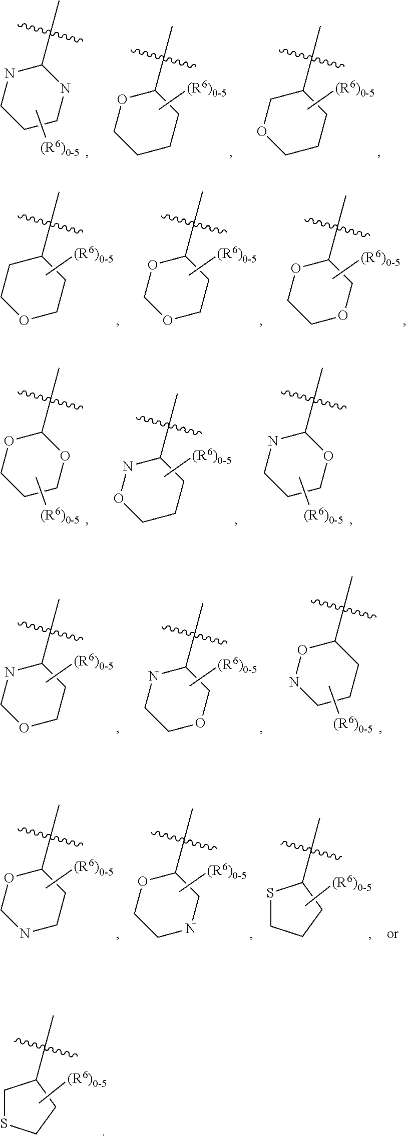

In certain embodiments, R⁴ is

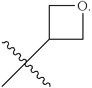 [SMILES: *C1COC1]

In certain embodiments, R⁴ is C₃-C₆ cycloalkyl optionally substituted by 1-5 R⁶ groups. In certain embodiments, R⁴ is C₄-C₅ cycloalkyl optionally substituted by 1-3 R⁶ groups. In certain embodiments, the cycloalkyl is substituted by 5 R⁶ groups. In certain embodiments, the cycloalkyl is substituted by 4 R⁶ groups. In certain embodiments, the cycloalkyl is substituted by 3 R⁶ groups. In certain embodiments, the cycloalkyl is substituted by 2 R⁶ groups. In certain embodiments, the cycloalkyl is substituted by 1 R⁶ group. In certain embodiments, the cycloalkyl is unsubstituted. In certain embodiments, R⁴ is cyclopropyl, cyclobutyl, cyclopentyl, or cyclohexyl, each of which is optionally substituted by 1-5 R⁶ groups. In certain embodiments, R⁴ is cyclopropyl or cyclobutyl. In certain embodiments, R⁴ is cyclobutyl.

In certain embodiments, R³ and R⁴ are taken together with the carbon atom to which they are attached to form C₃-C₈ cycloalkyl or 4- to 6-membered heterocyclyl, each of which is optionally substituted by 1-5 R⁶ groups. In certain embodiments, R³ and R⁴ are taken together with the carbon atom to which they are attached to form C₄-C₅ cycloalkyl or 4- to 6-membered heterocyclyl, each of which is optionally substituted by 1-3 R⁶ groups. In certain embodiments, R³ and R⁴ are taken together with the carbon atom to which they are attached to form

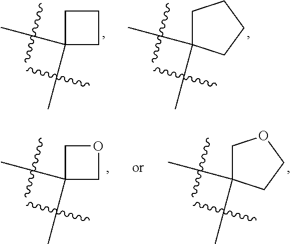 [SMILES: *C1(*)CCC1 ; *C1(*)CCCC1 ; *C1(*)COC1 ; *C1(*)CCOC1]

each of which is optionally substituted by 1-3 R⁶ groups. In certain embodiments, R³ and R⁴ are taken together with the carbon atom to which they are attached, and are substituted by 1 R⁶ group which is methyl, to form

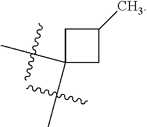 [SMILES: *C1(*)CC(C)C1]

In certain embodiments, R³ and R⁴ are taken together with the carbon atom to which they are attached to form C₃-C₅ cycloalkyl optionally substituted by 1-5 R⁶ groups. In certain embodiments, R³ and R⁴ are taken together with the carbon atom to which they are attached to form C₄-C₅ cycloalkyl optionally substituted by 1-3 R⁶ groups. In certain embodiments, the cycloalkyl is substituted by 5 R⁶ groups. In certain embodiments, the cycloalkyl is substituted by 4 R⁶ groups. In certain embodiments, the cycloalkyl is substituted by 3 R⁶ groups. In certain embodiments, the cycloalkyl is substituted by 2 R⁶ groups. In certain embodiments, the cycloalkyl is substituted by 1 R⁶ group. In certain embodiments, the cycloalkyl is unsubstituted. In certain embodiments, R³ and R⁴ are taken together with the carbon atom to which they are attached to form

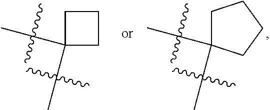 [SMILES: *C1(*)CCC1 ; *C1(*)CCCC1]

each of which is optionally substituted by 1-3 R⁶ groups. In certain embodiments, R³ and R⁴ are taken together with the carbon atom to which they are attached, and are substituted by 1 R⁶ group which is methyl, to form

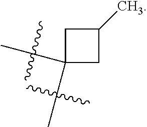 [SMILES: *C1(*)CC(C)C1]

In certain embodiments, the absolute stereochemistry at the carbon atom to which the methyl group of

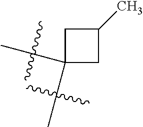 [SMILES: *C1(*)CC(C)C1]

is attached is (R)—(using the Cahn-Ingold-Prelog rules). In certain embodiments, the absolute stereochemistry at the carbon atom to which the methyl group of

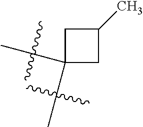 [SMILES: *C1(*)CC(C)C1]

is attached is (S)—.

In certain embodiments, R³ and R⁴ are taken together with the carbon atom to which they are attached to form 4- to 6-membered heterocyclyl optionally substituted by 1-5 R⁶ groups. In certain embodiments, R³ and R⁴ are taken together with the carbon atom to which they are attached to form 4- to 6-membered heterocyclyl optionally substituted by 1-3 R⁶ groups. In certain embodiments, the heterocyclyl is substituted by 5 R⁶ groups. In certain embodiments, the heterocyclyl is substituted by 4 R⁶ groups. In certain embodiments, the heterocyclyl is substituted by 3 R⁶ groups. In certain embodiments, the heterocyclyl is substituted by 2 R⁶ groups. In certain embodiments, the heterocyclyl is substituted by 1 R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R³ and R⁴ are taken together with the carbon atom to which they are attached to form

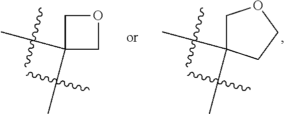 [SMILES: *C1(*)COC1 ; *C1(*)CCOC1]

each of which is optionally substituted by 1-3 R⁶ groups. In certain embodiments, R³ and R⁴ are taken together with the carbon atom to which they are attached to form

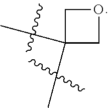 [SMILES: *C1(*)COC1]

In certain embodiments, R³ and R⁴ are taken together with the carbon atom to which they are attached to form

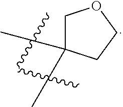

In certain embodiments, each R⁶ is independently C₁-C₆ alkyl, halo, hydroxy, —O—(C₁-C₆ alkyl), —CN, C₁-C₆ alkyl-CN, C₁-C₆ alkyl-OH, or C₁-C₆ haloalkyl. In certain embodiments, each R⁶ is independently C₁-C₃ alkyl, halo, hydroxy, —O—(C₁-C₃ alkyl), —CN, C₁-C₃ alkyl-CN, C₁-C₃ alkyl-OH, or C₁-C₃ haloalkyl. In certain embodiments, each R⁶ is independently —CH₃, fluoro (F), hydroxy, —OCH₃, —CN, —CH₂CN, —CH₂OH, or —CF₃.

In certain embodiments, R⁶ is C₁-C₆ alkyl. In certain embodiments, R⁶ is C₁-C₃ alkyl. In certain embodiments, R⁶ is methyl, ethyl, n-propyl, or isopropyl. In certain embodiments, R⁶ is —CH₃.

In certain embodiments, R⁶ is halo. In certain embodiments, R⁶ is chloro, fluoro, or bromo. In certain embodiments, R⁶ is chloro or fluoro. In certain embodiments, R⁷ is fluoro.

In certain embodiments, R⁶ is hydroxyl.

In certain embodiments, R⁶ is —O(C₁-C₆ alkyl). In certain embodiments, R⁶ is —O—(C₁-C₃ alkyl). In certain embodiments, R⁶ is —O(methyl), —O(ethyl), —O(n-propyl), or —O(isopropyl). In certain embodiments, R⁶ is —OCH₃ or —OCH₂CH₃. In certain embodiments, R⁶ is —OCH₃.

In certain embodiments, R⁶ is —CN. In certain embodiments, R⁶ is C₁-C₆ alkyl-CN. In certain embodiments, R⁶ is C₁-C₃ alkyl-CN. In certain embodiments, R⁶ is —CH₂CN, —CH₂CH₂—CN, —CH₂CH₂CH₂—CN, or —C(CH₃)₂—CN. In certain embodiments, R⁶ is —CH₂CN.

In certain embodiments, R⁶ is C₁-C₆ alkyl-OH. In certain embodiments, R⁶ is C₁-C₃ alkyl-OH. In certain embodiments, R⁶ is —CH₂OH, —CH₂CH₂—OH, —CH₂CH₂CH₂—OH, or —C(CH₃)₂—OH. In certain embodiments, R⁶ is —CH₂OH.

In certain embodiments, R⁶ is C₁-C₆ haloalkyl. In certain embodiments, R⁶ is C₁-C₆ haloalkyl containing 1-7 halogen atoms. In certain embodiments, R⁶ is C₁-C₃ haloalkyl. In certain embodiments, R⁶ is C₁-C₃ haloalkyl containing 1-7 halogen atoms. In certain embodiments, R⁶ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁶ is —CF₃, —CCl₃, —CF₂Cl, —CFCl₂, —CHF₂, —CH₂F, —CHCl₂, —CH₂Cl, or —CHFCl. In certain embodiments, R⁶ is —CF₃.

In certain embodiments, two R⁶ groups attached to the same carbon atom are taken together with the carbon atom to which they are attached to form a spiro C₃-C₆ cycloalkyl or spiro 4- to 6-membered heterocyclyl. In certain embodiments, two R⁶ groups attached to the same carbon atom are taken together with the carbon atom to which they are attached to form a spiro C₃-C₆ cycloalkyl or spiro 4- to 5-membered heterocyclyl.

In certain embodiments, two R⁶ groups attached to the same carbon atom are taken together with the carbon atom to which they are attached to form a spiro C₃-C₆ cycloalkyl. In certain embodiments, two R⁶ groups attached to the same carbon atom are taken together with the carbon atom to which they are attached to form a spiro C₃-C₅ cycloalkyl. In certain embodiments, two R⁶ groups attached to the same carbon atom are taken together with the carbon atom to which they are attached to form a spiro C₃-C₄ cycloalkyl. In certain embodiments, two R⁶ groups attached to the same carbon atom are taken together with the carbon atom to which they are attached to form a spiro cyclopropyl, cyclobutyl, or cyclopentyl. In certain embodiments, two R⁶ groups attached to the same carbon atom are taken together with the carbon atom to which they are attached to form a spiro cyclopropyl.

In certain embodiments, two R⁶ groups attached to the same carbon atom are taken together with the carbon atom to which they are attached to form a spiro 4- to 6-membered heterocyclyl. In certain embodiments, two R⁶ groups attached to the same carbon atom are taken together with the carbon atom to which they are attached to form a spiro 4- to 5-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁶ groups attached to the same carbon atom are taken together with the carbon atom to which they are attached to form a spiro oxetanyl, azetidinyl, tetrahydrofuranyl, dioxolanyl, pyrrolidinyl, pyrazolidinyl, piperidinyl, isoxazolidinyl, or tetrahydropyranyl.

In certain embodiments, R⁵ is H, C₁-C₆ alkyl, C₁-C₆ haloalkyl, or C₃-C₆ cycloalkyl. In certain embodiments, R⁵ is H, C₁-C₃ alkyl, C₁-C₃ haloalkyl, or C₃-C₄ cycloalkyl. In certain embodiments, R⁵ is H, —CH₃, —CHF₂, or cyclopropyl.

In certain embodiments, R⁵ is H.

In certain embodiments, R⁵ is C₁-C₆ alkyl. In certain embodiments, R⁵ is C₁-C₃ alkyl. In certain embodiments, R⁵ is methyl, ethyl, n-propyl, or isopropyl. In certain embodiments, R⁵ is —CH₃.

In certain embodiments, R⁵ is C₁-C₆ haloalkyl. In certain embodiments, R⁵ is C₁-C₆ haloalkyl containing 1-7 halogen atoms. In certain embodiments, R⁵ is C₁-C₃ haloalkyl. In certain embodiments, R⁵ is C₁-C₃ haloalkyl containing 1-7 halogen atoms. In certain embodiments, R⁵ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁵ is —CF₃, —CCl₃, —CF₂Cl, —CFCl₂, —CHF₂, —CH₂F, —CHCl₂, —CH₂Cl, or —CHFCl. In certain embodiments, R⁵ is —CHF₂.

In certain embodiments, R⁵ is C₃-C₆ cycloalkyl. In certain embodiments, R⁵ is C₃-C₅ cycloalkyl. In certain embodiments, R⁵ is C₃-C₄ cycloalkyl. In certain embodiments, R⁵ is cyclopropyl, cyclobutyl, or cyclopentyl. In certain embodiments, R⁵ is cyclopropyl.

In certain embodiments, X is H, C₁-C₆ alkyl, C₁-C₆ haloalkyl, C₁-C₆ alkyl-OH, C₁-C₆ alkyl-CN, or C₃-C₆ cycloalkyl optionally substituted by 1-5 R⁸ groups. In certain embodiments, X is H, C₁-C₃ alkyl, C₁-C₃ haloalkyl, C₁-C₃ alkyl-OH, C₁-C₃ alkyl-CN, or C₃-C₅ cycloalkyl optionally substituted by 1-3 R⁸ groups. In certain embodiments, X is H or —CH₃.

In certain embodiments, X is H.

In certain embodiments, X is C₁-C₆ alkyl. In certain embodiments, X is C₁-C₃ alkyl. In certain embodiments, X is methyl, ethyl, n-propyl, or isopropyl. In certain embodiments, X is —CH₃.

In certain embodiments, X is C₁-C₆ haloalkyl. In certain embodiments, X is C₁-C₆ haloalkyl containing 1-7 halogen atoms. In certain embodiments, X is C₁-C₃ haloalkyl. In certain embodiments, X is C₁-C₃ haloalkyl containing 1-7 halogen atoms. In certain embodiments, X is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, X is —CF₃, —CCl₃, —CF₂Cl, —CFCl₂, —CHF₂, —CH₂F, —CHCl₂, —CH₂Cl, or —CHFCl. In certain embodiments, X is —CF₃.

In certain embodiments, X is C₁-C₆ alkyl-OH. In certain embodiments, X is C₁-C₃ alkyl-OH. In certain embodiments, X is —CH₂OH, —CH₂CH₂—OH, —CH₂CH₂CH₂—OH, or —C(CH₃)₂—OH. In certain embodiments, X is —CH₂OH.

In certain embodiments, X is C₁-C₆ alkyl-CN. In certain embodiments, X is C₁-C₃ alkyl-CN. In certain embodiments, X is —CH₂CN, —CH₂CH₂—CN, —CH₂CH₂CH₂—CN, or —C(CH₃)₂—CN. In certain embodiments, X is —CH₂CN.

In certain embodiments, X is C₃-C₆ cycloalkyl optionally substituted by 1-5 R⁸ groups. In certain embodiments, X is C₃-C₅ cycloalkyl optionally substituted by 1-3 R⁸ groups. In certain embodiments, the cycloalkyl is substituted by 5 R⁸ groups. In certain embodiments, the cycloalkyl is substituted by 4 R⁸ groups. In certain embodiments, the cycloalkyl is substituted by 3 R⁸ groups. In certain embodiments, the cycloalkyl is substituted by 2 R⁸ groups. In certain embodiments, the cycloalkyl is substituted by 1 R⁸ group. In certain embodiments, the cycloalkyl is unsubstituted. In certain embodiments, X is cyclopropyl, cyclobutyl, or cyclopentyl, each of which is optionally substituted by 1-5 R⁸ groups. In certain embodiments, X is cyclopropyl.

In certain embodiments, X is

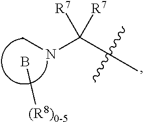

wherein the Ring B moiety, shown as

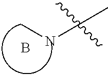

is 4- to 7-membered heterocyclyl or 5- to 8-membered heteroaryl, each of which heterocyclyl or heteroaryl optionally contains 1-2 additional heteroatoms selected from the group consisting of N, O, and S, and each of which heterocyclyl or heteroaryl is optionally substituted by 1-5 R⁸ groups. In certain embodiments, the Ring B moiety is 4- to 6-membered heterocyclyl or 5- to 6-membered heteroaryl, each of which heterocyclyl or heteroaryl optionally contains 1-2 additional heteroatoms selected from the group consisting of N, O, and S, and each of which heterocyclyl or heteroaryl is optionally substituted by 1-5 R⁸ groups. In certain embodiments, the Ring B moiety is 4- to 5-membered heterocyclyl or 5- to 6-membered heteroaryl, each of which heterocyclyl or heteroaryl optionally contains one additional heteroatom selected from the group consisting of N and O, and each of which heterocyclyl or heteroaryl is optionally substituted by 1-5 R⁸ groups.

In certain embodiments, the Ring B moiety is 4- to 7-membered heterocyclyl optionally containing 1-2 additional heteroatoms selected from the group consisting of N, O, and S, wherein the heterocyclyl is optionally substituted by 1-5 R⁸ groups. In certain embodiments, the Ring B moiety is 4- to 6-membered heterocyclyl optionally containing 1-2 additional heteroatoms selected from the group consisting of N, O, and S, wherein the heterocyclyl is optionally substituted by 1-5 R⁸ groups. In certain embodiments, the Ring B moiety is 4- to 5-membered heterocyclyl optionally containing one additional heteroatom selected from the group consisting of N and O, wherein the heterocyclyl is optionally substituted by 1-5 R⁸ groups. In certain embodiments, the heterocyclyl is substituted by 5 R⁸ groups. In certain embodiments, the heterocyclyl is substituted by 4 R⁸ groups. In certain embodiments, the heterocyclyl is substituted by 3 R⁸ groups. In certain embodiments, the heterocyclyl is substituted by 2 R⁸ groups. In certain embodiments, the heterocyclyl is substituted by one R⁸ group. In certain embodiments, the heterocyclyl is unsubstituted. In certain embodiments, the heterocyclyl contains 1-2 additional heteroatoms selected from the group consisting of nitrogen, oxygen, and sulfur. In certain embodiments, the heterocyclyl contains one additional nitrogen atom. In certain embodiments, the heterocyclyl contains two additional nitrogen atoms. In certain embodiments, the heterocyclyl further contains one oxygen atom. In certain embodiments, the heterocyclyl further contains two oxygen atoms. In certain embodiments, the heterocyclyl further contains one oxygen atom and one nitrogen atom. In certain embodiments, the heterocyclyl further contains one sulfur atom. In certain embodiments, the heterocyclyl does not contain additional heteroatoms. In certain embodiments, the heterocyclyl is azetidinyl, pyrrolidinyl, pyrazolidinyl, piperidinyl, or isoxazolidinyl, each of which is optionally substituted by 1-5 R⁸ groups.

In certain embodiments, the Ring B moiety is 5- to 8-membered heteroaryl optionally containing 1-2 additional heteroatoms selected from the group consisting of N, O, and S, wherein the heteroaryl is optionally substituted by 1-5 R⁸ groups. In certain embodiments, the Ring B moiety is 5- to 6-membered heteroaryl optionally containing 1-2 additional heteroatoms selected from the group consisting of N, O, and S, wherein the heteroaryl is optionally substituted by 1-5 R⁸ groups. In certain embodiments, the Ring B moiety is 5- to 6-membered heteroaryl optionally containing one additional heteroatom selected from the group consisting of N and O, wherein the heteroaryl is optionally substituted by 1-5 R⁸ groups. In certain embodiments, the heteroaryl is substituted by 5 R⁸ groups. In certain embodiments, the heteroaryl is substituted by 4 R⁸ groups. In certain embodiments, the heteroaryl is substituted by 3 R⁸ groups. In certain embodiments, the heteroaryl is substituted by 2 R⁸ groups. In certain embodiments, the heteroaryl is substituted by one R⁸ group. In certain embodiments, the heteroaryl is unsubstituted. In certain embodiments, the heteroaryl contains 1-2 additional heteroatoms selected from the group consisting of nitrogen, oxygen, and sulfur. In certain embodiments, the heteroaryl contains one additional nitrogen atom. In certain embodiments, the heteroaryl contains two additional nitrogen atoms. In certain embodiments, the heteroaryl further contains one oxygen atom. In certain embodiments, the heteroaryl further contains two oxygen atoms. In certain embodiments, the heteroaryl further contains one oxygen atom and one additional nitrogen atom. In certain embodiments, the heteroaryl further contains one sulfur atom. In certain embodiments, the heteroaryl does not contain additional heteroatoms. In certain embodiments, the heteroaryl is pyrrolyl, imidazolyl, pyrazolyl, isoxazolyl, oxazolyl, thiazolyl, isothiazolyl, triazolyl, pyridinyl, pyridazinyl, pyrimidinyl, pyrazinyl, or triazyl, each of which is optionally substituted by 1-5 R⁸ groups.

In certain embodiments, the Ring B moiety is

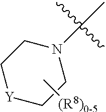

wherein Y is O, CH₂, CHR⁸, or C(R⁸)₂, and X is

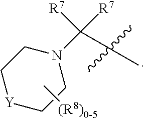

In certain embodiments, Y is oxygen (O). In other embodiments, Y is CH₂, CHR⁸, or C(R⁸)₂. In certain embodiments, Y is CH₂. In certain embodiments, Y is CHR⁸. In certain embodiments, Y is C(R⁸)₂. In certain embodiments, the Ring B moiety is substituted by a total of 1-5 R⁸ groups. In certain embodiments, the Ring B moiety is substituted by a total of 1-3 R⁸ groups. As such, if Y is CHR⁸, then the Ring B moiety can be substituted by up to 4 additional R⁸ groups. Similarly, if Y is CH(R⁸)₂, then the Ring B moiety can be substituted by up to 3 additional R⁸ groups. In certain embodiments, the Ring B moiety is substituted by 5 R⁸ groups. In certain embodiments, the Ring B moiety is substituted by 4 R⁸ groups. In certain embodiments, the Ring B moiety is substituted by 3 R⁸ groups. In certain embodiments, the Ring B moiety is substituted by 2 R⁸ groups. In certain embodiments, the Ring B moiety is substituted by one R⁸ group. In certain embodiments, the Ring B moiety is unsubstituted. In certain embodiments, the Ring B moiety is

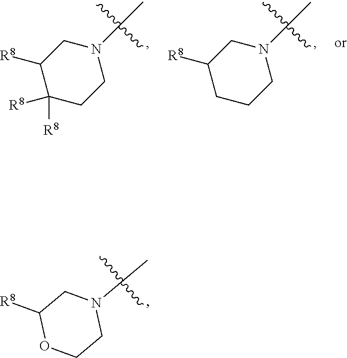

wherein each R⁸ is independently as described herein.

In certain embodiments, each R⁷ is independently H, C₁-C₆ alkyl, C₁-C₆ alkyl-OH, or C₁-C₆ haloalkyl. In certain embodiments, each R⁷ is independently H, C₁-C₃ alkyl, C₁-C₃ alkyl-OH, or C₁-C₃ haloalkyl. In certain embodiments, each R⁷ is independently H, —CH₃, —CH₂OH, or —CF₃.

In certain embodiments, both R⁷ groups are hydrogen (H). In certain embodiments, one R⁷ group is H. In certain embodiments, one R⁷ group is H, and the other R⁷ group is C₁-C₆ alkyl, C₁-C₆ alkyl-OH, or C₁-C₆ haloalkyl. In certain embodiments, one R⁷ group is H and the other R⁷ group is C₁-C₆ alkyl. In certain embodiments, one R⁷ group is H and the other R⁷ group is C₁-C₃ alkyl. In certain embodiments, one R⁷ group is H and the other R⁷ group is —CH₃.

In certain embodiments, R⁷ is C₁-C₆ alkyl. In certain embodiments, R⁷ is C₁-C₃ alkyl. In certain embodiments, R⁷ is methyl, ethyl, n-propyl, or isopropyl. In certain embodiments, one R⁷ group is methyl, ethyl, n-propyl, or isopropyl, and the other R⁷ group is H. In certain embodiments, R⁷ is —CH₃.

In certain embodiments, R⁷ is C₁-C₆ alkyl-OH. In certain embodiments, R⁷ is C₁-C₃ alkyl-OH. In certain embodiments, R⁷ is —CH₂OH, —CH₂CH₂—OH, —CH₂CH₂CH₂—OH, or —C(CH₃)₂—OH. In certain embodiments, R⁷ is —CH₂OH. In certain embodiments, one R⁷ group is C₁-C₆ alkyl-OH, and the other R⁷ group is H. In certain embodiments, one R⁷ group is C₁-C₃ alkyl-OH, and the other R⁷ group is H. In certain embodiments, one R⁷ group is —CH₂OH, —CH₂CH₂—OH, —CH₂CH₂CH₂—OH, or —C(CH₃)₂—OH, and the other R⁷ group is H. In certain embodiments, one R⁷ group is —CH₂OH, and the other R⁷ group is H.

In certain embodiments, R⁷ is C₁-C₆ haloalkyl. In certain embodiments, R⁷ is C₁-C₆ haloalkyl containing 1-7 halogen atoms. In certain embodiments, R⁷ is C₁-C₃ haloalkyl. In certain embodiments, R⁷ is C₁-C₃ haloalkyl containing 1-7 halogen atoms. In certain embodiments, R⁷ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁷ is —CF₃, —CCl₃, —CF₂Cl, —CFCl₂, —CHF₂, —CH₂F, —CHCl₂, —CH₂F, or —CHFCl. In certain embodiments, R⁷ is —CF₃.

In certain embodiments, two R⁷ groups are taken together with the carbon atom to which they are attached to form C₃-C₅ cycloalkyl or 3- to 5-membered heterocyclyl. In certain embodiments, two R⁷ groups are taken together with the carbon atom to which they are attached to form cyclopropyl or oxetanyl.

In certain embodiments, two R⁷ groups are taken together with the carbon atom to which they are attached to form C₃-C₅ cycloalkyl. In certain embodiments, two R⁷ groups are taken together with the carbon atom to which they are attached to form cyclopropyl or cyclobutyl. In certain embodiments, two R⁷ groups are taken together with the carbon atom to which they are attached to form cyclopropyl.

In certain embodiments, two R⁷ groups are taken together with the carbon atom to which they are attached to form 3- to 5-membered heterocyclyl. In certain embodiments, two R⁷ groups are taken together with the carbon atom to which they are attached to form 3- to 4-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁷ is aziridinyl, oxiranyl, oxetanyl, azetidinyl, tetrahydrofuranyl, dioxolanyl, pyrrolidinyl, pyrazolidinyl, or isoxazolidinyl.

In certain embodiments, each R⁸ is independently halo, C₁-C₆ alkyl, C₁-C₆ alkyl-CN, C₁-C₆ alkyl-OH, C₁-C₆ haloalkyl, —CN, oxo, or —O(C₁-C₆ alkyl). In certain embodiments, each R⁸ is independently halo, C₁-C₃ alkyl, C₁-C₃ alkyl-CN, C₁-C₃ alkyl-OH, C₁-C₃ haloalkyl, —CN, oxo, or —O(C₁-C₃ alkyl). In certain embodiments, each R⁸ is independently fluoro (F), —CH₃, —CH₂CH₃, —CH₂CN, —CH₂OH, —CF₃, —CN, oxo, or —OCH₃.

In certain embodiments, R⁸ is halo. In certain embodiments, R⁸ is chloro, fluoro, or bromo. In certain embodiments, R⁸ is chloro or fluoro. In certain embodiments, R⁸ is fluoro.

In certain embodiments, R⁸ is C₁-C₆ alkyl. In certain embodiments, R⁸ is C₁-C₃ alkyl. In certain embodiments, R⁸ is methyl, ethyl, n-propyl, or isopropyl. In certain embodiments, R⁸ is —CH₃ or —CH₂CH₃.

In certain embodiments, R⁸ is —CN. In certain embodiments, R⁸ is C₁-C₆ alkyl-CN. In certain embodiments, R⁸ is C₁-C₃ alkyl-CN. In certain embodiments, R⁸ is —CH₂CN, —CH₂CH₂—CN, —CH₂CH₂CH₂—CN, or —C(CH₃)₂—CN. In certain embodiments, R⁸ is —CH₂CN.

In certain embodiments, R⁸ is C₁-C₆ alkyl-OH. In certain embodiments, R⁸ is C₁-C₃ alkyl-OH. In certain embodiments, R⁸ is —CH₂OH, —CH₂CH₂—OH, —CH₂CH₂CH₂—OH, or —C(CH₃)₂—OH. In certain embodiments, R⁸ is —CH₂OH.

In certain embodiments, R⁸ is C₁-C₆ haloalkyl. In certain embodiments, R⁸ is C₁-C₆ haloalkyl containing 1-7 halogen atoms. In certain embodiments, R⁸ is C₁-C₃ haloalkyl. In certain embodiments, R⁸ is C₁-C₃ haloalkyl containing 1-7 halogen atoms. In certain embodiments, R⁸ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁸ is —CF₃, —CCl₃, —CF₂Cl, —CFCl₂, —CHF₂, —CH₂F, —CHCl₂, —CH₂F, or —CHFCl. In certain embodiments, R⁸ is —CF₃.

In certain embodiments, R⁸ is oxo.

In certain embodiments, R⁸ is —O(C₁-C₆ alkyl). In certain embodiments, R⁸ is —O—(C₁-C₃ alkyl). In certain embodiments, R⁸ is —O(methyl), —O(ethyl), —O(n-propyl), or —O(isopropyl). In certain embodiments, R⁸ is —OCH₃ or —OCH₂CH₃. In certain embodiments, R⁸ is —OCH₃.

In certain embodiments, two R⁸ groups are taken together with the carbon atom or atoms to which they are attached to form a spiro or fused C₃-C₅ cycloalkyl or 3- to 5-membered heterocyclyl. In certain embodiments, two R⁸ groups are taken together with the carbon atom or atoms to which they are attached to form a spiro or fused cyclopropyl or oxetanyl.

In certain embodiments, two R⁸ groups are taken together with the carbon atom or atoms to which they are attached to form a spiro or fused C₃-C₅ cycloalkyl. In certain embodiments, two R⁸ groups are taken together with the carbon atom or atoms to which they are attached to form a spiro C₃-C₅ cycloalkyl. In certain embodiments, two R⁸ groups are taken together with the carbon atom or atoms to which they are attached to form a spiro cyclopropyl. In certain embodiments, two R⁸ groups are taken together with the carbon atom or atoms to which they are attached to form a spiro cyclobutyl. In certain embodiments, two R⁸ groups are taken together with the carbon atom or atoms to which they are attached to form a spiro cyclopentyl. In certain embodiments, two R⁸ groups are taken together with the carbon atom or atoms to which they are attached to form a fused C₃-C₅ cycloalkyl. In certain embodiments, two R⁸ groups are taken together with the carbon atom or atoms to which they are attached to form a fused cyclopropyl. In certain embodiments, two R⁸ groups are taken together with the carbon atom or atoms to which they are attached to form a fused cyclobutyl. In certain embodiments, two R⁸ groups are taken together with the carbon atom or atoms to which they are attached to form a fused cyclopentyl. In certain embodiments, two R⁸ groups are taken together with the carbon atom or atoms to which they are attached to form a spiro or fused cyclopropyl.

In certain embodiments, two R⁸ groups are taken together with the carbon atom or atoms to which they are attached to form a spiro or fused 3- to 5-membered heterocyclyl. In certain embodiments, two R⁸ groups are taken together with the carbon atom or atoms to which they are attached to form a spiro 3- to 5-membered heterocyclyl. In certain embodiments, two R⁸ groups are taken together with the carbon atom or atoms to which they are attached to form a spiro oxetanyl. In certain embodiments, two R⁸ groups are taken together with the carbon atom or atoms to which they are attached to form a fused 3- to 5-membered heterocyclyl. In certain embodiments, two R⁸ groups are taken together with the carbon atom or atoms to which they are attached to form a fused oxetanyl. In certain embodiments, two R⁸ groups are taken together with the carbon atom or atoms to which they are attached to form a spiro or fused oxetanyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁸ groups are taken together with the carbon atom to which they are attached to form a spiro aziridinyl, oxiranyl, oxetanyl, azetidinyl, tetrahydrofuranyl, dioxolanyl, pyrrolidinyl, pyrazolidinyl, or isoxazolidinyl. In certain embodiments, two R⁸ groups are taken together with the carbon atoms to which they are attached to form a fused aziridinyl, oxiranyl, oxetanyl, azetidinyl, tetrahydrofuranyl, dioxolanyl, pyrrolidinyl, pyrazolidinyl, or isoxazolidinyl.

In certain embodiments, X is

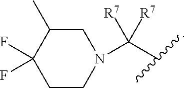

In certain embodiments, X is

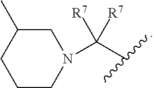

In certain embodiments, X is

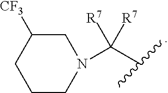

In certain embodiments, X is

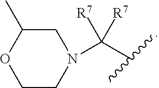

In certain embodiments, X is

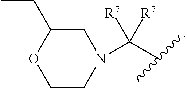

In certain embodiments, X is

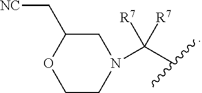

In any of these embodiments, both R⁷ groups can be hydrogen (H). In any of these embodiments, one R⁷ group can be H and one R⁷ group can be —CH₃. In any of these embodiments, both R⁷ groups can be —CH₃.

In certain embodiments, the compound is of Formula (I-A) or (I-B):

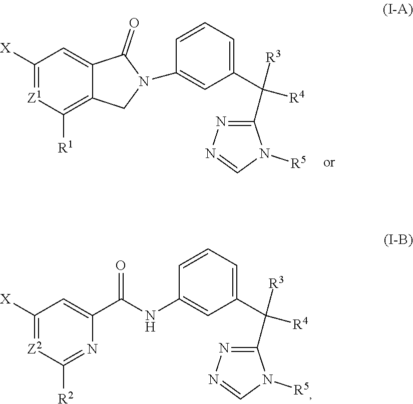

wherein R′, R², R³, R⁴, R⁵, Z¹, Z², and X are as described for the compound of Formula (I).

In certain embodiments, the compound is of Formula (I-a) or (I-b):

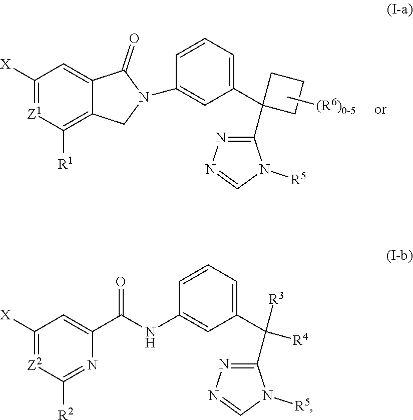

wherein R′, R², R⁵, R⁶, Z¹, Z², and X are as described for the compound of Formula (I).

In certain embodiments, the compound is of Formula (I-C), (I-D), (I-E), (I-F), (I-G), (I-H), or (I-J):

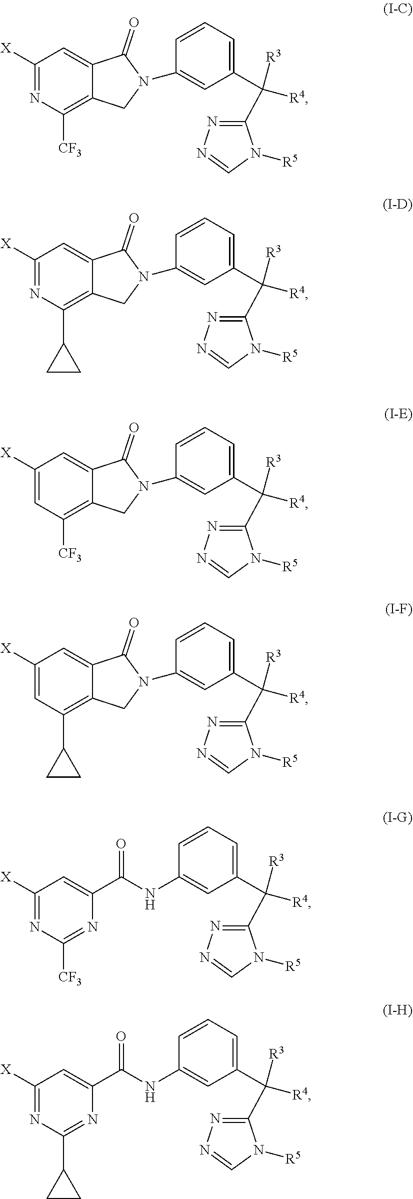

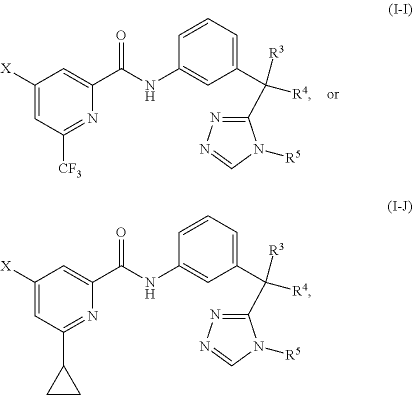

wherein R³, R⁴, R⁵, and X are as described for the compound of Formula (I).

In certain embodiments, the compound is of Formula (II-A), (II-B), (II-C), (II-D), (II-E), (II-F), (II-G), or (II-H):

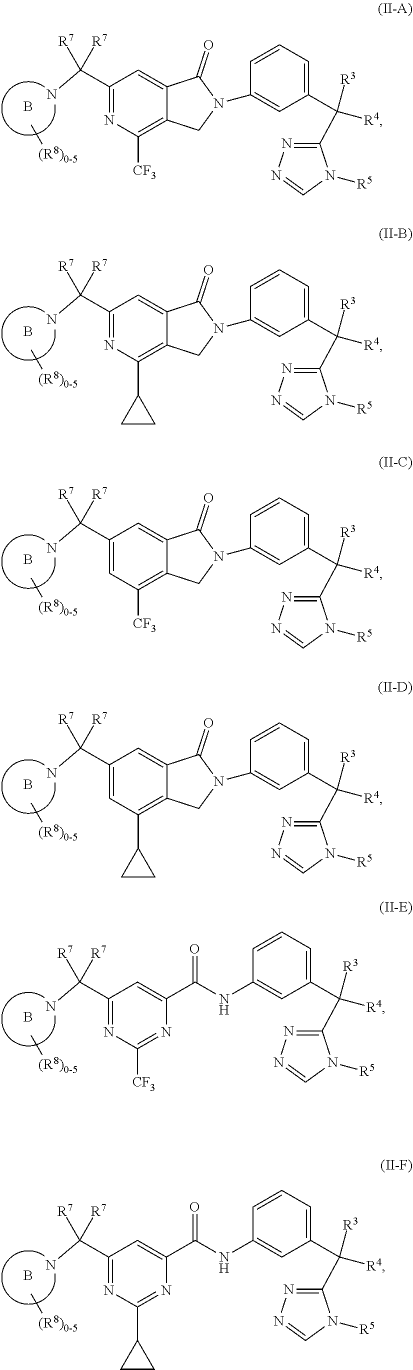

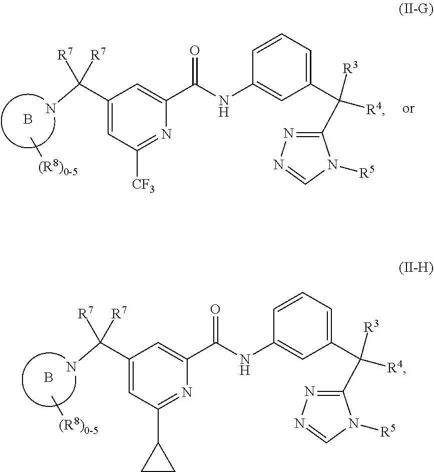

wherein R³, R⁴, R⁵, R⁷, R⁸ and the Ring B moiety are as described for the compound of Formula (I).

In certain embodiments, the compound is of (III-A), (III-B), (III-C), (III-D), (III-E), (III-F), (III-G), or (III-H):

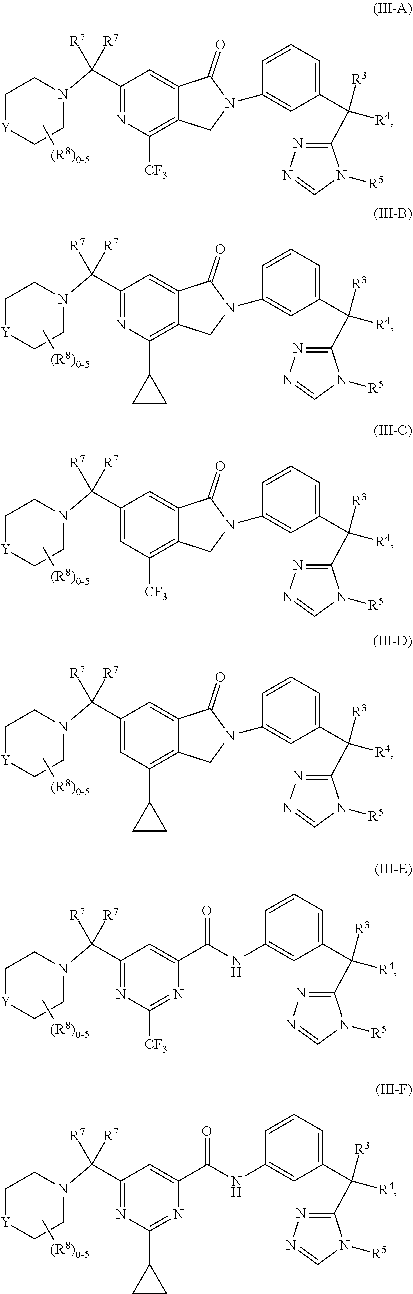

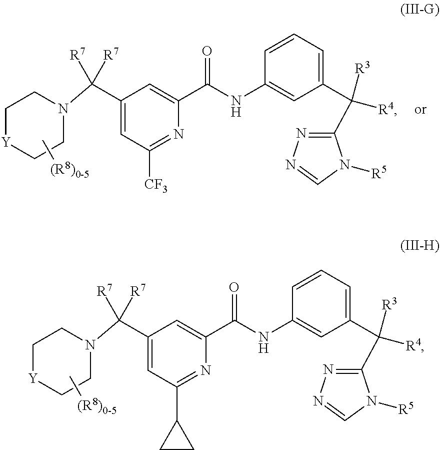

wherein R³, R⁴, R⁵, R⁷, R⁸, and Y are as described for the compound of Formula (I).

In certain embodiments, the compound is of (IV-A), (IV-B), (IV-C), (IV-D), (IV-E), (IV-F), (IV-G), or (IV-H):

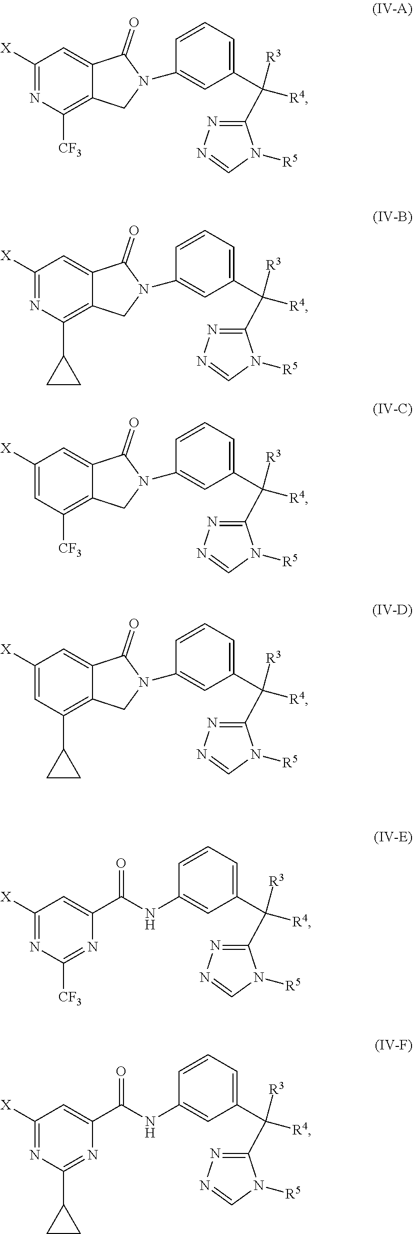

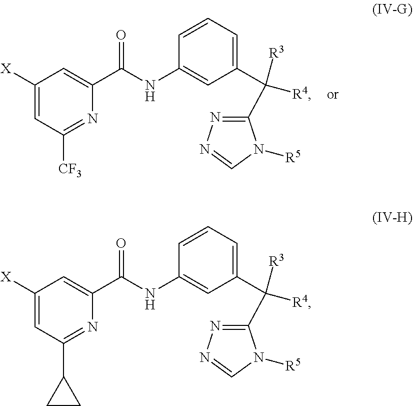

wherein R³, R⁴, and R⁵ are as described for the compound of Formula (I), and X is H, C₁-C₆ alkyl, C₁-C₆ haloalkyl, C₁-C₆ alkyl-OH, C₁-C₆ alkyl-CN, or C₃-C₆ cycloalkyl optionally substituted by 1-5 R⁸ groups. In certain embodiments, X is H. In certain embodiments, X is C₁-C₆ alkyl. In certain embodiments, X is C₁-C₆ haloalkyl. In certain embodiments, X is C₁-C₆ alkyl-OH. In certain embodiments, X is C₁-C₆ alkyl-CN. In certain embodiments, X is C₃-C₆ cycloalkyl optionally substituted by 1-5 R⁸ groups.

In certain embodiments, the compound is from Table 1, or a pharmaceutically acceptable stereoisomer, tautomer, or salt thereof.

TABLE 1
Representative Compounds of This Disclosure

| Cmpd | |
|---|---|
| No. | Structure |
| 1 | 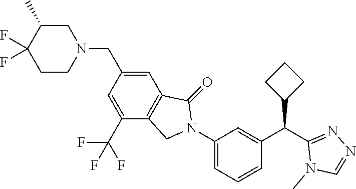 [SMILES: C[C@@H]1CN(Cc2cc3c(c(C(F)(F)F)c2)CN(c2cccc([C@H](c4nncn4C)C4CCC4)c2)C3=O)CCC1(F)F] |
| 2 | 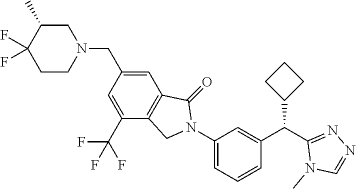 [SMILES: C[C@@H]1CN(Cc2cc3c(c(C(F)(F)F)c2)CN(c2cccc([C@@H](c4nncn4C)C4CCC4)c2)C3=O)CCC1(F)F] |
| 3 | 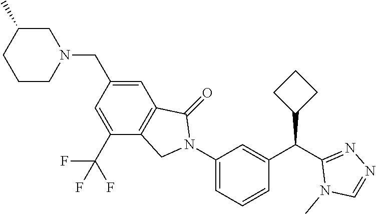 [SMILES: C[C@H]1CCCN(Cc2cc3c(c(C(F)(F)F)c2)CN(c2cccc([C@H](c4nncn4C)C4CCC4)c2)C3=O)C1] |
| 4 | 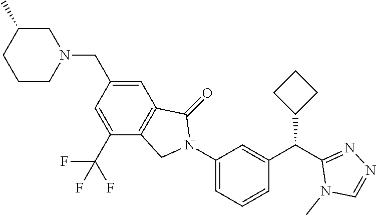 [SMILES: C[C@H]1CCCN(Cc2cc3c(c(C(F)(F)F)c2)CN(c2cccc([C@@H](c4nncn4C)C4CCC4)c2)C3=O)C1] |
| 5 | 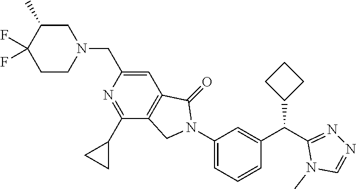 [SMILES: C[C@@H]1CN(Cc2cc3c(c(C4CC4)n2)CN(c2cccc([C@@H](c4nncn4C)C4CCC4)c2)C3=O)CCC1(F)F] |
| 6 | 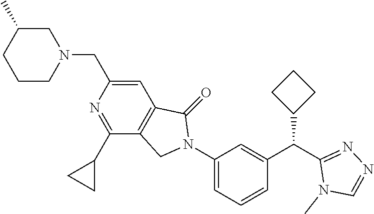 [SMILES: C[C@H]1CCCN(Cc2cc3c(c(C4CC4)n2)CN(c2cccc([C@@H](c4nncn4C)C4CCC4)c2)C3=O)C1] |
| 7 | 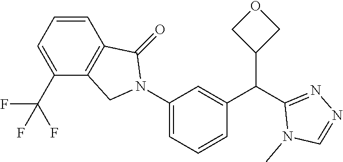 [SMILES: Cn1cnnc1C(c1cccc(N2Cc3c(cccc3C(F)(F)F)C2=O)c1)C1COC1] |
| 8 | 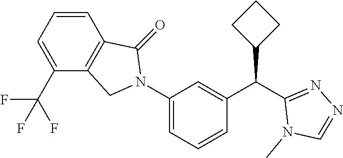 [SMILES: Cn1cnnc1[C@@H](c1cccc(N2Cc3c(cccc3C(F)(F)F)C2=O)c1)C1CCC1] |
| 9 | 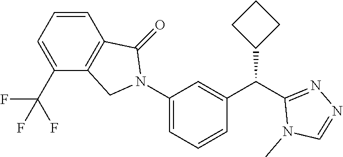 [SMILES: Cn1cnnc1[C@H](c1cccc(N2Cc3c(cccc3C(F)(F)F)C2=O)c1)C1CCC1] |
| 10 | 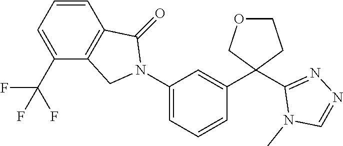 [SMILES: Cn1cnnc1C1(c2cccc(N3Cc4c(cccc4C(F)(F)F)C3=O)c2)CCOC1] |
| 11 | 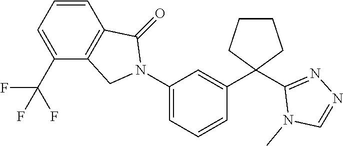 [SMILES: Cn1cnnc1C1(c2cccc(N3Cc4c(cccc4C(F)(F)F)C3=O)c2)CCCC1] |
| 12 | 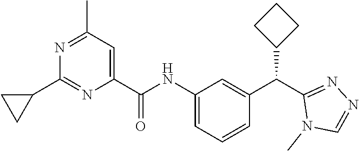 [SMILES: Cc1cc(C(=O)Nc2cccc([C@@H](c3nncn3C)C3CCC3)c2)nc(C2CC2)n1] |
| 13 | 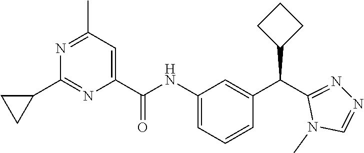 [SMILES: Cc1cc(C(=O)Nc2cccc([C@H](c3nncn3C)C3CCC3)c2)nc(C2CC2)n1] |
| 14 | 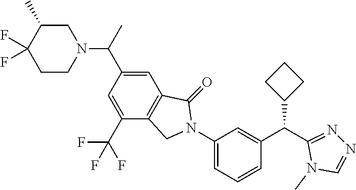 [SMILES: CC(c1cc2c(c(C(F)(F)F)c1)CN(c1cccc([C@@H](c3nncn3C)C3CCC3)c1)C2=O)N1CCC(F)(F)[C@H](C)C1] |
| 15 | 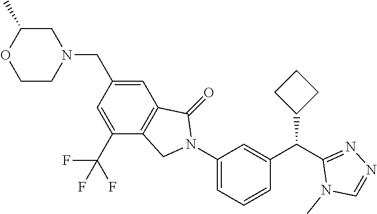 [SMILES: C[C@@H]1CN(Cc2cc3c(c(C(F)(F)F)c2)CN(c2cccc([C@@H](c4nncn4C)C4CCC4)c2)C3=O)CCO1] |
| 16 | 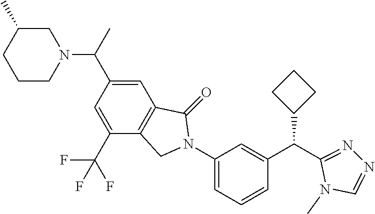 [SMILES: CC(c1cc2c(c(C(F)(F)F)c1)CN(c1cccc([C@@H](c3nncn3C)C3CCC3)c1)C2=O)N1CCC[C@H](C)C1] |
| 17 | 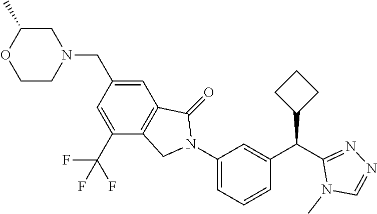 [SMILES: C[C@@H]1CN(Cc2cc3c(c(C(F)(F)F)c2)CN(c2cccc([C@H](c4nncn4C)C4CCC4)c2)C3=O)CCO1] |
| 18 | 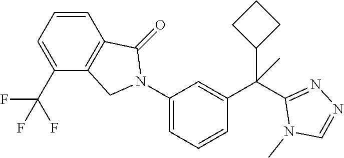 [SMILES: Cn1cnnc1C(C)(c1cccc(N2Cc3c(cccc3C(F)(F)F)C2=O)c1)C1CCC1] |
| 19 | 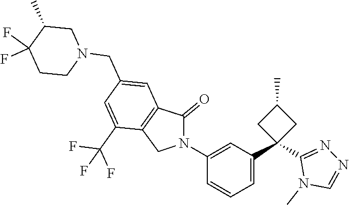 [SMILES: C[C@@H]1CN(Cc2cc3c(c(C(F)(F)F)c2)CN(c2cccc([C@]4(c5nncn5C)C[C@@H](C)C4)c2)C3=O)CCC1(F)F] |
| 20 | 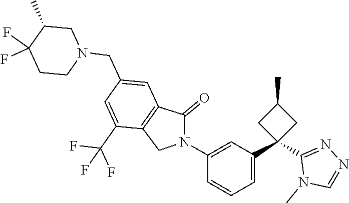 [SMILES: C[C@@H]1CN(Cc2cc3c(c(C(F)(F)F)c2)CN(c2cccc([C@]4(c5nncn5C)C[C@H](C)C4)c2)C3=O)CCC1(F)F] |
| 21 | 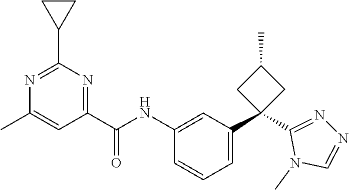 [SMILES: Cc1cc(C(=O)Nc2cccc([C@]3(c4nncn4C)C[C@@H](C)C3)c2)nc(C2CC2)n1] |
| 22 | 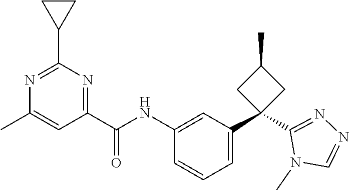 [SMILES: Cc1cc(C(=O)Nc2cccc([C@]3(c4nncn4C)C[C@H](C)C3)c2)nc(C2CC2)n1] |
| 23 | 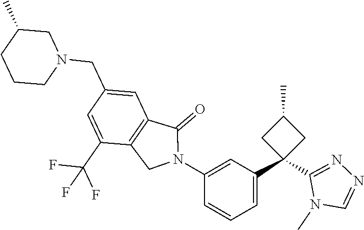 [SMILES: C[C@H]1CCCN(Cc2cc3c(c(C(F)(F)F)c2)CN(c2cccc([C@]4(c5nncn5C)C[C@@H](C)C4)c2)C3=O)C1] |
| 24 | 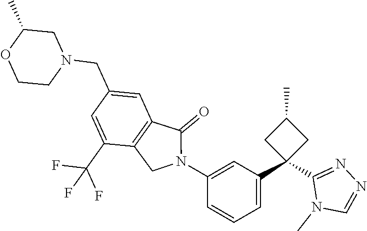 [SMILES: C[C@@H]1CN(Cc2cc3c(c(C(F)(F)F)c2)CN(c2cccc([C@]4(c5nncn5C)C[C@@H](C)C4)c2)C3=O)CCO1] |
| 25 | 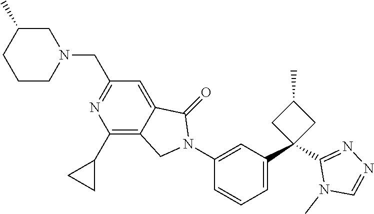 [SMILES: C[C@H]1CCCN(Cc2cc3c(c(C4CC4)n2)CN(c2cccc([C@]4(c5nncn5C)C[C@@H](C)C4)c2)C3=O)C1] |
| 26 | 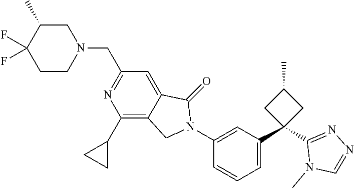 [SMILES: C[C@@H]1CN(Cc2cc3c(c(C4CC4)n2)CN(c2cccc([C@]4(c5nncn5C)C[C@@H](C)C4)c2)C3=O)CCC1(F)F] |
| 27 | 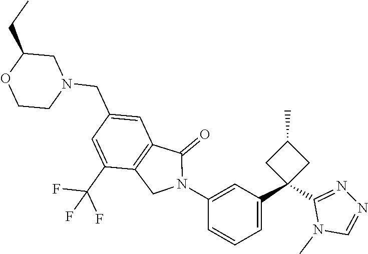 [SMILES: CC[C@H]1CN(Cc2cc3c(c(C(F)(F)F)c2)CN(c2cccc([C@]4(c5nncn5C)C[C@@H](C)C4)c2)C3=O)CCO1] |
| 28 | 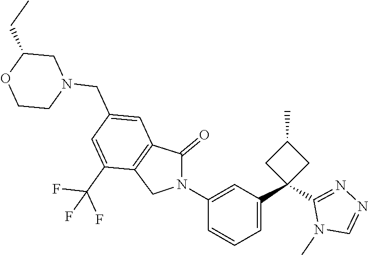 [SMILES: CC[C@@H]1CN(Cc2cc3c(c(C(F)(F)F)c2)CN(c2cccc([C@]4(c5nncn5C)C[C@@H](C)C4)c2)C3=O)CCO1] |
| 29 | 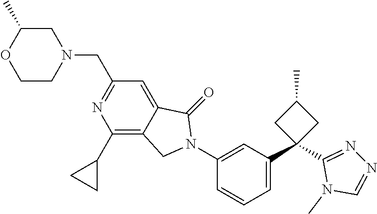 [SMILES: C[C@@H]1CN(Cc2cc3c(c(C4CC4)n2)CN(c2cccc([C@]4(c5nncn5C)C[C@@H](C)C4)c2)C3=O)CCO1] |
| 30 | 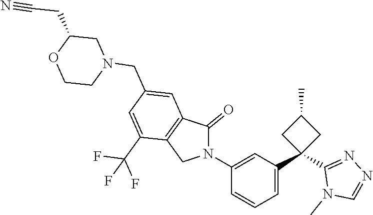 [SMILES: Cn1cnnc1[C@]1(c2cccc(N3Cc4c(cc(CN5CCO[C@H](CC#N)C5)cc4C(F)(F)F)C3=O)c2)C[C@H](C)C1] |
| 31 | 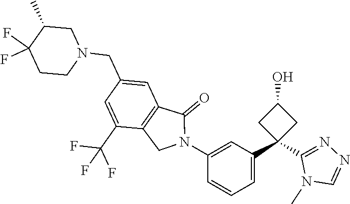 [SMILES: C[C@@H]1CN(Cc2cc3c(c(C(F)(F)F)c2)CN(c2cccc([C@]4(c5nncn5C)C[C@@H](O)C4)c2)C3=O)CCC1(F)F] |
| 32 | 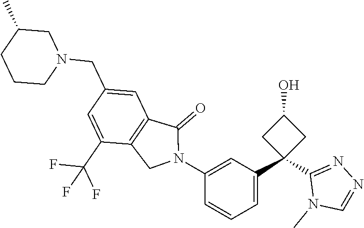 [SMILES: C[C@H]1CCCN(Cc2cc3c(c(C(F)(F)F)c2)CN(c2cccc([C@]4(c5nncn5C)C[C@@H](O)C4)c2)C3=O)C1] |
| 33 | 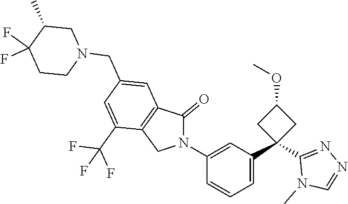 [SMILES: CO[C@H]1C[C@@](c2cccc(N3Cc4c(cc(CN5CCC(F)(F)[C@H](C)C5)cc4C(F)(F)F)C3=O)c2)(c2nncn2C)C1] |
| 34 | 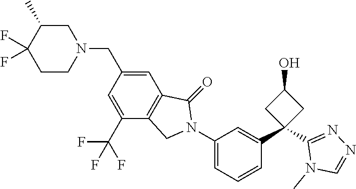 [SMILES: C[C@@H]1CN(Cc2cc3c(c(C(F)(F)F)c2)CN(c2cccc([C@]4(c5nncn5C)C[C@H](O)C4)c2)C3=O)CCC1(F)F] |
| 35 | 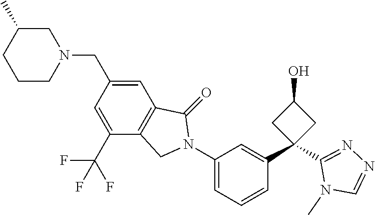 [SMILES: C[C@H]1CCCN(Cc2cc3c(c(C(F)(F)F)c2)CN(c2cccc([C@]4(c5nncn5C)C[C@H](O)C4)c2)C3=O)C1] |
| 36 | 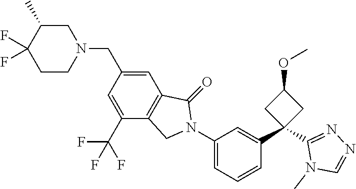 [SMILES: CO[C@H]1C[C@](c2cccc(N3Cc4c(cc(CN5CCC(F)(F)[C@H](C)C5)cc4C(F)(F)F)C3=O)c2)(c2nncn2C)C1] |
| 37 | 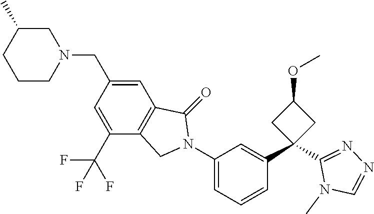 [SMILES: CO[C@H]1C[C@](c2cccc(N3Cc4c(cc(CN5CCC[C@H](C)C5)cc4C(F)(F)F)C3=O)c2)(c2nncn2C)C1] |
| 38 | 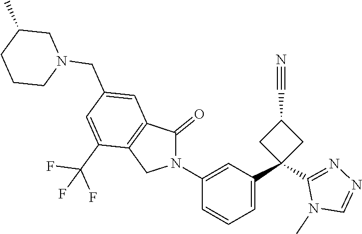 [SMILES: C[C@H]1CCCN(Cc2cc3c(c(C(F)(F)F)c2)CN(c2cccc([C@]4(c5nncn5C)C[C@@H](C#N)C4)c2)C3=O)C1] |
| 39 | 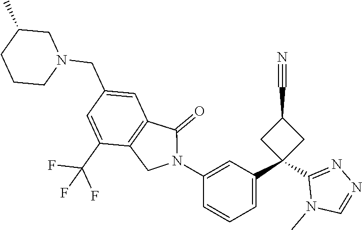 [SMILES: C[C@H]1CCCN(Cc2cc3c(c(C(F)(F)F)c2)CN(c2cccc([C@]4(c5nncn5C)C[C@H](C#N)C4)c2)C3=O)C1] |
| 40 | 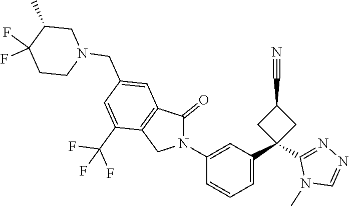 [SMILES: C[C@@H]1CN(Cc2cc3c(c(C(F)(F)F)c2)CN(c2cccc([C@]4(c5nncn5C)C[C@H](C#N)C4)c2)C3=O)CCC1(F)F] |
| 41 | 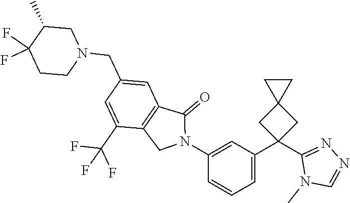 [SMILES: C[C@@H]1CN(Cc2cc3c(c(C(F)(F)F)c2)CN(c2cccc(C4(c5nncn5C)CC5(CC5)C4)c2)C3=O)CCC1(F)F] |
| 42 | 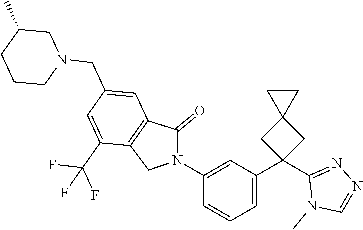 [SMILES: C[C@H]1CCCN(Cc2cc3c(c(C(F)(F)F)c2)CN(c2cccc(C4(c5nncn5C)CC5(CC5)C4)c2)C3=O)C1] |
| 43 | 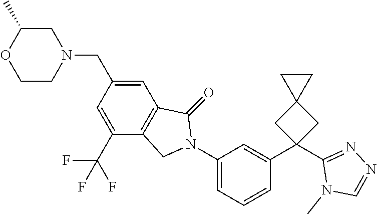 [SMILES: C[C@@H]1CN(Cc2cc3c(c(C(F)(F)F)c2)CN(c2cccc(C4(c5nncn5C)CC5(CC5)C4)c2)C3=O)CCO1] |
| 44 | 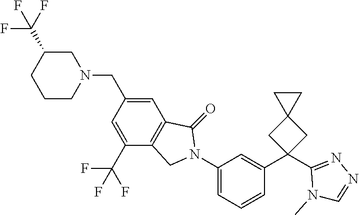 [SMILES: Cn1cnnc1C1(c2cccc(N3Cc4c(cc(CN5CCC[C@H](C(F)(F)F)C5)cc4C(F)(F)F)C3=O)c2)CC2(CC2)C1] |
| 45 | 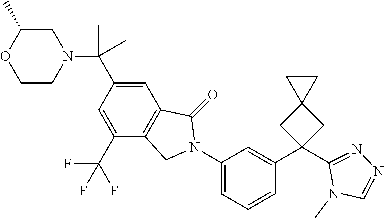 [SMILES: C[C@@H]1CN(C(C)(C)c2cc3c(c(C(F)(F)F)c2)CN(c2cccc(C4(c5nncn5C)CC5(CC5)C4)c2)C3=O)CCO1] |
| 46 | 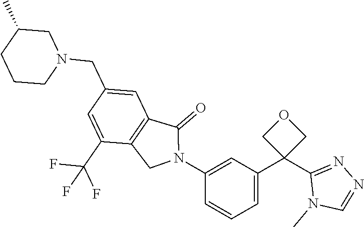 [SMILES: C[C@H]1CCCN(Cc2cc3c(c(C(F)(F)F)c2)CN(c2cccc(C4(c5nncn5C)COC4)c2)C3=O)C1] |
| 47 | 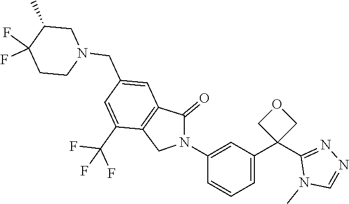 [SMILES: C[C@@H]1CN(Cc2cc3c(c(C(F)(F)F)c2)CN(c2cccc(C4(c5nncn5C)COC4)c2)C3=O)CCC1(F)F] |
| 48 | 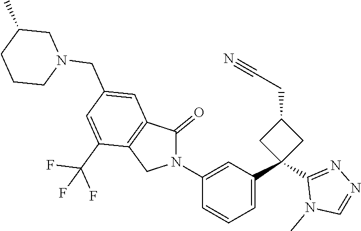 [SMILES: C[C@H]1CCCN(Cc2cc3c(c(C(F)(F)F)c2)CN(c2cccc([C@]4(c5nncn5C)C[C@@H](CC#N)C4)c2)C3=O)C1] |
| 49 | 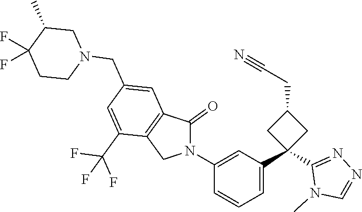 [SMILES: C[C@@H]1CN(Cc2cc3c(c(C(F)(F)F)c2)CN(c2cccc([C@]4(c5nncn5C)C[C@@H](CC#N)C4)c2)C3=O)CCC1(F)F] |
| 50 | 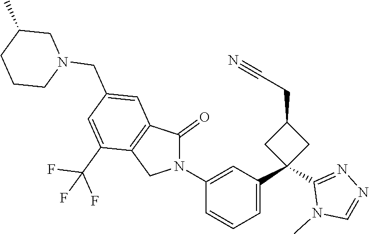 [SMILES: C[C@H]1CCCN(Cc2cc3c(c(C(F)(F)F)c2)CN(c2cccc([C@]4(c5nncn5C)C[C@H](CC#N)C4)c2)C3=O)C1] |
| 51 | 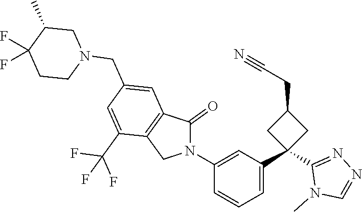 [SMILES: C[C@@H]1CN(Cc2cc3c(c(C(F)(F)F)c2)CN(c2cccc([C@]4(c5nncn5C)C[C@H](CC#N)C4)c2)C3=O)CCC1(F)F] |
| 52 | 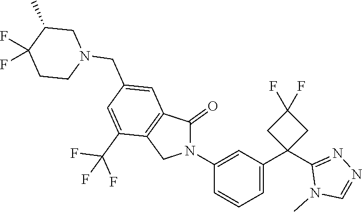 [SMILES: C[C@@H]1CN(Cc2cc3c(c(C(F)(F)F)c2)CN(c2cccc(C4(c5nncn5C)CC(F)(F)C4)c2)C3=O)CCC1(F)F] |
| 53 | 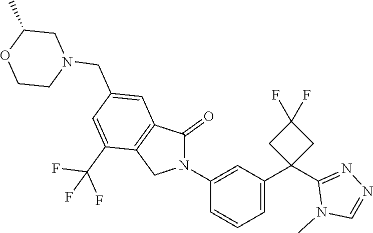 [SMILES: C[C@@H]1CN(Cc2cc3c(c(C(F)(F)F)c2)CN(c2cccc(C4(c5nncn5C)CC(F)(F)C4)c2)C3=O)CCO1] |

In certain embodiments, the compound is from Table 2, or a pharmaceutically acceptable stereoisomer, tautomer, or salt thereof.

TABLE 2
Representative Compounds of This Disclosure

| Cmpd No. | Structure |
|---|---|
| 101 | 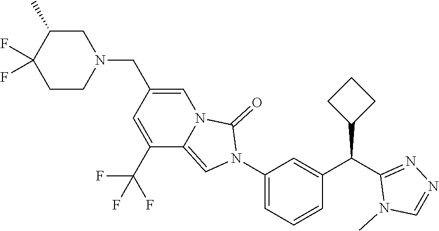 [SMILES: C[C@@H]1CN(Cc2cc(C(F)(F)F)c3cn(-c4cccc([C@H](c5nncn5C)C5CCC5)c4)c(=O)n3c2)CCC1(F)F] |
| 102 | 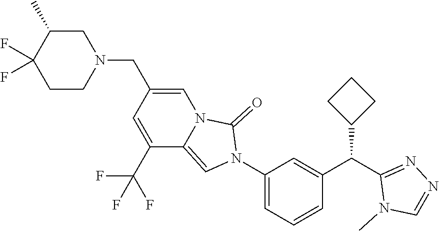 [SMILES: C[C@@H]1CN(Cc2cc(C(F)(F)F)c3cn(-c4cccc([C@@H](c5nncn5C)C5CCC5)c4)c(=O)n3c2)CCC1(F)F] |
| 103 | 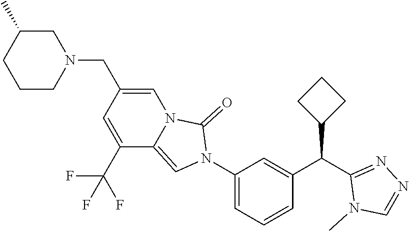 [SMILES: C[C@H]1CCCN(Cc2cc(C(F)(F)F)c3cn(-c4cccc([C@H](c5nncn5C)C5CCC5)c4)c(=O)n3c2)C1] |
| 104 | 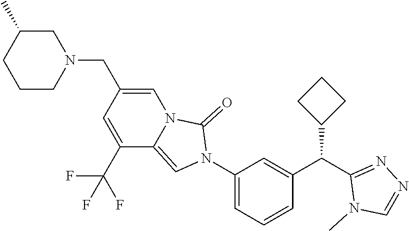 [SMILES: C[C@H]1CCCN(Cc2cc(C(F)(F)F)c3cn(-c4cccc([C@@H](c5nncn5C)C5CCC5)c4)c(=O)n3c2)C1] |
| 105 | 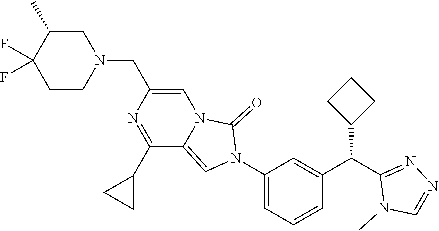 [SMILES: C[C@@H]1CN(Cc2cn3c(=O)n(-c4cccc([C@@H](c5nncn5C)C5CCC5)c4)cc3c(C3CC3)n2)CCC1(F)F] |
| 106 | 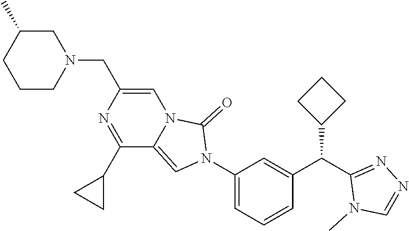 [SMILES: C[C@H]1CCCN(Cc2cn3c(=O)n(-c4cccc([C@@H](c5nncn5C)C5CCC5)c4)cc3c(C3CC3)n2)C1] |
| 107 | 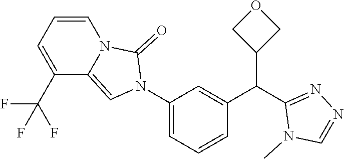 [SMILES: Cn1cnnc1C(c1cccc(-n2cc3c(C(F)(F)F)cccn3c2=O)c1)C1COC1] |
| 108 | 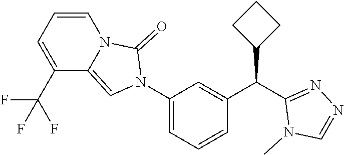 [SMILES: Cn1cnnc1[C@@H](c1cccc(-n2cc3c(C(F)(F)F)cccn3c2=O)c1)C1CCC1] |
| 109 | 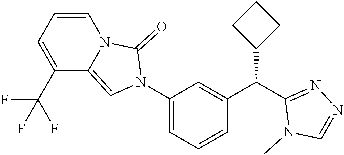 [SMILES: Cn1cnnc1[C@H](c1cccc(-n2cc3c(C(F)(F)F)cccn3c2=O)c1)C1CCC1] |
| 110 | 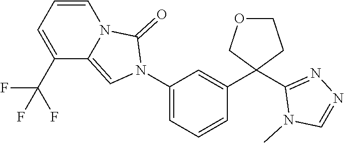 [SMILES: Cn1cnnc1C1(c2cccc(-n3cc4c(C(F)(F)F)cccn4c3=O)c2)CCOC1] |
| 111 | 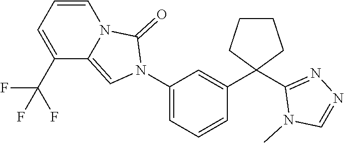 [SMILES: Cn1cnnc1C1(c2cccc(-n3cc4c(C(F)(F)F)cccn4c3=O)c2)CCCC1] |
| 114 | 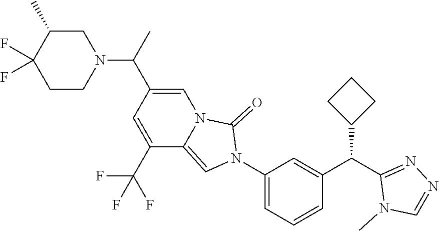 [SMILES: CC(c1cc(C(F)(F)F)c2cn(-c3cccc([C@@H](c4nncn4C)C4CCC4)c3)c(=O)n2c1)N1CCC(F)(F)[C@H](C)C1] |
| 115 | 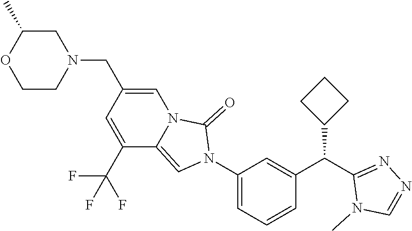 [SMILES: C[C@@H]1CN(Cc2cc(C(F)(F)F)c3cn(-c4cccc([C@@H](c5nncn5C)C5CCC5)c4)c(=O)n3c2)CCO1] |
| 116 | 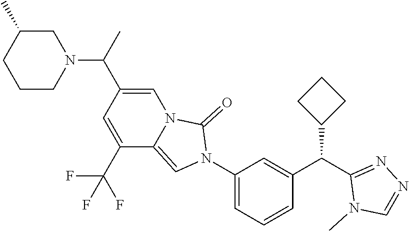 [SMILES: CC(c1cc(C(F)(F)F)c2cn(-c3cccc([C@@H](c4nncn4C)C4CCC4)c3)c(=O)n2c1)N1CCC[C@H](C)C1] |
| 117 | 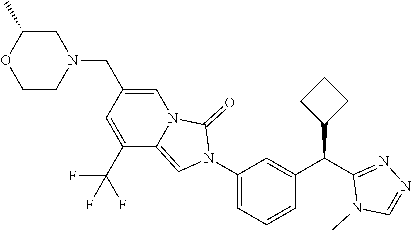 [SMILES: C[C@@H]1CN(Cc2cc(C(F)(F)F)c3cn(-c4cccc([C@H](c5nncn5C)C5CCC5)c4)c(=O)n3c2)CCO1] |
| 118 | 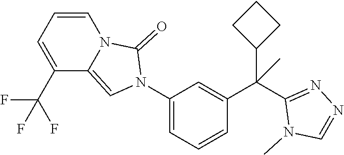 [SMILES: Cn1cnnc1C(C)(c1cccc(-n2cc3c(C(F)(F)F)cccn3c2=O)c1)C1CCC1] |
| 119 | 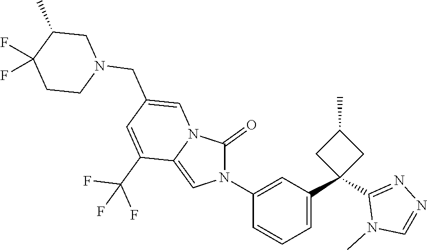 [SMILES: C[C@@H]1CN(Cc2cc(C(F)(F)F)c3cn(-c4cccc([C@]5(c6nncn6C)C[C@@H](C)C5)c4)c(=O)n3c2)CCC1(F)F] |
| 120 | 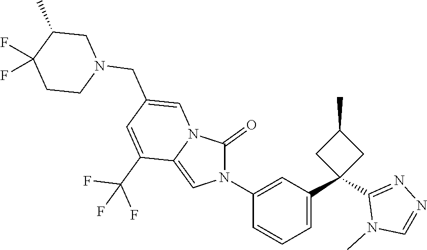 [SMILES: C[C@@H]1CN(Cc2cc(C(F)(F)F)c3cn(-c4cccc([C@]5(c6nncn6C)C[C@H](C)C5)c4)c(=O)n3c2)CCC1(F)F] |
| 123 | 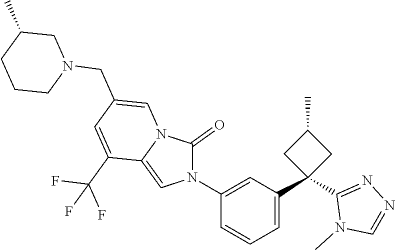 [SMILES: C[C@H]1CCCN(Cc2cc(C(F)(F)F)c3cn(-c4cccc([C@]5(c6nncn6C)C[C@@H](C)C5)c4)c(=O)n3c2)C1] |
| 124 | 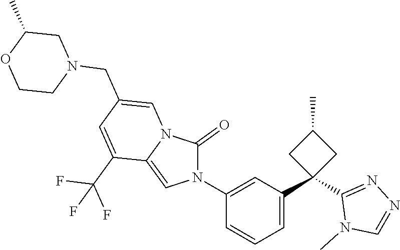 [SMILES: C[C@@H]1CN(Cc2cc(C(F)(F)F)c3cn(-c4cccc([C@]5(c6nncn6C)C[C@@H](C)C5)c4)c(=O)n3c2)CCO1] |
| 125 | 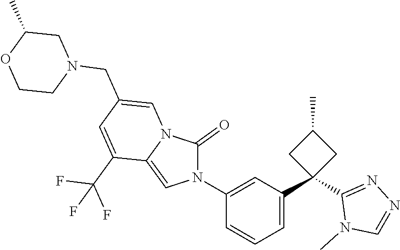 [SMILES: C[C@@H]1CN(Cc2cc(C(F)(F)F)c3cn(-c4cccc([C@]5(c6nncn6C)C[C@@H](C)C5)c4)c(=O)n3c2)CCO1] |
| 126 | 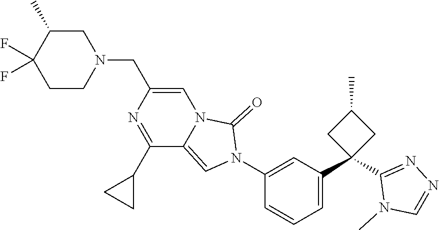 [SMILES: C[C@@H]1CN(Cc2cn3c(=O)n(-c4cccc([C@]5(c6nncn6C)C[C@@H](C)C5)c4)cc3c(C3CC3)n2)CCC1(F)F] |
| 127 | 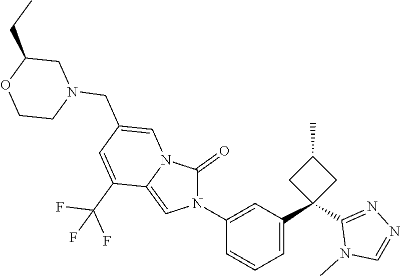 [SMILES: CC[C@H]1CN(Cc2cc(C(F)(F)F)c3cn(-c4cccc([C@]5(c6nncn6C)C[C@@H](C)C5)c4)c(=O)n3c2)CCO1] |
| 128 | 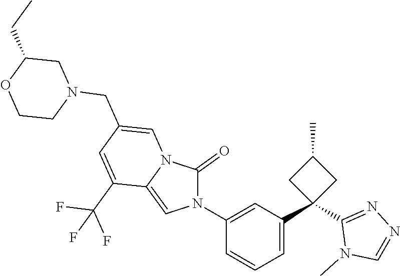 [SMILES: CC[C@@H]1CN(Cc2cc(C(F)(F)F)c3cn(-c4cccc([C@]5(c6nncn6C)C[C@@H](C)C5)c4)c(=O)n3c2)CCO1] |
| 129 | 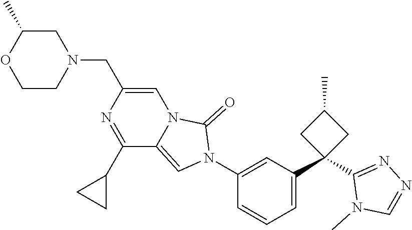 [SMILES: C[C@@H]1CN(Cc2cn3c(=O)n(-c4cccc([C@]5(c6nncn6C)C[C@@H](C)C5)c4)cc3c(C3CC3)n2)CCO1] |
| 130 | 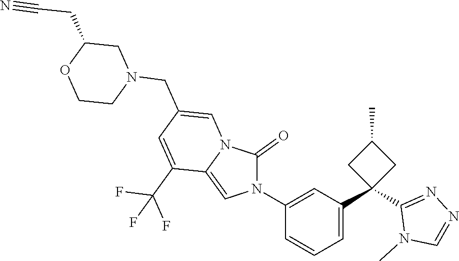 [SMILES: Cn1cnnc1[C@]1(c2cccc(-n3cc4c(C(F)(F)F)cc(CN5CCO[C@H](CC#N)C5)cn4c3=O)c2)C[C@H](C)C1] |
| 131 | 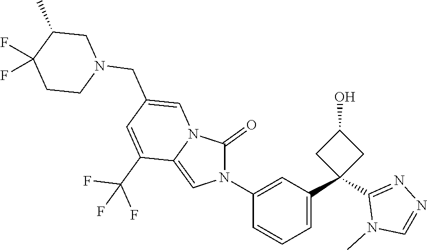 [SMILES: C[C@@H]1CN(Cc2cc(C(F)(F)F)c3cn(-c4cccc([C@]5(c6nncn6C)C[C@@H](O)C5)c4)c(=O)n3c2)CCC1(F)F] |
| 132 | 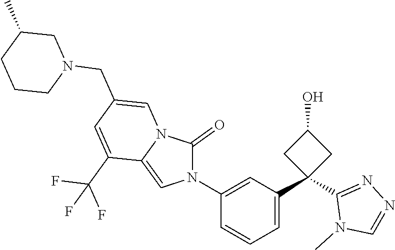 [SMILES: C[C@H]1CCCN(Cc2cc(C(F)(F)F)c3cn(-c4cccc([C@]5(c6nncn6C)C[C@@H](O)C5)c4)c(=O)n3c2)C1] |
| 133 | 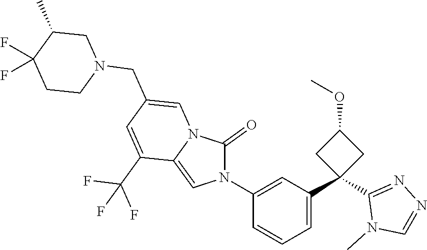 [SMILES: CO[C@H]1C[C@@](c2cccc(-n3cc4c(C(F)(F)F)cc(CN5CCC(F)(F)[C@H](C)C5)cn4c3=O)c2)(c2nncn2C)C1] |
| 134 | 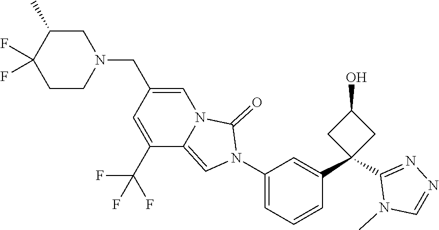 [SMILES: C[C@@H]1CN(Cc2cc(C(F)(F)F)c3cn(-c4cccc([C@]5(c6nncn6C)C[C@H](O)C5)c4)c(=O)n3c2)CCC1(F)F] |
| 135 | 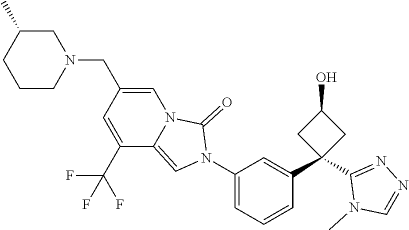 [SMILES: C[C@H]1CCCN(Cc2cc(C(F)(F)F)c3cn(-c4cccc([C@]5(c6nncn6C)C[C@H](O)C5)c4)c(=O)n3c2)C1] |
| 136 | 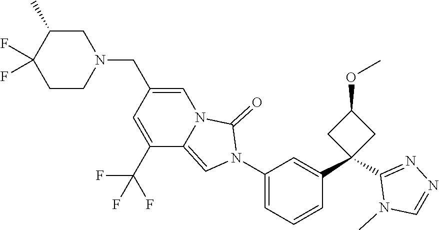 [SMILES: CO[C@H]1C[C@](c2cccc(-n3cc4c(C(F)(F)F)cc(CN5CCC(F)(F)[C@H](C)C5)cn4c3=O)c2)(c2nncn2C)C1] |
| 137 | 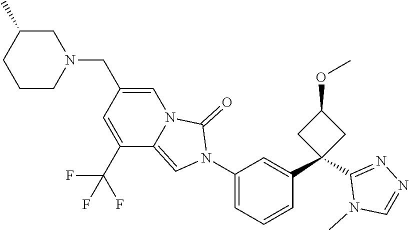 [SMILES: CO[C@H]1C[C@](c2cccc(-n3cc4c(C(F)(F)F)cc(CN5CCC[C@H](C)C5)cn4c3=O)c2)(c2nncn2C)C1] |
| 138 | 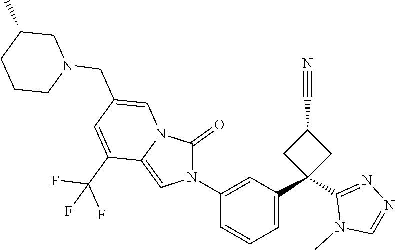 [SMILES: C[C@H]1CCCN(Cc2cc(C(F)(F)F)c3cn(-c4cccc([C@]5(c6nncn6C)C[C@@H](C#N)C5)c4)c(=O)n3c2)C1] |
| 139 | 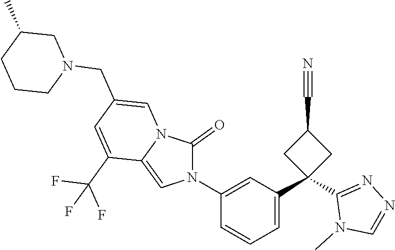 [SMILES: C[C@H]1CCCN(Cc2cc(C(F)(F)F)c3cn(-c4cccc([C@]5(c6nncn6C)C[C@H](C#N)C5)c4)c(=O)n3c2)C1] |
| 140 | 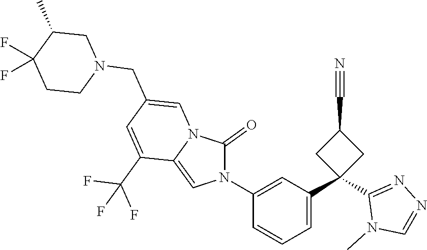 [SMILES: C[C@@H]1CN(Cc2cc(C(F)(F)F)c3cn(-c4cccc([C@]5(c6nncn6C)C[C@H](C#N)C5)c4)c(=O)n3c2)CCC1(F)F] |
| 141 | 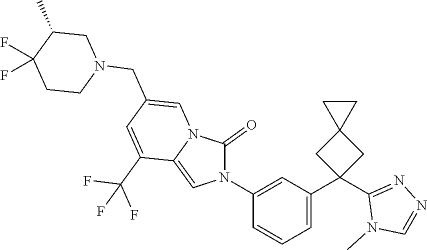 [SMILES: C[C@@H]1CN(Cc2cc(C(F)(F)F)c3cn(-c4cccc(C5(c6nncn6C)CC6(CC6)C5)c4)c(=O)n3c2)CCC1(F)F] |
| 142 | 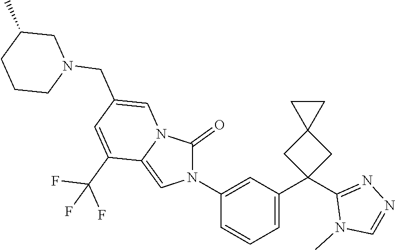 [SMILES: C[C@H]1CCCN(Cc2cc(C(F)(F)F)c3cn(-c4cccc(C5(c6nncn6C)CC6(CC6)C5)c4)c(=O)n3c2)C1] |
| 143 | 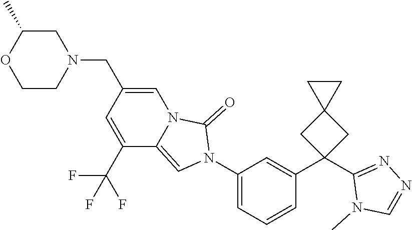 [SMILES: C[C@@H]1CN(Cc2cc(C(F)(F)F)c3cn(-c4cccc(C5(c6nncn6C)CC6(CC6)C5)c4)c(=O)n3c2)CCO1] |
| 144 | 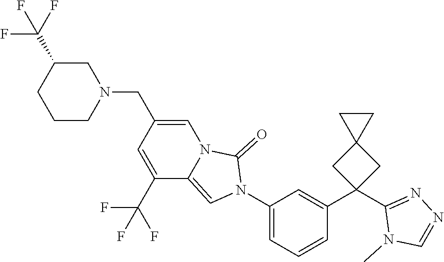 [SMILES: Cn1cnnc1C1(c2cccc(-n3cc4c(C(F)(F)F)cc(CN5CCC[C@H](C(F)(F)F)C5)cn4c3=O)c2)CC2(CC2)C1] |
| 145 | 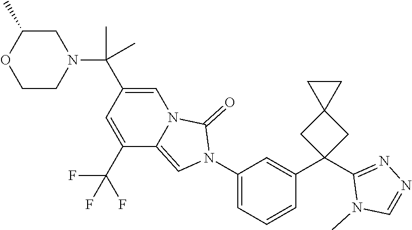 [SMILES: C[C@@H]1CN(C(C)(C)c2cc(C(F)(F)F)c3cn(-c4cccc(C5(c6nncn6C)CC6(CC6)C5)c4)c(=O)n3c2)CCO1] |
| 146 | 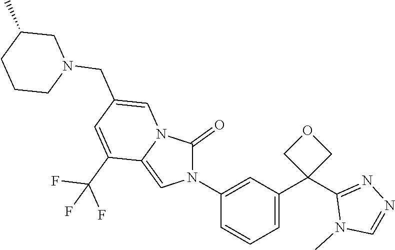 [SMILES: C[C@H]1CCCN(Cc2cc(C(F)(F)F)c3cn(-c4cccc(C5(c6nncn6C)COC5)c4)c(=O)n3c2)C1] |
| 147 | 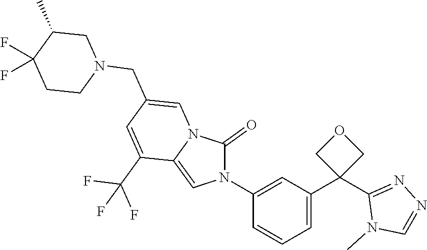 [SMILES: C[C@@H]1CN(Cc2cc(C(F)(F)F)c3cn(-c4cccc(C5(c6nncn6C)COC5)c4)c(=O)n3c2)CCC1(F)F] |
| 148 | 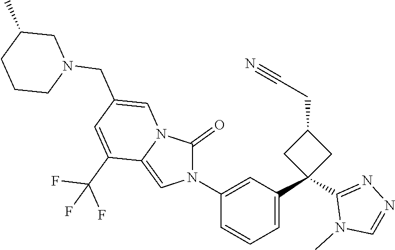 [SMILES: C[C@H]1CCCN(Cc2cc(C(F)(F)F)c3cn(-c4cccc([C@]5(c6nncn6C)C[C@@H](CC#N)C5)c4)c(=O)n3c2)C1] |
| 149 | 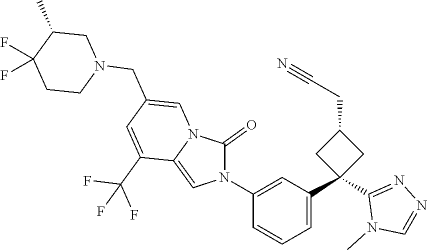 [SMILES: C[C@@H]1CN(Cc2cc(C(F)(F)F)c3cn(-c4cccc([C@]5(c6nncn6C)C[C@@H](CC#N)C5)c4)c(=O)n3c2)CCC1(F)F] |
| 150 | 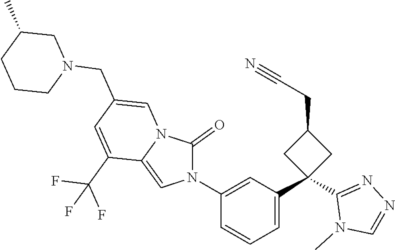 [SMILES: C[C@H]1CCCN(Cc2cc(C(F)(F)F)c3cn(-c4cccc([C@]5(c6nncn6C)C[C@H](CC#N)C5)c4)c(=O)n3c2)C1] |
| 151 | 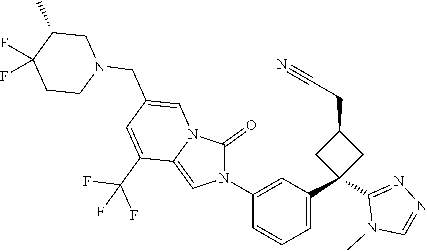 [SMILES: C[C@@H]1CN(Cc2cc(C(F)(F)F)c3cn(-c4cccc([C@]5(c6nncn6C)C[C@H](CC#N)C5)c4)c(=O)n3c2)CCC1(F)F] |
| 152 | 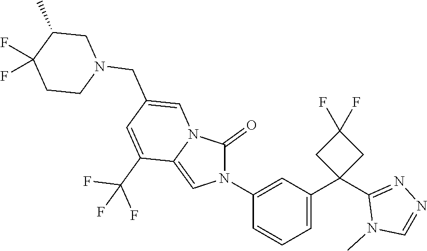 [SMILES: C[C@@H]1CN(Cc2cc(C(F)(F)F)c3cn(-c4cccc(C5(c6nncn6C)CC(F)(F)C5)c4)c(=O)n3c2)CCC1(F)F] |
| 153 | 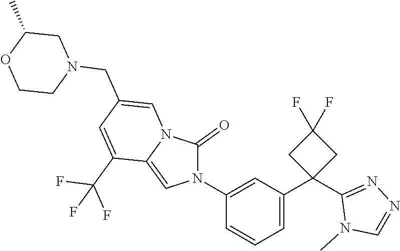 [SMILES: C[C@@H]1CN(Cc2cc(C(F)(F)F)c3cn(-c4cccc(C5(c6nncn6C)CC(F)(F)C5)c4)c(=O)n3c2)CCO1] |
| 59′ | 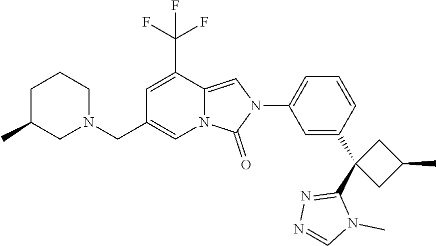 |
| 72′ | 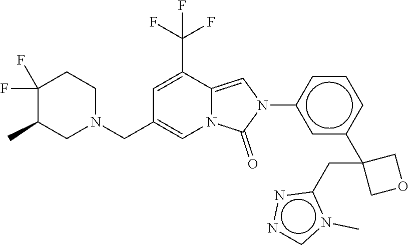 |
| 60′ | 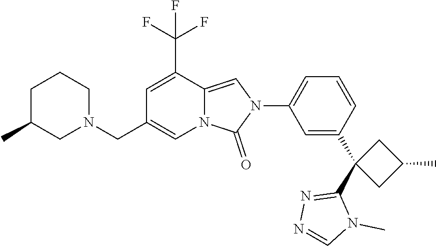 |
| 73′ | 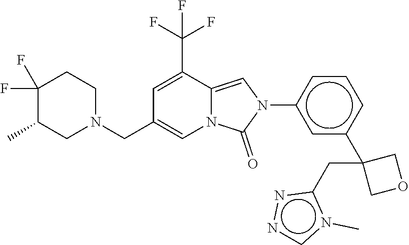 |
| 189′ | 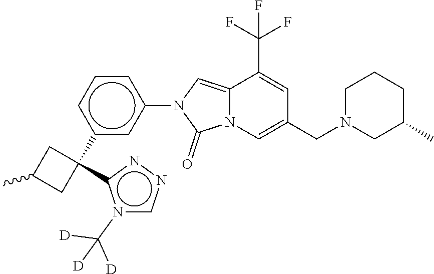 |
| 416′ | 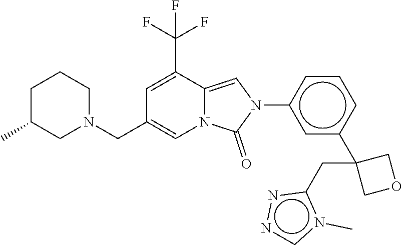 |
| 190′ | 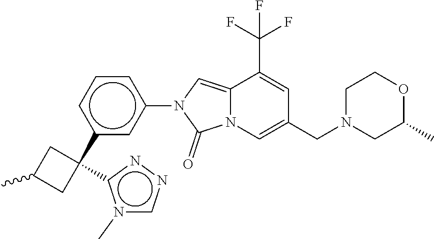 |
| 301′ | 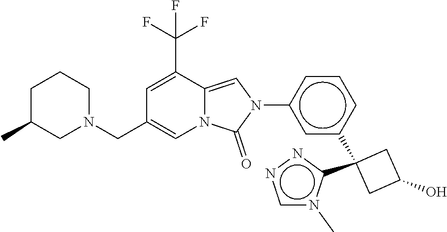 |
| 213′ | 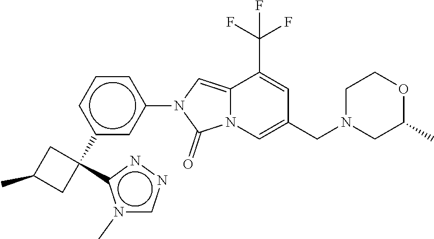 |
| 318′ | 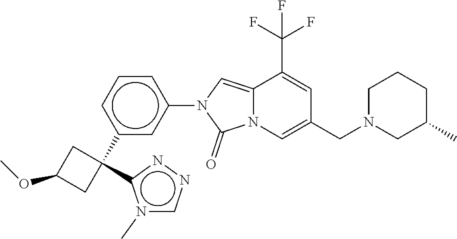 |
| 214′ | 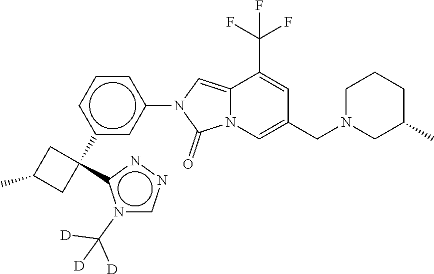 |
| 319′ | 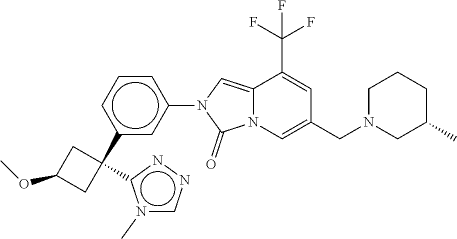 |
| 255′ | 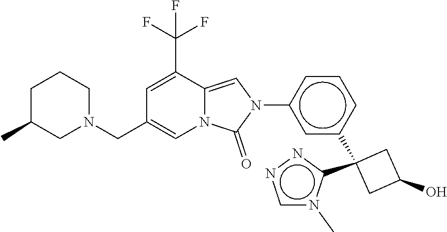 |
| 439′ | 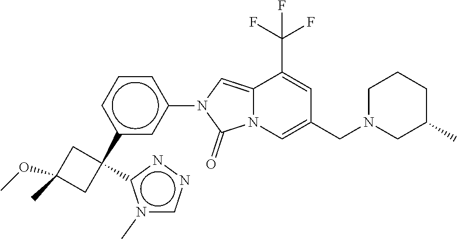 |
| 341′ | 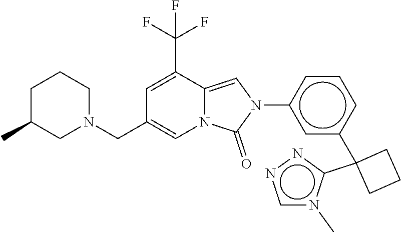 |
| 440′ | 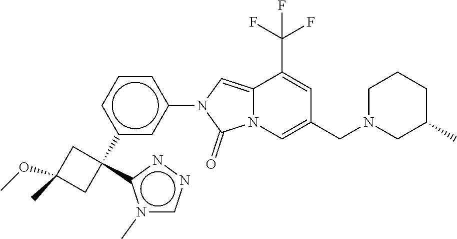 |
| 417′ | 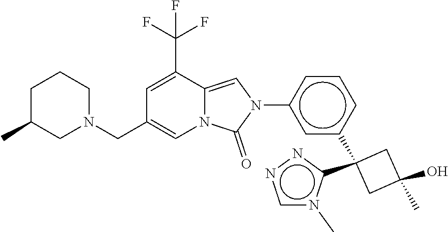 |
| 506′_P1 | 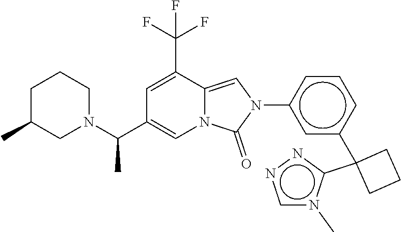 |
| 418′ | 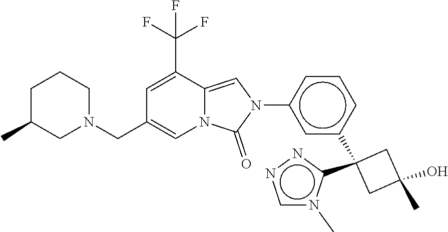 |
| 506′_P2 | 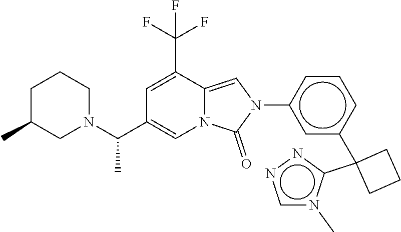 |
| 433′ | 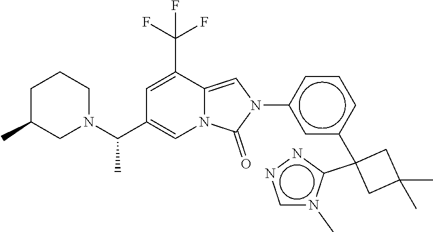 |

### 4. Preparation of Compounds

The compounds provided herein can be prepared, isolated or obtained by any method apparent to those of skill in the art. Exemplary methods of preparation are described in WO 2006/136502, WO 2007/010217, WO 2008/070305, WO 2019/148005, WO 2020/210508, WO 2020/236654, WO 2020/264398, WO 2021/021761, WO 2021/243471, WO 2022/169997, WO 2022/169998, WO 2022/217276, WO 2022/221704, WO 2022/272248, and WO 2023/036330, the contents of which are hereby incorporated by reference in their entireties.

### 5. Pharmaceutical Compositions and Methods of Administration

The CBL inhibitor compounds provided herein can be formulated into pharmaceutical compositions using methods available in the art and those disclosed herein. In particular embodiments, the CBL inhibitor compound is formulated in a pharmaceutical composition comprising the compound and one or more pharmaceutically acceptable carriers, diluents or excipients.

The methods provided herein encompass administering pharmaceutical compositions comprising a CBL compound and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In certain embodiments, a pharmaceutical composition provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), The Pharmaceutical Press, incorporated by reference in its entirety.

In certain embodiments, the pharmaceutical composition comprises an anti-foaming agent. Any suitable anti-foaming agent may be used. In certain embodiments, the anti-foaming agent is selected from an alcohol, an ether, an oil, a wax, a silicone, a surfactant, and combinations thereof. In certain embodiments, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof

In certain embodiments, the pharmaceutical composition comprises a co-solvent. Illustrative examples of co-solvents include ethanol, poly(ethylene) glycol, butylene glycol, dimethylacetamide, glycerin, and propylene glycol.

In certain embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In certain embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In certain embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In certain embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In certain embodiments, the pharmaceutical composition comprises a solvent. In certain embodiments, the solvent is saline solution, such as a sterile isotonic saline solution or dextrose solution. In certain embodiments, the solvent is water for injection.

In certain embodiments, the pharmaceutical compositions are in a particulate form, such as a microparticle or a nanoparticle. Microparticles and nanoparticles may be formed from any suitable material, such as a polymer or a lipid. In certain embodiments, the microparticles or nanoparticles are micelles, liposomes, or polymersomes.

Further provided herein are anhydrous pharmaceutical compositions and dosage forms comprising therapeutic agent, since, In certain embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopeia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amounts of the CBL compound or composition which will be effective in the prevention or treatment of a disorder or one or more symptoms thereof will vary with the nature and severity of the disease or condition, and the route by which the agents are administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram).

In certain embodiments, the dosage of the CBL compound provided herein, based on weight of the compound, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the com CBL compound is 0.1 mg to 1000 mg, 0.1 mg to 900 mg, 0.1 mg to 800 mg, 0.1 mg to 750 mg, 0.1 mg to 700 mg, 0.1 mg to 600 mg, 0.1 mg to 500 mg, 0.1 mg to 400 mg, 0.1 mg to 300 mg, 0.1 mg to 250 mg, 0.1 mg to 200 mg, 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, the CBL inhibitor compound is administered in combination with one or more second agents. The second agent can be any second agent deemed suitable to the person of skill. In certain embodiments, the second agent is an anti-PD1 antibody. In certain embodiments, the second agent is pembrolizumab. In certain embodiments, the second agent is nivolumab. In certain embodiments, the second agent is cemiplimab. In certain embodiments, the second agent is atezolizumab. In certain embodiments, the second agent is avelumab. In certain embodiments, the second agent is durvalumab. In certain embodiments, the second agent is a PARP inhibitor. In certain embodiments, the second agent is olaparib. In certain embodiments, the second agent is talazoparib. In certain embodiments, the second agent is niraparib. In certain embodiments, the second agent is a chemotherapeutic. In certain embodiments, the second agent is paclitaxel. In certain embodiments, the second agent is docetaxel. In certain embodiments, the second agent is a cell therapy.

The dose of either agent can be administered according to a suitable schedule, for example, once, two times, three times, or four times weekly. It may be necessary to use dosages of the agents outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the agents provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an agent provided herein followed by one or more maintenance doses.

In certain embodiments, a dose of an agent provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### 6. Therapeutic Applications

The combinations provided herein may be useful for the treatment of any disease or condition involving abnormal cell growth or proliferation. In certain embodiments, the disease or condition is a disease or condition that can benefit from treatment with CBL inhibitor compound. In certain embodiments, the disease or condition is a cancer. In certain embodiments, the disease or condition is a solid tumor. In certain embodiments, the disease or condition is a hematological cancer.

Any suitable cancer may be treated with combinations provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In certain embodiments, the disease to be treated with the combinations provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In certain embodiments, the disease is ovarian cancer. In certain embodiments, the disease is breast cancer. In certain embodiments, the disease is triple-negative breast cancer (TNBC). In certain embodiments, the disease is lung cancer. In certain embodiments, the disease is non-small cell lung cancer (NSCLC). In certain embodiments, the disease is head and neck cancer. In certain embodiments, the disease is renal cell carcinoma. In certain embodiments, the disease is brain carcinoma. In certain embodiments, the disease is endometrial cancer.

### 7. Kits

In certain embodiments, a compound or combination provided herein is provided in the form of a kit, i.e., a packaged combination of reagents in predetermined amounts with instructions for performing a procedure. In other embodiments, the procedure is a therapeutic procedure. In certain embodiments, the kit comprises a CBL inhibitor compound, or a composition thereof, and instructions for use in combination with detection of one or more CBL biomarkers described herein. In certain embodiments, the kit comprises a CBL inhibitor compound and one or more reagents capable of detecting a CBL biomarker described herein. In certain embodiments, the kit comprises a CBL inhibitor compound and one or more reagents capable of detecting pHS1. In certain embodiments, the kit comprises a CBL inhibitor compound and one or more reagents capable of detecting pPLCγ2. In certain embodiments, the kit comprises a CBL inhibitor compound and one or more reagents capable of detecting pZAP70.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hrs (hours); min (minutes); MS (mass spectrometry); ESI (electrospray ionization); TLC (thin layer chromatography); HPLC (high pressure liquid chromatography); THF (tetrahydrofuran); CDCl₃ (deuterated chloroform); AcOH (acetic acid); DCM (dichloromethane); DMSO (dimethylsulfoxide); DMSO-d₆ (deuterated dimethylsulfoxide); EtOAc (ethyl acetate); MeOH (methanol); and BOC (t-butyloxycarbonyl).

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

### Example 1: Phosphorylated HS1, PLCγ2 and ZAP70 Biomarker Assessment

Cbl-b has been characterized as a negative regulator of the adaptive immune response (Bachmaier et al., 2000, Nature 403(6766):211-6; Chiang et al., 2000, Nature 403(6766):216-20). Both T cell receptor (TCR) signaling and B cell receptor (BCR) signaling are modulated by Cbl-b mediated ubiquitination. Compound 23 is a small molecule inhibitor of Cbl-b. In order to identify proximal biomarkers of Cbl-b inhibition in immune cells, a panel of >100 phosphorylated proteins were screened in human PBMCs treated with Cbl-b inhibitor. The effect of Cbl-b inhibition was assessed across multiple cellular signaling networks, and three candidate proximal biomarkers of Cbl-b inhibition were identified, including phosphorylated Hematopoietic lineage cell-specific protein (HS1), phosphorylated phospholipase C2 (PLCγ2) and phosphorylated zeta chain protein kinase 70 (ZAP70) (Rhee et al., 2001, Annual Review of Biochemistry. 70:281-312; Fu et al., 2010, J. Experimental Med. 207(2):309-318).

HS1 binds the actin cytoskeleton and regulates TCR activation through the immune synapse (Yamanashi et al., 1993, Proc. Natl. Acad. Sci. USA 90(8):3631-5; Scaife et al., 2003, J. Cell Science 116(3): 463-473; Gomez et al., 2006, Immunity 24(6):741-752; Carrizosa et al., 2009, J. Immunol. 183(11):7352-61). PLCγ2 and ZAP70 are expressed in T cells and activated upon TCR stimulation. Increased activation of phosphorylated HS1, PLCγ2, and ZAP70 after Cbl-b inhibition identifies a central function for Cbl-b in regulating proximal T cell activation and receptor tyrosine kinase (RTK) signaling in the immune synapse.

To assess these proximal biomarkers of Cbl-b inhibition on CD8⁺ T cells, pharmacodynamic analysis was performed in conjunction with a pharmacokinetic study conducted at Charles River Laboratories. Circulating blood was obtained from cynomolgus monkeys treated with 0.3, 1, 3 or 10 mg/kg of Compound 23 after a single dose and after 3 doses. The response to Cbl-b inhibition on proximal biomarkers (pHS1, pPLCγ2, and pZAP70) was measured in CD8⁺ T cells using flow cytometry. This technique is highly quantitative and can measure multiple phosphorylation markers in different cell populations within the same sample.

Compound 23 was formulated in vehicle (0.2% Tween 80 in DI water+1 eq HCL) and aliquoted into individual (not serially diluted) dosing solutions of 0.3, 1, 3 and 10 mg/mL by stirring for 5-10 min, vortexing, and sonicating again as necessary to produce an even suspension that was stirred constantly at 4° C. to achieve and maintain a homogenous suspension. To prepare vehicle, 0.2% Tween 80 in deionized (DI) water, water was measured into a beaker with continuous stirring. Tween 80 was added sequentially, and stirring continued until the solution dissolved; the solution was stored at 2-8° C.

Whole Blood Collection for Pharmacodynamic Analysis

For pharmacodynamic analysis, whole blood was obtained from cynomolgus monkeys and used for flow cytometry assays to evaluate phosphorylated biomarkers, pHS1, pPLCγ2, and pZAP70, in circulating CD8⁺ T cells.

On the day of dosing, blood samples were collected via femoral venipuncture in accordance with test facility standard operating procedures. 1 mL/tube of whole blood was collected pre-dose (time 0) and at 0.5, 1, 2, 4, 6, and 24 hours post-dose and added into 2 types of TruCulture tubes (Myriad) for pharmacodynamic analysis. One tube contained media alone and is denoted as “Null”, and the second tube contained 2 μg/mL anti-CD3 and anti-CD28, which were prepared at Nurix prior to the study using cynomolgus specific anti-CD3 (BD Biosciences; SP34-2) and anti-CD28 (Bio X cell; CD28.2) and denoted as “CD3/CD28”. Tubes were shipped by overnight courier on cold pack the day of collection and analyzed for phosphorylated biomarkers of Cbl-b inhibition by flow cytometry.

Pharmacodynamic Analysis of Phosphorylated Biomarkers in Circulating Blood CD8⁺ T Cells Using Flow Cytometry-Based Assay

Following PD collection, upon receipt of TruCulture tubes, each tube was inverted 5-6 times to ensure a mixed suspension of blood. 30 μg of goat anti-mouse IgG (H+L) antibody (Invitrogen) was added to cross-link the stimulation antibodies and induce synchronized cellular stimulation. Cells were stimulated for 60 minutes alongside non-stimulated controls for each time point.

All buffers (Phosflow Lyse/Fix Buffer 5×, Phosflow Perm Buffer I; BD Biosciences) were prepared according to the manufacturer's instructions. After 60 minutes of goat anti-mouse IgG (H+L) antibody stimulation, one volume of blood was mixed with 20 volumes of pre-warmed BD Phosflow Lyse/Fix Buffer (1×) by repeated pipetting (8-10 times), then incubated in a 37° C. water bath for 10 min. Cells were washed and centrifuged at 500×g for 5 min (2×) with PBS. Following centrifugation, cell pellets were resuspended in cold BD Phosflow Perm Buffer I (BD) and incubated at 4° C. for 20 min.

Endogenous Fc receptors were blocked using human BD Fc block (2.5 μg/mL/100 total volume) prepared in 1×PBS for 10 min at 4° C., and cells were stained with Alexafluor700 mouse anti-NHP CD45 (BD Biosciences; 5 μg/mL), Brilliant Violet 605 mouse anti-human CD8 (BD Biosciences; 5 μs/ml), AlexaFluor-647 mouse anti-human phosphorylated Y759-PLCγ2 (BD; 5 μg/ml) and Phycoerythrin (PE) mouse anti-rabbit Y397-HS1 (Cell signaling Technology; 1:50), and AlexaFluor-488 mouse anti-human phosphorylated Y319/Syk (Y352)-ZAP70 (BD; 5 μg/ml) and incubated for 1 hr at 4° C. After 1 hr of incubation, cells were washed twice with Phosflow Perm Buffer I (BD Biosciences) and resuspended in 200 μL PBS for flow cytometric analysis.

Stained cells were run on an Attune NxT Acoustic Focusing Flow Cytometer (Thermo Fisher, cat. No. A29004), and data was analyzed using FlowJo (v10.6.1) and GraphPad Prism (v8.00) software. Single lymphocytes were gated for CD8⁺ cells (CD45⁺/CD8⁺) cells, and population frequencies were calculated for each population. The data is quantified by the percent positive. Using unstimulated pre-dose control for each animal as a baseline, a gate is placed at the 90th percentile for that specific cell population and phosphoprotein. As protein becomes phosphorylated the fluorescence increases resulting in a larger fraction of the cells entering that gate. Each population was evaluated for % positive pY759-PLCγ2, pY397-HS1 levels, pY319/Syk (Y352)-ZAP70 levels.

For acute biomarker response after single dose administration, the fold change of each biomarker at the indicated timepoint post dose was calculated with the following example equation:

[% phosphorylated biomarker of anti-CD3/CD28 stimulated sample−% phosphorylated biomarker of null sample]/% day 1 baseline (T=0 hr)]

For accumulated biomarker response after multi-dose administration, the fold change of each biomarker at indicated timepoint post-dose was calculated with the following example equation:

[% phosphorylated biomarker of anti-CD3/CD28 stimulated sample−% phosphorylated biomarker of null sample]/% day 1 baseline (T=0 hr)]

Prism 8.3.0 (GraphPad Prism) for Windows was used for graphical presentations and statistical analyses. Statistical analyses of the differences between groups were accomplished using two-way ANOVA with Dunnett's multiple comparisons test. Criteria include: ns, not significant, p>0.05; *, significant, 0.01<p≤0.05; **, very significant, 0.001<p≤0.0; ***, highly significant, p≤0.001; ****, extremely significant, p≤0.0001.

Results

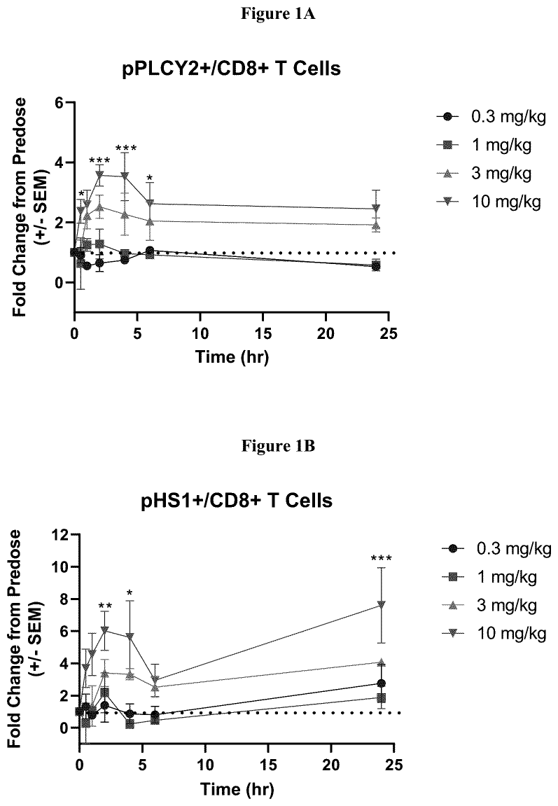

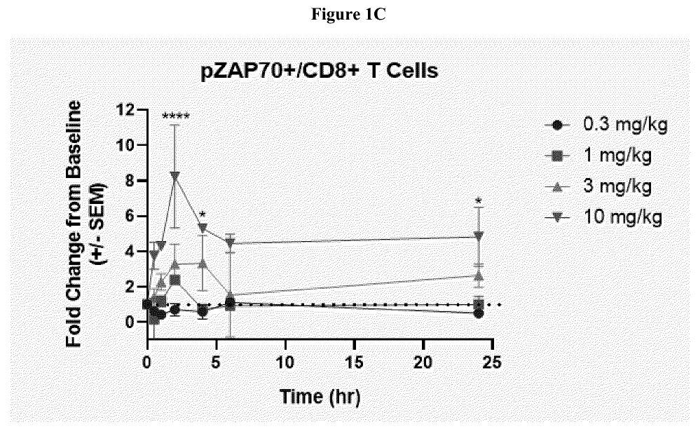

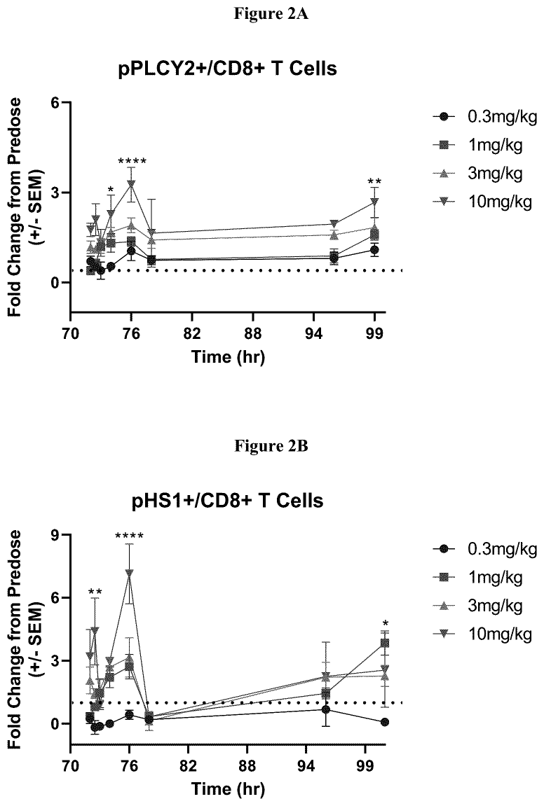

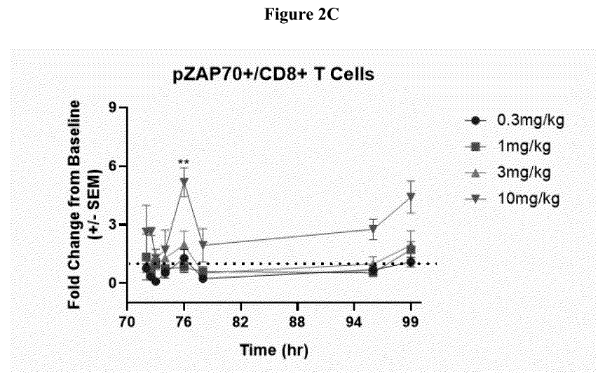

Using a flow cytometry assay, the kinetics of phosphorylated PLCγ2, HS1, and ZAP70 were assessed in circulating CD8⁺ T cells after a single dose (FIGS. 1A, 1B, and 1C) and after 3 days of daily oral dosing (FIGS. 2A, 2B, and 2C) of either 0.3, 1, 3 or 10 mg/kg Compound 23 in cynomolgus monkeys.

In order to understand the acute biomarker response after a single dose of Compound 23 in circulating CD8⁺ T cells, the levels of phosphorylated PLCγ2, HS1, and ZAP70 were assessed. Dose responsive increases in proximal biomarkers were observed post Compound 23 exposure. Single dose oral administration of 10 mg/kg Compound 23 was associated with significant increases in phosphorylated ZAP70, PLCγ2 and HS1 in comparison to baseline. Maximal signal was observed 2 hours post dose for both phosphorylated HS1 and phosphorylated PLCγ2 and phosphorylated ZAP70 (p=0.0046, p=0.0003 and p<0.0001, respectively) (FIGS. 1A, 1B, and 1C). After 24 hours, a significant increase of phosphorylated HS1 and ZAP70 was sustained (p=0.0001, p=0.0309) respectively (FIGS. 1A and 1C). Biomarker responses after a single dose of Compound 23 are summarized in Table 8.

Increased Phosphorylation of PLCγ2, HS1, and ZAP70 after Multiple Doses of Compound 23

In order to understand the pharmacodynamic effects of Compound 23 in circulating CD8⁺ T cells after 3 days of daily dosing, the levels of phosphorylated PLCγ2, ZAP70, and HS1 were assessed (FIGS. 2A, 2B, and 2C). Dose responsive increases in proximal biomarkers were observed post Compound 23 exposure. Multiple doses of 10 mg/kg Compound 23 were associated with significant increases in phosphorylated PLCγ2, ZAP70, and HS1 in comparison to baseline. Peak increases of phosphorylated PLCγ2, ZAP70, and HS1 were observed after 76 hours (4 hours post dose) of 10 mg/kg Compound 23 (pHS1 and pPLCγ2 p<0.0001; pZAP70 p=0.0078) in comparison to pre-treatment baseline.

FIG. 1A provides single dose Compound 23 in cynomolgus monkeys induces phosphorylated PLCγ2 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 1, fold change of % phosphorylated PLCγ2 normalized to baseline (T=0) was quantitated in CD8⁺ T cells (CD45⁺/CD8⁺) using flow cytometry. Graph represents the mean (±SEM) fold change of phosphorylated PLCγ2 at each time point after correcting for background in unstimulated cells and normalizing to baseline (T=0) phosphorylated PLCγ2 levels. Dashed line indicates normalized baseline (T=0). Statistical analyses of the differences between baseline and each dosing group were accomplished using two-way ANOVA with Dunnett's multiple comparisons test and are indicated above each data point.

FIG. 1B provides single dose Compound 23 in cynomolgus monkeys induces phosphorylated HS1 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 1, fold change of % phosphorylated HS1 normalized to baseline (T=0) was quantitated in T cells (CD45⁺/CD8⁺) using flow cytometry. Graph represents the mean (±SEM) fold change of phosphorylated HS1 at each time point after correcting for background in unstimulated cells and normalizing to baseline (T=0) phosphorylated HS1 levels. Dashed line indicates normalized baseline (T=0). Statistical analyses of the differences between baseline and each dosing group were accomplished using two-way ANOVA with Dunnett's multiple comparisons test and are indicated above each data point.

FIG. 1C provides single dose Compound 23 in cynomolgus monkeys induces phosphorylated ZAP70 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 1, fold change of % phosphorylated HS1 normalized to baseline (T=0) was quantitated in T cells (CD45⁺/CD8⁺) using flow cytometry. Graph represents the mean (±SEM) fold change of phosphorylated ZAP70 at each time point after correcting for background in unstimulated cells and normalizing to baseline (T=0) phosphorylated ZAP70 levels. Dashed line indicates normalized baseline (T=0). Statistical analyses of the differences between baseline and each dosing group were accomplished using two-way ANOVA with Dunnett's multiple comparisons test and are indicated above each data point.

FIG. 2A provides multiple doses of Compound 23 in cynomolgus monkeys induces phosphorylated PLCγ2 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 3, fold change of % phosphorylated PLCγ2 normalized to baseline (T=0) was quantitated in T cells (CD45⁺/CD8⁺) using flow cytometry. Graph represents the mean (±SEM) fold change of phosphorylated PLCγ2 at each time point after correcting for background in unstimulated cells and normalizing to baseline (T=0) phosphorylated PLCγ2 levels. Dashed line indicates normalized baseline (T=0). Statistical analyses of the differences between baseline and each dosing group were accomplished using two-way ANOVA with Dunnett's multiple comparisons test and are indicated above each data point.

FIG. 2B provides multiple doses of Compound 23 in cynomolgus monkeys induces phosphorylated HS1 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 3, fold change of % phosphorylated HS1 normalized to baseline (T=0) was quantitated in T cells (CD45⁺/CD8⁺) using flow cytometry. Graph represents the mean (±SEM) fold change of phosphorylated HS1 at each time point after correcting for background in unstimulated cells and normalizing to baseline (T=0) phosphorylated HS1 levels. Dashed line indicates normalized baseline (T=0). Statistical analyses of the differences between baseline and each dosing group were accomplished using two-way ANOVA with Dunnett's multiple comparisons and are indicated above each data point.

FIG. 2C provides multiple doses of Compound 23 in cynomolgus monkeys induces phosphorylated ZAP70 upon stimulation of circulating CD8⁺ T cells. Cynomolgus monkeys were orally administered either 0.3, 1, 3 or 10 mg/kg Compound 23 and whole blood was collected into 2 types of TruCulture tubes (“Null” for aliquot 1; “CD3/CD28” for aliquot 2) at each indicated timepoints pre and post dosing (n=3/group) for 3 days. On day 3, fold change of % phosphorylated ZAP70 normalized to baseline (T=0) was quantitated in T cells (CD45⁺/CD8⁺) using flow cytometry. Graph represents the mean (±SEM) fold change of phosphorylated ZAP70 at each time point after correcting for background in unstimulated cells and normalizing to baseline (T=0) phosphorylated ZAP70 levels. Dashed line indicates normalized baseline (T=0). Statistical analyses of the differences between baseline and each dosing group were accomplished using two-way ANOVA with Dunnett's multiple comparisons and are indicated above each data point.

### CONCLUSIONS

These data confirm specific proximal pHS1, pPLCγ2, and pZAP70 biomarker changes in circulating CD8⁺ T cells following administration of the Cbl-b inhibitor, Compound 23, to cynomolgus NHP.

### Example 2: Phosphorylated HS1, PLCγ2, and ZAP70 Biomarker Assessment

Cbl-b has been characterized as a negative regulator of the adaptive immune response (Bachmaier et al., 2000, Nature 403(6766):211-6; Chiang et al., 2000, Nature 403(6766):216-20). Both T cell receptor (TCR) signaling and B cell receptor (BCR) signaling are modulated by Cbl-b mediated ubiquitination. Compound 23 is a small molecule inhibitor of Cbl-b. In order to identify proximal biomarkers of Cbl-b inhibition in immune cells, a panel of >100 phosphorylated proteins were screened in human PBMCs treated with Cbl-b inhibitor. The effect of Cbl-b inhibition was assessed across multiple cellular signaling networks, and three candidate proximal biomarkers of Cbl-b inhibition were identified, including phosphorylated Hematopoietic lineage cell-specific protein (HS1), phosphorylated phospholipase C2 (PLCγ2) and phosphorylated zeta chain protein kinase 70 (ZAP70) (Rhee et al., 2001, Annual Review of Biochemistry. 70:281-312; Fu et al., 2010, J. Experimental Med. 207(2):309-318).

PLCγ2 is expressed in T cells and activated upon TCR stimulation. Increased activation of pHS1, pPLCγ2, and pZAP70 after Cbl-b inhibition identifies a central function for Cbl-b in regulating proximal T cell activation and receptor tyrosine kinase (RTK) signaling in the immune synapse.

To assess these candidate clinical proximal biomarkers of Cbl-b inhibition on T cells, pharmacodynamic analysis was performed. Whole blood was obtained from human donors and incubated with increasing concentrations of Compound 23. The response to Cbl-b inhibition on phosphorylated HS1, PLCγ2, and ZAP70 was measured in CD8⁺ T cells using flow cytometry. This technique is highly quantitative and can measure multiple phosphorylation markers in different cell populations within the same sample.

For pharmacodynamic analysis, whole blood was obtained from six human donors (BioIVT) and used for flow cytometry assays to evaluate phosphorylated biomarkers HS1, PLCγ2, and ZAP70 in CD8⁺ T cells.

Whole blood was collected in a collection tube coated with sodium heparin. Compound dilution series (7-points, 0.01, 0.1, 1, 10, 50, 100, 1000 nM) were prepared in media and added to cells. Blood samples were pre-incubated with compound for two hours at 37° C. at concentrations ranging from 0.01-1000 nM with a final DMSO concentration of 0.05%.

After incubation with compound, 100 μL blood from each indicated concentration per donor was subsequently transferred to a 2.4 mL deep well Master Block plate for pharmacodynamic analysis. 0.2 μg/mL of mouse anti-human CD3 and 0.33 μg/mL of mouse anti-human CD28 was added to each well for 5 minutes. Next, 30 μg of goat anti-mouse IgG (H+L) antibody (Invitrogen) was added to cross-link the stimulation antibodies and induce synchronized cellular stimulation. Cells were stimulated alongside non-stimulated & stimulated DMSO control for 60 minutes.

All buffers (Phosflow Lyse/Fix Buffer 5×, Phosflow Perm Buffer I; BD Biosciences) were prepared, according to the manufacturer's instructions. After 60 minutes of antibody stimulation, one volume of blood was mixed with 20 volumes of pre-warmed BD Phosflow Lyse/Fix Buffer (1λ) by repeated pipetting (8-10 times), and then transferred to a 37° C. water bath for 10 min. Cells were washed and centrifuged at 500×g for 5 min (2λ) with PBS. Following centrifugation, cell pellets were resuspended in cold BD Phosflow Perm Buffer I (BD) and incubated at 4° C. for 20 min.

Endogenous Fc receptors were blocked using human BD Fc block (2.5 μg/mL/100 total volume) prepared in 1×PBS for 10 min at 4° C., and cells were stained with Alexafluor700 mouse anti-NHP CD45 (BD Biosciences; 5 μg/mL), Brilliant Violet 605 mouse anti-human CD8 (BD Biosciences; 5 μs/ml), AlexaFluor-647 mouse anti-human phosphorylated Y759-PLCγ2 (BD; 5 μg/ml) and Phycoerythrin (PE) mouse anti-rabbit Y397-HS1 (Cell signaling Technology; 1:50), and AlexaFluor-488 mouse anti-human phosphorylated Y319/Syk (Y352)-ZAP70 (BD; 5 μg/ml) and incubated for 1 hr at 4° C. After 1 hr of incubation, cells were washed twice with Phosflow Perm Buffer I (BD Biosciences) and resuspended in 200 μL PBS for flow cytometric analysis.

Stained cells were run on an Attune NxT Acoustic Focusing Flow Cytometer (Thermo Fisher, cat. No. A29004), and data was analyzed using FlowJo (v10.6.1) and GraphPad Prism (v8.00) software. Single lymphocytes were gated for CD8⁺ cells (CD45⁺/CD8⁺) cells, and population frequencies were calculated for each population. The data is quantified by the percent positive. Using unstimulated pre-dose control for each animal as a baseline, a gate is placed at the 90th percentile for that specific cell population and phosphoprotein. As protein becomes phosphorylated the fluorescence increases resulting in a larger fraction of the cells entering that gate. Each population was evaluated for % positive pY759-PLCγ2, pY397-HS1 levels, pY319/Syk (Y352)-ZAP70 levels.

Using a flow cytometry assay, the kinetics of phosphorylated HS1, PLCγ2 and ZAP70 were assessed in human CD8⁺ T cells from whole blood. Compound 23 induced a dose-dependent increase in phosphorylated HS1, PLCγ2, and ZAP70 in the presence of anti-CD3 and anti-CD28 stimulation (FIGS. 1-2). Average mean EC50±standard deviation calculated for phosphorylated PLCγ2, phosphorylated HS1, and phosphorylated ZAP70 were 74±4.95 nM and 89±7.91 nM and 120±8.97 nM, respectively. pPLCγ2 and pZAP70 have baseline phosphorylation in the absence of Compound 23, suggesting there is an alternative pathway stimulating this marker in the absence of Cbl-b inhibition. Although there was phosphorylation of PLCγ2 and ZAP70 at baseline, increased Compound 23 concentration was associated with additional increases in phosphorylation of PLCγ2 and ZAP70. Increased Compound 23 concentration was also associated with increased phosphorylation of HS1. Compared to PLCγ2 and ZAP70, HS1 had lower baseline phosphorylation, less variability between donors, and a greater dynamic range between baseline and maximal phosphorylation in the presence of high Compound 23 concentration, and thus appears to be a more selective proximal biomarker.

These data show proximal biomarker changes with increasing Compound 23 concentration in whole blood from six human donors stimulated with anti-CD3 and anti-CD28 and demonstrate the pharmacodynamic effects of Compound 23 in vitro using a flow cytometry-based assay.

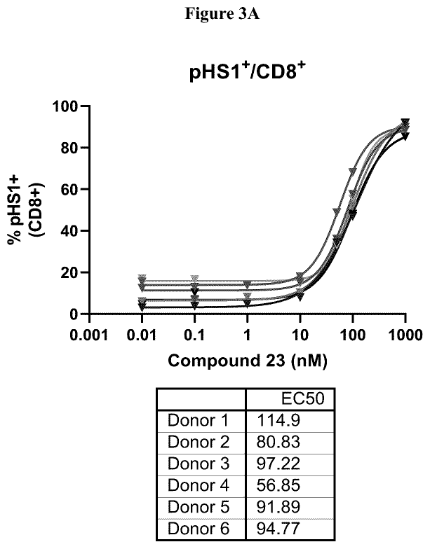

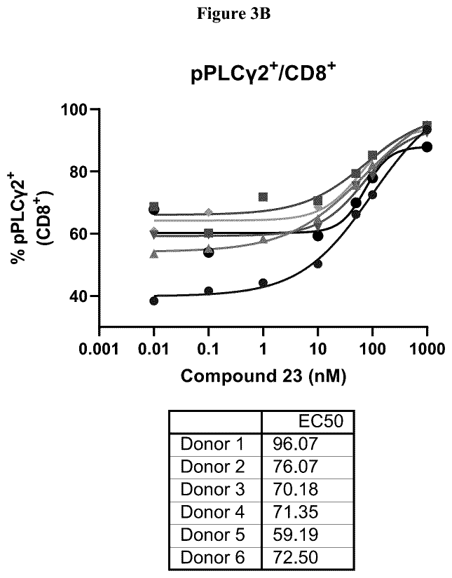

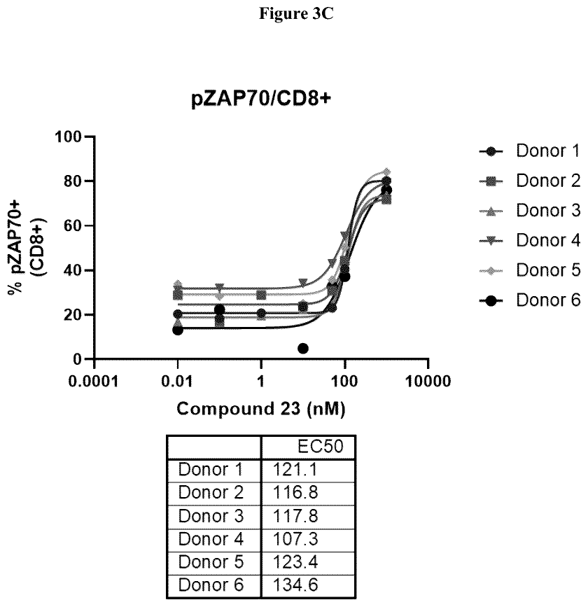

FIG. 3A provides Compound 23 inducing phosphorylated HS1 upon stimulation in vitro in human CD8⁺ T cells from whole blood. Whole blood from six human donors collected in sodium heparin anti-coagulant was treated with increasing concentrations of Compound 23 (0.01-1000 nM) for 2 hrs at 37° C. Compound treated blood was incubated with 0.2 μg/mL anti-CD3 & anti-CD28 for 5 mins and subsequently stimulated with crosslinker for 60 minutes. % phosphorylated HS1 was quantitated in T cells (CD8⁺) using flow cytometry.

FIG. 3B provides Compound 23 inducing phosphorylated PLCγ2 upon stimulation in vitro in human CD8⁺ T cells from whole blood. Whole blood from six human donors collected in sodium heparin anti-coagulant was treated with increasing concentrations of Compound 23 (0.01-1000 nM) for 2 hrs at 37° C. Compound treated blood was incubated with 0.2 μg/mL anti-CD3 & anti-CD28 for 5 mins and subsequently stimulated with crosslinker for 60 minutes. % phosphorylated PLCγ2 was quantitated in T cells (CD8+) using flow cytometry.

FIG. 3C provides Compound 23 inducing phosphorylated ZAP70 upon stimulation in vitro in human CD8⁺ T cells from whole blood. Whole blood from six human donors collected in sodium heparin anti-coagulant was treated with increasing concentrations of Compound 23 (0.01-1000 nM) for 2 hrs at 37° C. Compound treated blood was incubated with 0.2 μg/mL anti-CD3 & anti-CD28 for 5 mins and subsequently stimulated with crosslinker for 60 minutes. % phosphorylated ZAP70 was quantitated in T cells (CD8+) using flow cytometry.

### Example 3: Phosphorylated HS1, PLCγ2, and ZAP70 Biomarker Assessment

Cbl-b has been characterized as a negative regulator of the adaptive immune response (Bachmaier et al., 2000, Nature 403(6766):211-6; Chiang et al., 2000, Nature 403(6766):216-20). Both T cell receptor (TCR) signaling and B cell receptor (BCR) signaling are modulated by Cbl-b mediated ubiquitination. Compound 23 is a small molecule inhibitor of Cbl-b. In order to identify proximal biomarkers of Cbl-b inhibition in immune cells, a panel of >100 phosphorylated proteins were screened in human PBMCs treated with Cbl-b inhibitor. The effect of Cbl-b inhibition was assessed across multiple cellular signaling networks, and three candidate proximal biomarkers of Cbl-b inhibition were identified, including phosphorylated Hematopoietic lineage cell-specific protein (HS1), phosphorylated phospholipase C2 (PLCγ2) and phosphorylated zeta chain protein kinase 70 (ZAP70) (Rhee et al., 2001, Annual Review of Biochemistry. 70:281-312; Fu et al., 2010, J. Experimental Med. 207(2):309-318). PLCγ2 and ZAP70 are expressed in T cells and activated upon TCR stimulation. Increased activation of phosphorylated HS1, ZAP70, and PLCγ2 after Cbl-b inhibition identifies a central function for Cbl-b in regulating proximal T cell activation and receptor tyrosine kinase (RTK) signaling in the immune synapse.

To assess these candidate clinical proximal biomarkers of Cbl-b inhibition on T cells, pharmacodynamic analysis was performed. Whole blood was obtained from cynomolgus macaque donors, then incubated with increasing concentrations of Compound 23. The response to Cbl-b inhibition on phosphorylated HS1, ZAP70 and PLCγ2 was measured in CD8⁺ T cells using flow cytometry. This technique is highly quantitative and can measure multiple phosphorylation markers in different cell populations within the same sample.

DMSO (Dimethyl sulfoxide) was used to reconstitute Compound 23. Final concentration of DMSO was 0.05% in media for in vitro assays.

For pharmacodynamic analysis, whole blood obtained from 6 cynomolgus macaque animal donors (BioIVT) and used for flow cytometry assays to evaluate phosphorylated biomarkers HS1, ZAP70, and PLCγ2 in CD8⁺ T cells.

Whole blood was collected in a collection tube coated with sodium heparin.

Compound dilution series (7-points, 0.01, 0.1, 1, 10, 50, 100, 1000 nM) were prepared in media and added to cells. Blood samples were pre-incubated with compound for two hours at 37° C. at concentrations ranging from 0.01-1000 nM with a final DMSO concentration of 0.05%.

After incubation with compound, 100 μL blood from each indicated concentration per donor was subsequently transferred to a 2.4 mL deep well Master Block plate for pharmacodynamic analysis. 2 μg/mL mouse anti-CD3 and mouse anti-CD28 were added to each well for 5 minutes.

After 5 minutes, 30 μg of goat anti-mouse IgG (H+L) antibody (Invitrogen) was added to cross-link the stimulation antibodies and induce synchronized cellular stimulation. Cells were stimulated alongside non-stimulated & stimulated DMSO controls for 60 minutes.

All buffers (Phosflow Lyse/Fix Buffer 5×, Phosflow Perm Buffer I; BD Biosciences) were prepared according to the manufacturer's instructions. After 60 minutes of antibody stimulation, one volume of blood was mixed with 20 volumes of pre-warmed BD Phosflow Lyse/Fix Buffer (1×) by repeated pipetting (8-10 times), then transferred to a 37° C. water bath for 10 min. Cells were washed and centrifuged at 500×g for 5 min (2×) with PBS. Following centrifugation, cell pellets were resuspended in cold BD Phosflow Perm Buffer I (BD) and incubated at 4° C. for 20 min.

Endogenous Fc receptors were blocked using human BD Fc block (2.5 μg/mL/100 total volume) prepared in 1×PBS for 10 min at 4° C., and cells were stained with Alexafluor700 mouse anti-NHP CD45 (BD Biosciences; 5 μg/mL), Brilliant Violet 605 mouse anti-human CD8 (BD Biosciences; 5 μs/ml), AlexaFluor-647 mouse anti-human phosphorylated Y759-PLCγ2 (BD; 5 μg/ml) and Phycoerythrin (PE) mouse anti-rabbit Y397-HS1 (Cell signaling Technology; 1:50), and AlexaFluor-488 mouse anti-human phosphorylated Y319/Syk (Y352)-ZAP70 (BD; 5 μg/ml) and incubated for 1 hr at 4° C. After 1 hr of incubation, cells were washed twice with Phosflow Perm Buffer I (BD Biosciences) and resuspended in 200 μL PBS for flow cytometric analysis.

Stained cells were run on an Attune NxT Acoustic Focusing Flow Cytometer (Thermo Fisher, cat. No. A29004), and data was analyzed using FlowJo (v10.6.1) and GraphPad Prism (v8.00) software. Single lymphocytes were gated for CD8⁺ cells (CD45⁺/CD8⁺) cells, and population frequencies were calculated for each population. The data is quantified by the percent positive. Using unstimulated pre-dose control for each animal as a baseline, a gate is placed at the 90th percentile for that specific cell population and phosphoprotein. As protein becomes phosphorylated the fluorescence increases resulting in a larger fraction of the cells entering that gate. Each population was evaluated for % positive pY759-PLCγ2, pY397-HS1 levels, pY319/Syk (Y352)-ZAP70 levels.

Using a flow cytometry assay, we assessed the kinetics of phosphorylated HS1 and PLCγ2, and ZAP70 in cynomolgus macaque CD8⁺ T cells. Compound 23 induced a dose-dependent increase in phosphorylated HS1 and PLCγ2 in the presence of anti-CD3 and anti-CD28 co-stimulation (FIG. 1-2). Average mean EC50±standard deviation calculated for phosphorylated PLCγ2 and phosphorylated HS1 and phosphorylated ZAP70 were 121±37.4 nM and 230±150.2 nM, and 173±177 nM, respectively.

These data confirmed specific proximal biomarker changes in Compound 23 treated whole blood from multiple cynomolgus macaques and demonstrated the pharmacodynamic effects of Compound 23 in vitro using a flow cytometry-based assay.

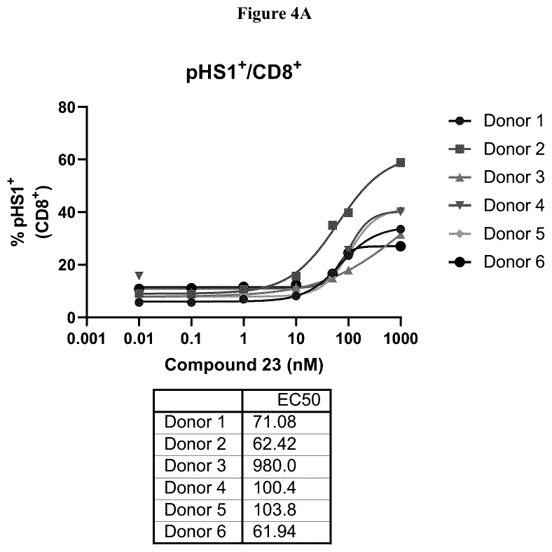

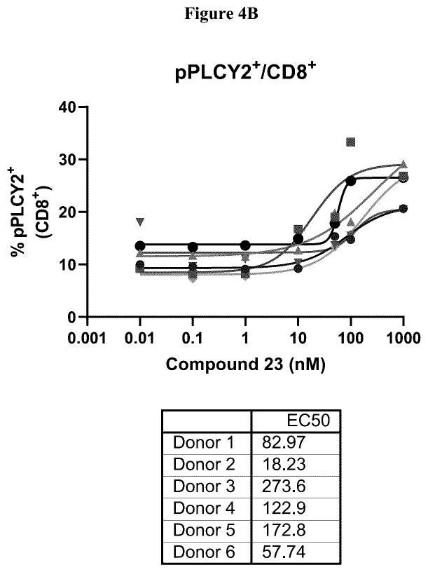

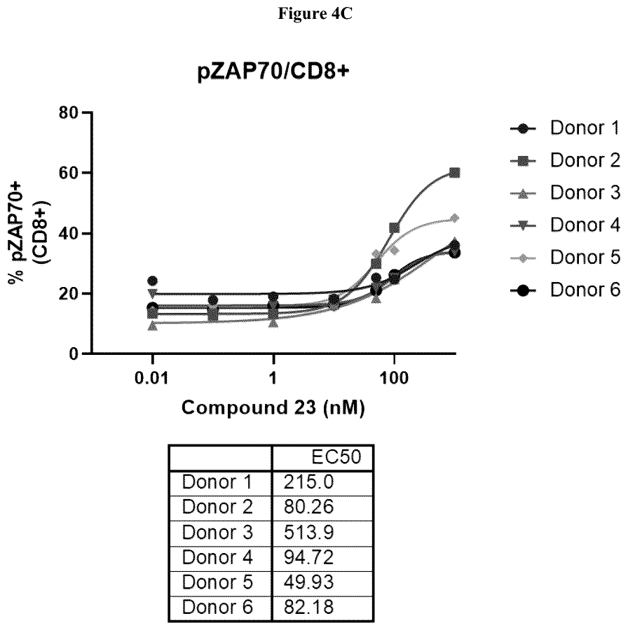

FIG. 4A provides Compound 23 inducing phosphorylated HS1 upon stimulation in vitro in cynomolgus macaque CD8⁺ T cells from whole blood. Whole blood from six cynomolgus macaque donors collected in sodium heparin anti-coagulant was treated with increasing concentrations of Compound 23 (0.01-1000 nM) for 2 hrs. at 37° C. Treated blood was stimulated with 2 μg/mL anti-CD3 & anti-CD28 for 5 mins and subsequently stimulated with crosslinker for 60 minutes. % phosphorylated HS1 was quantitated in T cells (CD8+) using flow cytometry.

FIG. 4B provides Compound 23 inducing phosphorylated PLCγ2 upon stimulation in vitro in cynomolgus macaque CD8⁺ T cells from whole blood. Whole blood from six cynomolgus macaques collected in sodium heparin anti-coagulant was treated with increasing concentrations of Compound 23 (0.01-1000 nM) for 2 hrs. at 37° C. Compound treated blood was stimulated with 2 ug/mL anti-CD3 & anti-CD28 for 5 mins and subsequently stimulated with crosslinker for 60 minutes. % phosphorylated PLCγ2 was quantitated in T cells (CD8+) using flow cytometry.

FIG. 4C provides Compound 23 inducing phosphorylated ZAP70 upon stimulation in vitro in cynomolgus macaque CD8⁺ T cells from whole blood. Whole blood from six cynomolgus macaques collected in sodium heparin anti-coagulant was treated with increasing concentrations of Compound 23 (0.01-1000 nM) for 2 hrs. at 37° C. Compound treated blood was stimulated with 2 ug/mL anti-CD3 & anti-CD28 for 5 mins and subsequently stimulated with crosslinker for 60 minutes. % phosphorylated ZAP70 was quantitated in T cells (CD8⁺) using flow cytometry.

### Example 4: Phosphorylated ITK, HS1, PLCγ2, PLCγ1, SYK, and ZAP70 Biomarker Assessment

To identify proximal biomarkers of CBL-B inhibition in immune cells, a panel of phosphorylated proteins were screened in human whole blood incubated with CBL-B inhibitor and concomitant anti-CD3 and anti-CD28 antibodies. The effect of CBL-B inhibition was assessed across multiple cellular signaling networks.

Readouts included apoptosis, βcatenin, cell cycle, cell stress, chaperones, cytokines, cytoskeletal rearrangement, DNA damage, endocytosis, epigenetics, insulin, interferon, MAPK signaling, metabolism, NF-κB signaling, oxidative stress, P38 signaling, phosphatase, PI-3 kinase, PKC, receptor tyrosine kinase, SAPK/JNK, second messenger, SMAD, T-cell signaling, transcription factor, and tyrosine kinase.

Reagents included mouse anti-human CD3 (Clone: UCHT1; BD Biosciences/550368), mouse anti-human CD28 (Clone: CD28.2, BD Biosciences/556620), goat anti-mouse IgG (H+L) (Invitrogen/A16068), Phospho Fix/Lyse (BD Biosciences/558049), Phospho Perm Buffer I (BD Biosciences/557885), and DMSO (Sigma, D2650-5x5ML). Phosphorylation and surface stain antibodies included pY180/pY511-ITK (Pe) (BD/Cat: 562753), pY397-HS1 (Pe) (CST/Cat: 11880), pY759-PLCg2 (Pe) (BD/Cat:558490), pY783-PLCg1 (AlexaFluor 647) (BD/Cat: 557883), pY525/pY526/pY348-SYK (Pe) (BD/Cat: 558529), pY319-Zap70 (Pe-Cy7) (BD/Cat:561458), CD3 (BUV805) (BD/Cat:565515), CD4 (BUV737) (BD/Cat:564306), CD3 (BV786) (BD/Cat:563823).

Human heparinized whole blood from 8 donors was received after an overnight shipment at 4° C. The blood was plated, treated with CBL inhibitor at 10 uM and incubated at 37° C. for 60 minutes. After incubation, the cells were stimulated with anti-CD3/CD28 followed by a cross-linking antibody (30 μg of goat anti-mouse IgG (H+L) antibody (Invitrogen)). After 60 minutes, the cells were fixed, and stained using surface markers for T cells (CD3⁺CD4⁺& CD3⁺CD8⁺) and analyzed by flow cytometry for target phosphorylation of the following markers: pY180/pY511-ITK, pY397-HS1, pY759-PLCg2, pY783-PLCg1, pY525/pY526/pY348-SYK & pY319-Zap70.

Fold change of each biomarker was calculated with the following example equation:

[% phosphorylated signal of CBL inhibitor treated sample (Inhibition of CBL) upon stimulation/% phosphorylated signal of DMSO treated sample upon stimulation]

CBL inhibition increased phosphorylation of PLCγ1, SYK and ZAP70 2-3 fold compared to DMSO control. CBL inhibition increased phosphorylation of ITK, HS1, and PLCγ2 3-4 fold compared to DMSO control.

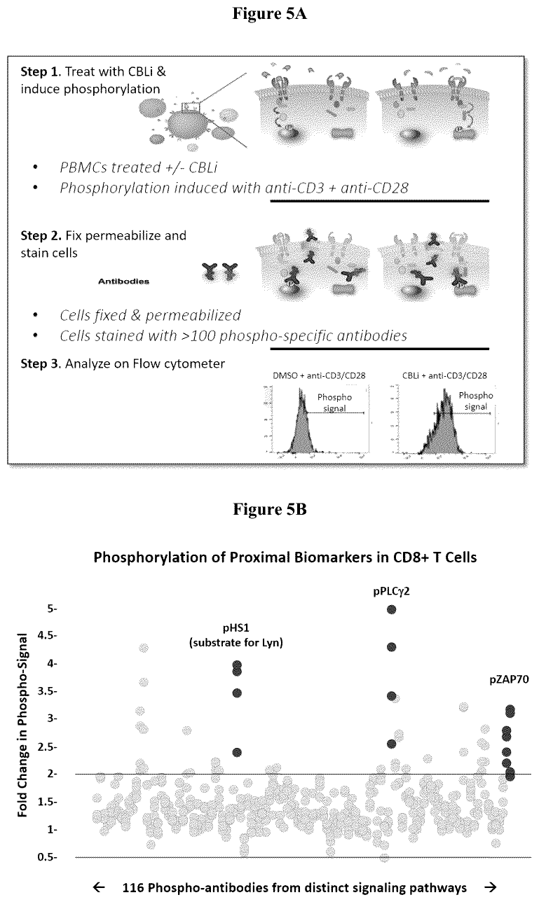

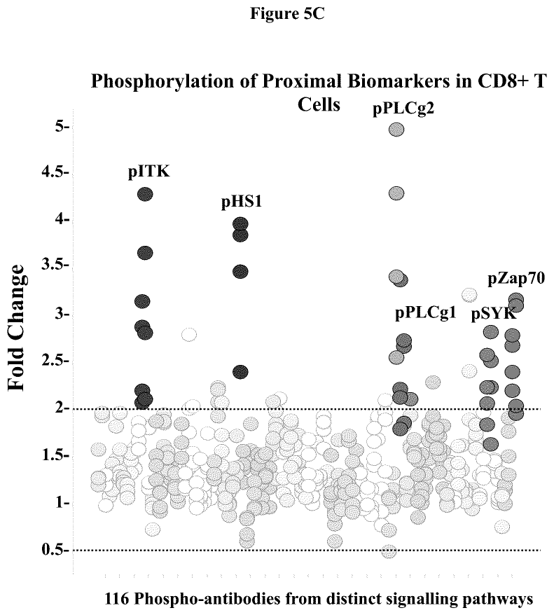

FIG. 5A provides an illustration of methods for identifying proximal biomarkers of Compound 23 described herein.

FIG. 5B provides phosphorylation of proximal biomarkers in CD8+ cells, identifying markers pHS1, PLCγ2, and pZAP70.

FIG. 5C provides phosphorylation of proximal biomarkers in CD8+ cells.

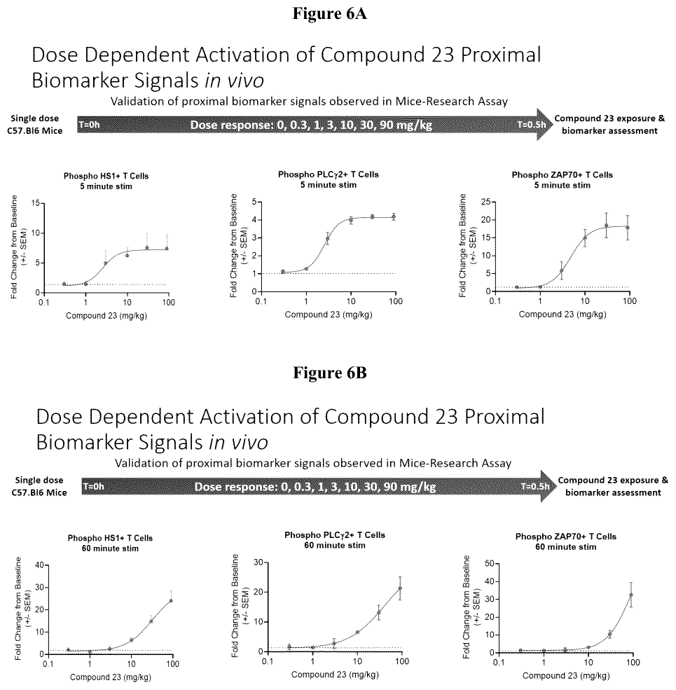

Whole blood from eight human donors was collected in sodium heparin anti-coagulant treated with 10 μM of Compound 23 for 1 hr at 37° C. CBL inhibitor treated blood was incubated with 2 μg/mL anti-CD3 & anti-CD28 for 5 minutes and subsequently stimulated with crosslinker for 60 minutes. Fold change of % phosphorylation normalized to DMSO was quantitated in T cells (CD8⁺) using flow cytometry is provided in FIGS. 6A-6B.

### Example 5: Compound 23: Human Pharmacodynamic Model Development

Compound 23 is a CBL-B inhibitor that has demonstrated remarkable potential as a novel therapy for cancer. CBL-B is a key negative regulator of immune function, and its upregulation has been found in various cancer types, contributing to immune evasion and tumor progression. Preclinical studies have shown that inhibiting CBL-B activity can enhance immune response and improve antitumor immunity, making it a promising target for cancer immunotherapy.

A clinical trial of Compound 23 aims to evaluate the efficacy of this CBL-B inhibitor by examining proximal biomarkers of CBL-B inhibition through a phosphorylation flow cytometry assay. This assay is based on the detection of changes in phosphorylation levels of key signaling proteins downstream of CBL-B, including HS1, PLCγ2, and ZAP70, in response to Compound 23 treatment. By utilizing this approach, researchers can quantitatively assess intracellular signaling events and identify potential predictive biomarkers of response to CBL-B inhibition, thereby providing a better understanding of the mechanism of action of Compound 23.

Compound 23 is being assessed in a first-in-human, multicenter, open-label, Phase 1 dose escalation and expansion trial designed to evaluate Compound 23 in patients with relapsed or refractory solid tumors from a variety of indications, including platinum-resistant epithelial ovarian cancer (EOC), gastric cancer, squamous cell carcinoma of the head and neck (HNSCC), metastatic melanoma, non-small cell lung cancer (NSCLC), metastatic castration-resistant prostate cancer (mCRPC), malignant pleural mesothelioma (MPM), triple-negative breast cancer (TNBC), locally advanced or metastatic urothelial cancer, cervical cancer, microsatellite stable colorectal cancer (MSS CRC), and diffuse large B-cell lymphoma with Richter transformation (DLBCL-RT). The primary objective is to establish the safety and tolerability of Compound 23, characterize pharmacokinetics and pharmacodynamics (PK/PD) including biomarkers, and determine the recommended Phase 1b dose. Patients with metastatic or unresectable disease who have progressed after prior therapy and for whom standard therapy with proven clinical benefit does not exist, is no longer effective, or is not appropriate are eligible to participate in the trial.

The use of proximal biomarkers of CBL-B inhibition, such as phosphorylated HS1, in the assessment of CBL-B inhibitors like Compound 23 is a significant advancement in the field of cancer immunotherapy. The development of a novel assay, such as the phosphorylation flow cytometry assay used to assess the phosphorylation levels of HS1 in response to Compound 23 treatment provides a reliable and high-throughput approach to assess the effectiveness of CBL-B inhibitors, potentially leading to more accurate predictions of treatment efficacy and improved patient outcomes.

Human Pharmacodynamic Model Development

In order to identify proximal biomarkers of CBL-B inhibition in immune cells, a panel of phosphorylated proteins were screened in human PBMCs treated with CBL-B inhibitor and the CD3 T cell receptor and CD28 co-receptor antibody stimulation. The effect of CBL-B inhibition was assessed across the T cell receptor signaling network, and multiple proximal biomarkers of CBL-B inhibition were identified. Phosphorylated hematopoietic lineage cell-specific protein (HS1) was selected based on its magnitude of signal and reproducibility across multiple donors. HS1 binds the actin cytoskeleton and regulates TCR activation through the immune synapse (Yamanashi et al., 1993, Proc. Natl. Acad. Sci. USA 90(8):3631-3635; Scaife et al., 2003, J. Cell. Sci. 116(3):463-73, Gomez et al., 2006, Immunity 24(6):741-752; Carrizosa et al., 2009, J. Immunol. 183(11):7352-7361). CBL-B is known to limit signaling following TCR engagement. Increased phosphorylation of HS1 in TCR stimulated T cells after CBL-B inhibition by Compound 23 identifies a biologically relevant biomarker for monitoring pharmacologic inhibition of CBL-B in human PBMCs.

Based on the above studies, human primary whole blood in vitro stimulation assays were considered the most biologically relevant assays and included monitoring of the phosphorylation of HS1 by flow cytometry. To evaluate target engagement of CBL-B inhibition, proximal biomarker phosphorylated HS1 was identified and characterized in vitro. Fresh whole blood obtained from multiple human donors (n=6) (BioIVT) were collected in a collection tube coated with sodium heparin. Compound dilution series (10-fold dilutions in DMSO) were prepared in media and pre-incubated with whole blood cells for two hours at 37° C. The Compound 23 concentration range selected was based on comparisons of compound concentrations from several in vitro and in vivo studies associated with biologic activity. After compound incubation, samples were added directly into a 96 deep well master block plate for acute assessment of phosphorylated biomarkers. For assessment of phosphorylated biomarkers in humans, the CD3 T cell receptor and the CD28 co-receptor on T cells were stimulated with anti-CD3 and anti-CD28 co-stimulation and were subsequently crosslinked to induce cellular stimulation.

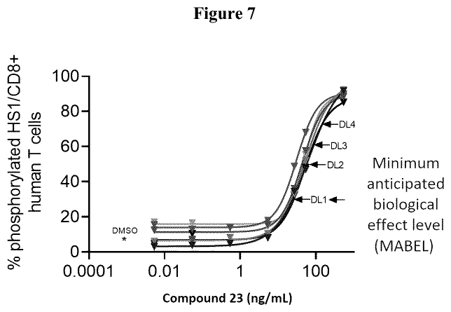

Compound 23 elicited a concentration-dependent increase in phosphorylated HS1. Maximum phosphorylation of HS1 and minimal variability between donors is observed upon CBL-B inhibition, suggesting that HS1 is directly phosphorylated and is thus a specific and robust marker of CBL-B inhibition, for this reason, it was prioritized for minimum anticipated biological effect level (MABEL) dose calculations (FIG. 7).

HS1 Phosphorylation and Comparison to Anti-Tumor Efficacy in Mice

To assess the in vivo effects of Compound 23 on candidate clinical proximal biomarkers of CBL-B inhibition and compare them to anti-tumor efficacy, two experiments were performed in mice. In the first experiment, whole blood was obtained from groups of mice (n=4 per group) administered a single oral dose of either vehicle (0.5% MC/0.2% polysorbate 80 in deionized water) or increasing doses of Compound 23 at 0.3, 1, 3, 10, 30, or 90 mg/kg. The response to Compound 23 on HS1 activation was measured in anti-CD3 and anti-CD28 stimulated CD8⁺ T cells using flow cytometry. Compound 23 induced a dose-dependent increase in the proportion of phosphorylated HS1⁺CD8⁺ in the presence of anti-CD3 and anti-CD28 co-stimulation. The greatest induction of phosphorylated HS1 was observed upon ex vivo stimulation following administration of Compound 23 doses of ≥10 mg/kg. The minimum biological effect level observed in mice (EC50) was 12 mg/kg. Statistical significance was calculated using 2-way ANOVA with Dunnett's multiple comparisons test.

The preclinical murine in vivo data using the A20 lymphoma tumor model demonstrates the dose-dependent anti-tumor activity of Compound 23, resulting in significantly smaller tumors and prolonged median survival time when dosed at 10, 30 or 60 mg/kg compared to the vehicle control. An elevated percentage of pHS1+CD8+ T cells correlated with potent anti-tumor effects in mouse syngeneic tumor models and defined a biomarker therapeutic range. At this therapeutic range, approximately 50% proximal biomarker pHS1 correlates with the efficacious range.

Assessment of Proximal Biomarker of CBL-B Inhibition, Phosphorylated HS1 in Compound 23-101

Pharmacodynamic analysis of the proximal biomarker, phosphorylated HS1 is assessed in circulating CD8⁺ T cells from patients that are being administered doses ranging from 5 to 50 mg of Compound 23 once daily and is being used to assess target engagement of Compound 23 as part of the dose/exposure-response profile. Whole blood is collected longitudinally and added into two types of TruCulture (Myriad) tubes. One tube contains media, and the second tube contains anti-CD3 and anti-CD28, which are subsequently crosslinked to induce cellular stimulation for pharmacodynamic analysis. Phosphorylated HS1 is assessed by flow cytometry pre and post dose on Cycle 1 Day 1 (C₁D1) and Cycle 2 Day 1 (C₂D1) to characterize single/multiple dose PD profiles.

% Maximal phosphorylated signal and % AUC are two commonly used parameters to assess the pharmacodynamic effects of drugs.

% Maximal phosphorylated signal refers to the maximum amount of a specific protein that becomes phosphorylated (i.e., modified by the addition of a phosphate group) in response to a drug or other stimulus. This parameter is often used to measure the activation of signaling pathways that are important for various cellular processes, including cell growth and differentiation, immune response, and metabolism. A higher % maximal phosphorylated signal indicates greater pathway activation, which may correlate with a more potent pharmacodynamic effect.

% AUC, on the other hand, refers to the area under the curve (AUC) over time, expressed as a percentage of the maximum possible AUC. In summary, both % maximal phosphorylated signal and % AUC are useful parameters to assess the pharmacodynamic effects of drugs. % maximal phosphorylated signal is a measure of the activation of signaling pathways, while % AUC reflects the duration of the effect on PD signals. Both parameters can be used to evaluate the potency and duration of a drug's pharmacodynamic effects.

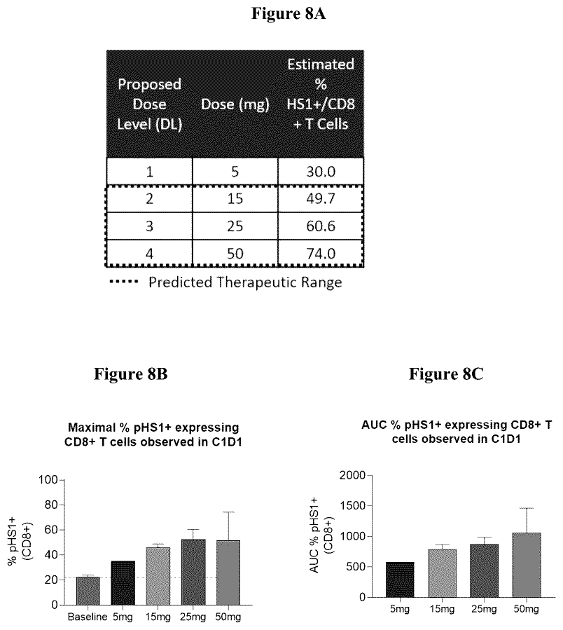

Interim PD results (FIG. 8A) demonstrate dose dependent increases of % maximal phosphorylated HS1 and % pHS1 AUC, which are consistent with human dose projection models. PD results (FIG. 8B) demonstrate % of phosphorylated HS1 expressing CD8+ T cells of 35% (5 mg, n=1), 46% (15 mg, n=12), 53% (25 mg n=7), and 52% (50 mg n=2) in cycle 1, day 1, and expression levels that correlated with anti-tumor efficacy in animal models. Further, dose dependent increases of % phosphorylated HS1 AUC over time were observed (FIG. 8C). PD results demonstrate increased phosphorylated HS1 AUC of 578 (5 mg, n=1), 787 (15 mg, n=12), 876 (25 mg n=7), and 1055 (50 mg n=2) in cycle 1, day 1. Correlation analysis was performed between PK parameters (AUC and CMAX) and pHS1 in CD8⁺ T cells at each dose level using Pearson's correlation coefficient. The results are presented as scatter plots with a line of best fit, where each point represents the mean value of PK parameter and pHS1 for an individual patient at a given dose level. The correlation coefficient (r) and its corresponding p-value are reported for each analysis. A positive trend of AUC % pHS1 with PK with statistically significant slope is observed (p=0.02).

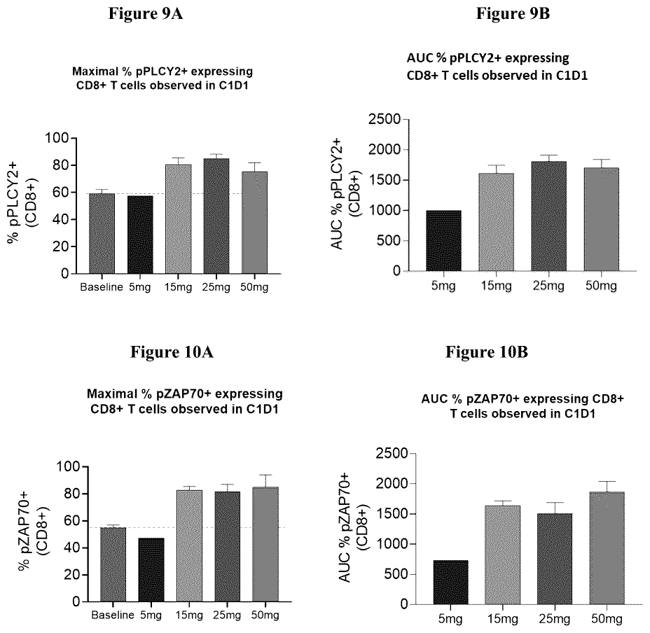

PD results (FIG. 9A) demonstrate % of phosphorylated PLCγ2 expressing CD8⁺ T cells of 56% (5 mg, n=1), 81% (15 mg, n=12), 85% (25 mg n=7), and 75% (50 mg n=2) in cycle 1, day 1, and expression levels that correlated with anti-tumor efficacy in animal models. Further, increases of % phosphorylated PLCγ2 AUC over time were observed (FIG. 9B). PD results demonstrate increased phosphorylated PLCγ2 AUC of 1003 (5 mg, n=1), 1616 (15 mg, n=12), 1807 (25 mg n=7), and 1706 (50 mg n=2) in cycle 1, day 1. No apparent trend of maximal or AUC % pPLCγ2 with PK was observed.

PD results (FIG. 10A) demonstrate % of phosphorylated ZAP70 expressing CD8⁺ T cells of 47% (5 mg, n=1), 83% (15 mg, n=12), 82% (25 mg n=7), and 85% (50 mg n=2) in cycle 1, day 1, and expression levels that correlated with anti-tumor efficacy in animal models. Further, increases of % phosphorylated ZAP70 AUC over time were observed (FIG. 10B). PD results demonstrate increased phosphorylated ZAP70 AUC of 734 (5 mg, n=1), 1641 (15 mg, n=12), 1506 (25 mg n=7), and 1870 (50 mg n=2) in cycle 1, day 1. Positive trend of maximal % pZAP70 with PK (but not statistically significant; p=0.06).

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

1. A method for detecting the amount of CBL in a sample, comprising the steps of:
a. measuring the amount of a CBL biomarker in the sample; and
b. identifying the amount of CBL in the sample based on the amount of the CBL biomarker detected in the sample.

2. A method for detecting the amount of CBL in a cell, tissue, or organism, comprising the steps of:
a. measuring the amount of a CBL biomarker in a first sample of the cell, tissue, or organism;
b. measuring the amount of a CBL biomarker in a second sample of the cell, tissue, or organism; and
c. identifying a change in the amount of CBL in the cell, tissue, or organism based on the change in the amount of the CBL biomarker detected in the sample.

3. A method of treating a patient in need thereof with a CBL inhibitor comprising the steps of:
a. administering to a patient a first dose of a CBL inhibitor; and
b. measuring the amount of a CBL biomarker in a first sample of the patient;
c. identifying the amount of CBL in the patient based on the amount of the CBL biomarker detected.

4. A method of treating a patient in need thereof with a CBL inhibitor comprising the steps of:
a. administering to a patient a first dose of a CBL inhibitor;
b. measuring the amount of a CBL biomarker in a first sample of the cell, tissue, or organism;
c. measuring the amount of a CBL biomarker in a second sample of the cell, tissue, or organism; and
d. identifying a change in the amount of CBL in the cell, tissue, or organism based on the change in the amount of the CBL biomarker detected in the sample.

5. The method of claim 2, wherein an increase in the amount of the CBL biomarker indicates a decrease in the amount of CBL.

6. The method of claim 2, wherein a decrease in the amount of the CBL biomarker indicates a decrease in the amount of CBL.

7. The method of claim 4, wherein the first sample is collected prior to the administering, and the second sample is administered after the administering.

8. The method of claim 3, wherein the measuring of the first sample precedes the administering of the first dose.

9. The method of claim 3, wherein the first dose is selected based on the amount of CBL identified.

10. The method of claim 3, further comprising administering a second dose of the CBL inhibitor, wherein the dose is selected based on the amount of CBL identified.

11. The method of claim 10, wherein the second dose is increased if the amount of the CBL biomarker in a sample is below a predetermined range; wherein the second dose is maintained if the amount of the CBL biomarker in a sample is within a predetermined range; and/or wherein the second dose is decreased if the amount of the CBL biomarker in a sample is above a predetermined range.

12. The method of claim 11, wherein the second dose is decreased if the amount of the CBL biomarker in a sample is below a predetermined range; wherein the second dose is maintained if the amount of the CBL biomarker in a sample is within a predetermined range; and/or wherein the second dose is increased if the amount of the CBL biomarker in a sample is above a predetermined range.

13. The method of claim 11, wherein the second dose is increased, decreased, or maintained relative to the first dose.

14. The method of claim 1, wherein activity of CBL in the sample is identified.

15. The method of claim 1, wherein expression of CBL in the sample is identified.

16. The method of claim 1, wherein the amount of CBL protein in the sample is identified.

17. The method of claim 1, wherein inhibition of CBL activity in the sample is identified.

18. The method of claim 1, wherein inhibition of CBL expression in the sample is identified.

19. The method of claim 1, wherein degradation of CBL protein in the sample is identified.

20. The method of claim 1, that is conducted in vitro.

21. The method of claim 1, that is conducted ex vivo.

22. The method of claim 1, wherein the sample is from a cell culture.

23. The method of claim 1, wherein the sample is from a patient.

24. The method of claim 1, wherein the sample is a patient blood sample.

25. The method of claim 1, wherein the sample is a patient biopsy.

26. The method of claim 1, wherein the CBL biomarker is detected by immunoassay, western blotting, ELISA, mass spectrometry, immunohistochemistry or flow cytometry.

27. The method of claim 1, wherein the CBL biomarker is detected by flow cytometry.

28. The method of claim 1, wherein the disease or condition is a cancer.

29. The method of claim 1, wherein the disease or condition is a solid tumor.

30. The method of claim 1, wherein the disease or condition is a hematological cancer.

31. The method of claim 1, wherein the CBL biomarker is selected from phosphorylated interleukin-2-inducible kinase (pITK), phosphorylated hematopoietic lineage cell-specific protein (pHS1), phosphorylated phospholipase C2 (pPLCγ2), phosphorylated phospholipase C1 (pPLCγ1), phosphorylated spleen tyrosine kinase (pSYK), and phosphorylated Zeta-chain-associated protein kinase 70 (pZAP70).

32. The method of claim 1, wherein the CBL biomarker is phosphorylated interleukin-2-inducible kinase (pITK).

33. The method of claim 1, wherein the CBL biomarker is phosphorylated hematopoietic lineage cell-specific protein (pHS1).

34. The method of claim 1, wherein the CBL biomarker is phosphorylated phospholipase C2 (pPLCγ2).

35. The method of claim 1, wherein the CBL biomarker is phosphorylated phospholipase C1 (pPLCγ1).

36. The method of claim 1, wherein the CBL biomarker is phosphorylated spleen tyrosine kinase (pSYK).

37. The method of claim 1, wherein the CBL biomarker is phosphorylated Zeta-chain-associated protein kinase 70 (pZAP70).

38. (canceled)

39. (canceled)

40. (canceled)

41. (canceled)

42. (canceled)

43. (canceled)

44. A kit comprising a pharmaceutical composition of a CBL inhibitor and means for detecting a CBL biomarker.

45. The method of claim 1, wherein the CBL is selected from c-Cbl and Cbl-b.

46. The method of claim 1, wherein the CBL is Cbl-b.

47. The method of claim 1, wherein the CBL is c-Cbl.

48. The method of claim 1, wherein the CBL is Cbl-b and c-Cbl.
